SEQUENCE LISTING (4 sequences)

SEQ ID NO: 1 - DNA, 377 bp, Homo sapiens
ggatcctctcttgtagactttattcttcaggaccacaggaactcaccagggttacagaccctatttggaggctctgctagcacatttctagtgtaactaccagggaacgtgagctatactagtctgtgcccttgtttactagactgatcccacagaaatcccagtaggagtcacaagatgctgggatcggatcctctcttgtagactttattcttcaggaccacaggaactcaccagggttacagaccctatttggaggctctgctagcacatttctagtgtaactaccagggaacgtgagctatactagtctgtgcccttgtttactagactgatcccacagaaatcccagtaggagtcacaagatgctgggatcc

SEQ ID NO: 2 - DNA, 297 bp, Homo sapiens
atcccgaaaagcagacctgcttctccctgtccagccggttccccttccccttgcagtcggccccctgcatccgcgtcctccctgccagtcgagggtccccagctccaactccaccctcccagctgtgcgttcatagcgaccgccctccctgtagggacgcacggatctggtggtggagtcttggccggcaggactggacaggaaccgaaggggcgaggcgggtccgggggtggtgcgctccaattgggtgctgtccccagggggtggggcctgatcccctatttcccggcgcgccgg

SEQ ID NO: 3 - DNA, 508 bp, Homo sapiens
cggatccctcctcaaggctgagcacttggcatcgtccagtgaatctcaaccccggaagtcacagccttagagagcatggaaatggcccgaaggggactcagttccatgtcagaggctgggaactgcctttaggatgtgctatgttttgaattcatacccaaaatttacctcatttcctgtgatagccagaatttatggagctggacagtaagaggttaagatgagggtgatagctctatggttacaaacaataagtcactttcaaaaatcttgtttacaatacccctaactcagagacttttaagtctagaagtttcagtatctaaggtgggaatgcctcctgcaaggctgacaaaagtggtttcagaggtgcccatagctggcttctcatgctccctgatgtagggatagcaatgacgtcactgggttagatataatcatggatcatgatcatccagggaagctcatgttgttactacacagcaggacatgaggaatcgagggatcc

SEQ ID NO: 4 - DNA, 3747 bp, Homo sapiens
ggatcctctccacatcctccctgagtgctcccagaatcttcctgcagtcctccccatgttctctcaggcgtgggctgctgtacctggattggccctagcaggtgactcgggctggagtttggttgacaccagtggagccacaagcctgctgtgcacaggtgtgatggcaggcctgtgagggttcggggctgcatggccttgctccctttgcactggtgtctggatgtgctcagaggccccctgggtccccaggcctctgggacaaggccggttgagtctcaaaaacaggtaggaccccaagcagagccaaggcatcaccagccccagcccttgttcccgtgtgccccatctcccgaagcactcccctgtgtcatgcggtaccagctctgcctctgactccccatgcagtggccctaaggccaccccttgtcagtgtcctcctgggcctcctggggcgggcaagaacctgctcacacaggtacatgcacagcaagcatcggagggtctccttccctgggagacatcaccgccccacagtgggtcaccctcaaggctaaccactcagcttccgggtggcaagcctgcagagtggccccaggcatgccgggccaccttctaagtgtgccagtcccaccctgtgtgtgtgtgtggatgtgccagagatgtgcagtcacacttccatctgtgtcccatgcccccatgagtgtgtcctcacacctcttgccccgcctccctgctgtgtgcccctcgtagcagcttggccctgcccgctgcaccatgtgtacacagaggcttattttctctgcctttcgggccaggggcgtgctcgtaaattgtgcagtcgacgacacatttatcccgcgggcggctggcggtgtgaatttatggctgcacccccttcctggctgaggcaggacaggggccgggatacctctcaggcaggaacctccagtccactccactgggctgttgtggggccatgggggcgggctgggccgggagggcttaggagtgctcccaaggtctggtcttgcacagaaaccctgcaacttcagggtgcttggggcaatgagaggcgaggccactctgcctgggtgagggctgggggcacagtggctgcccacgtgccagaggcatggaggcccagagcccagcctaatgtcccagcctccatgcccccttccagtcggcttctcatcttcccacctaagaaagaggggacaacttggatgccccagggacagcaggtgtgggggtggctcccctcaggtgctgagtcagccacccactgccactaggcccactgggagtactgagatctggggaggactgtcagaaagggtgggggcctcgagggcagctggggctgcacacagggaagaggagcggagatactgtcgaatcagagcagagagcccctcagagctcacctgccctcctctgccttgtaaagatggggaaactgaggcccagggagagcagaggtcccaggtcacatgggtaggataacagagcctcaagatcctggattcctgctccctagtatgcaataaagggggtcctaggcacacccctccccacggagccacctttgtcctgcaaagtctggaggtggggcctccggctgaagcctcaacagggagctgatggagcaaattggcaagctggtcccatcagggccacagctggggcctgggacctggctgcctcccctccgtccgtcggtctgtctgtgtgcagggtcgggtctccaggggaccctctgtcggctctcaagctccctcccatctcggccccagccccccctcaccgcccgctgtcaggatgagcgatggccgtgcccctccccggccctcggccccaggcccggttcccgctcattagccactgacactgtttgctttcccgccatggacgcccaccccgtcacaggccattt[450 bp omitted]cccctcaggcagccccagtacacagcctggggggtgggtcctgaggcagcaggcacccctccctcctgcaccacccccgcccccaatatggccacagctgggcggcatcagggcccgcaggccagtggcagccagtccttcacttaaaaatgtgtttgtgatttcggcagcgaggcagataacggtgacgaatggcccgcctgccccccagggccctcagcccatctggtttgactttggcctgtcggcagtgcccttgggcctcactcctgccctctggcagtgccctccgagtgaggcacccctcaacctttgcacactctggttcctttgctggaaatgcccctcctctccacccatctgtccccccatcatgggctcccctggctatgcccataggagccacccatgcaacactcggggtggcaggtcctgggcagtgctggggtttgcagggtgggggagggcacaggcccagcagggagggggcatgccttgcctgggccctgcatcccctctgtctaggagggaatagtgagtgcccacctaaggccagaggggccgaggctaaggtgacggagtgggccaggaccgagagaggtggcccggcggccagggcagggcctagccctgtgagacagcccatgtggtcattgtcaggaggaatttctgacagggctcgggaccctcctgaccatcgattatccaagcaggaggagtggcttccgggtccggctgtgggctcagctgggaggtcactgaggtcaagcccgagtgcccctcctcctccagtctccccttctactcccttggggctccccagttgggcagaggtgcctggaaggttgctcctgcccagtttgaatcctggccagctaccacagggagtggtcaggggtccactagggactggagctcatgcattccttagcacaacttcccaatcaacccctgcgtgcccaggtcacagacggagtcagacctggccctcagagaggagataaggagaggtggaggtgcttagagctcccctggcccatctcaggggttggggaggggcttgtgtagagggagggcattcgggcaggatagaggaggcagccctccctgggggtctggggtcgcctccaggagaggcactgctccattccccactgcaccctccaggcccactccctgccctgtggcgaggacagctgtagggggaggggaggggcagcgccgtgtcaggacccccaccccctcctccaaagcaggaaaatccctctccgttagtatcacattgttgagaattaactttgttgaaataaaaattggtcgtggtattaactcggcctcaaggattttctctgggatcc

(19) United States
(12) Patent Application Publication
(10) Pub. No.: US 2003/0037351 A1
(43) Pub. Date: Feb. 20, 2003

(54) Nucleic acid regulatory sequences and uses therefor

(76) Inventors: Donald C. Lo, Chapel Hill, NC (US); James B. Antczak, Durham, NC (US); Shawn Barney, Henderson, NC (US); Howard M. Bomze, Durham, NC (US)

Correspondence Address: PENNIE AND EDMONDS, 1155 AVENUE OF THE AMERICAS, NEW YORK, NY, 100362711

(21) Appl. No.: 10/165,828

(22) Filed: Jun. 6, 2002

Related U.S. Application Data
(60) Provisional application No. 60/296,192, filed on Jun. 6, 2001.
(60) Provisional application No. 60/296,194, filed on Jun. 6, 2001.
(60) Provisional application No. 60/296,304, filed on Jun. 6, 2001.
(60) Provisional application No. 60/296,305, filed on Jun. 6, 2001.

(51) Int. Cl. A01K 67/00; C12Q 1/68; C07H 21/04; C12N 9/00; C12P 21/02; C12N 5/06

(52) U.S. Cl. 800/8; 435/69.1; 435/183; 435/320.1; 435/325; 536/23.2; 435/6

(57) ABSTRACT

The present invention is directed to the nucleotide sequences of the CNI-01054, CNI-01056, CNI-01058, or CNI-01059 regulatory sequences, and to transcription activating regulatory molecules derived therefrom. The invention is further directed to vectors comprising these sequences, and to host cells containing the vectors. The invention further provides methods for the expression of a nucleotide sequence, or producing a polypeptide, of interest using the CNI-01054, CNI-01056, CNI-01058, or CNI-01059 regulatory sequences, in vitro and in vivo. Also provided is a method of identifying a regulator of the CNI-01054, CNI-01056, CNI-01058, or CNI-01059 regulatory sequences. Kits and non-human transgenic animals containing the CNI-01054, CNI-01056, CNI-01058, or CNI-01059 regulatory sequences are also provided.

This application claims benefit of U.S. Provisional Application No. 60/296,192, filed Jun. 6, 2001; U.S. Provisional Application No. 60/296,194, filed Jun. 6, 2001; U.S. Provisional Application No. 60/296,304, filed Jun. 6, 2001; and U.S. Provisional Application No. 60/296,305, filed Jun. 6, 2001.

### 1. INTRODUCTION

The present invention relates to nucleic acid regulatory sequences that modulate (e.g., promote, enhance, suppress, repress, or silence) expression of a nucleic acid of interest in a cell. In particular, the present invention relates to nucleic acid regulatory sequences referred to herein as the CNI-01054 regulatory sequence, the CNI-01056 regulatory sequence, the CNI-01058 regulatory sequence, the CNI-01059 regulatory sequence, and transcription-modulating sequences thereof. In a specific embodiment, the present invention relates to the CNI-01054 regulatory sequence, or the CNI-01056 regulatory sequence, or the CNI-01058 regulatory sequence, or the CNI-01059 regulatory sequence, or portions thereof, that promote or enhance transcription of nucleic acids of interest in cells, in particular cells of the nervous system, including, but not limited to cells in the central nervous system (CNS), such as neurons and glia in the brain. The present invention also relates to vectors and cells engineered to contain such regulatory sequences. The present invention still further relates to methods of using the regulatory sequences of the invention to modulate expression of a nucleic acid of interest in cells, preferably cells of the nervous system.

### 2. BACKGROUND OF THE INVENTION

The molecular basis of nervous system-specific gene expression is relatively poorly understood, mainly because of the large number of diverse cell types in the mammalian nervous system. Although many of the genes expressed in the nervous system are “housekeeping” genes, expressed in a variety of tissues, a significant number of genes have been identified that are expressed exclusively in neurons or glia. What is known regarding the molecular basis of gene expression in the nervous system has been reviewed elsewhere (Twyman & Jones, J. Neurogenet. 10(2):67-101 (1995); Quinn, Prog. Neurobiol. 50(4):373-79 (1995); Grant, in MOLECULAR BIOLOGY OF THE NEURON Davies & Morris, eds., Bios Scientific Publishers, Oxford (1996)).

Promoters of nervous system-specific genes have been used to direct the expression of heterologous genes to nervous system-derived cells in culture or in transgenic animals. For example, the use of nervous system-specific promoters to express heterologous genes in cell culture or in transgenic mice has allowed the creation of disease models (Sturchler-Pierrat & Sommer, Rev. Neurosci. 10(1):15-24 (1999); Brenner, Brain Pathol 4(3):245-57 (1994)), permitted the characterization of individual gene function (Caroni, J. Neurosci. Meth. 71(1):3-9 (1997)), and defined the minimum promoter and enhancer sequences necessary for tissue-specific expression (Chin et al., J. Biol. Chem. 269(28):18507-18513 (1994); Liu et al. Brain Res. Mol Brain Res. 50(1-2):33-42 (1997); Whyte et al., Mol. Endocrinol. 9(4):467-477 (1995); Min et al., Brain Res. Mol. Brain Res. 27(2):281-9 (1994)). Nervous system-specific promoters have also been used to deliver therapeutic genes to the CNS to correct genetic deficiencies in vitro and in vivo (Kaplitt et al., Nature Genet. 8(2):148-54 (1994); Miyao et al., Jpn. J. Cancer Res. 88(7):678-86 (1997); Hayward, Chem. Senses 20(2):261-9 (1995)). In addition, in vitro binding assays, mutational analysis and sequence analysis have been used to identify and map the cis-acting regulatory regions and trans-acting factors that impart tissue-specificity and regulatory characteristics to the promoter.

Although the identification and characterization of promoters and enhancers functional in nervous system cells has given us fundamental insights into the regulation of gene expression in the nervous system, the picture is far from complete. Thus, there continues to be a need for the discovery of additional regulatory sequences that are functional in nervous system cells and especially a need for information serving to specifically identify and characterize them in terms of their DNA sequence.

### 3. SUMMARY OF THE INVENTION

The present invention relates to nucleic acid regulatory sequences that modulate (e.g., promote, enhance, suppress, repress, or silence) expression of a nucleic acid of interest in a cell. In particular, the invention relates to an isolated nucleic acid molecule comprising the nucleotide sequence of SEQ ID NO: 1, SEQ ID NO: 2, SEQ ID NO: 3, or SEQ ID NO: 4. In one embodiment, then, the invention relates to an isolated nucleic acid regulatory sequence molecule comprising a transcription activating nucleotide sequence of SEQ ID NO: 1, SEQ ID NO: 2, SEQ ID NO: 3, or SEQ ID NO: 4. In specific embodiments, the invention relates to an isolated nucleic acid regulatory sequence, wherein the isolated nucleic acid regulatory sequence molecule is a restriction fragment of SEQ ID NO: 1, SEQ ID NO: 2, SEQ ID NO: 3, or SEQ ID NO: 4. In another specific embodiment, the invention relates to an isolated nucleic acid regulatory sequence, wherein the isolated nucleic acid regulatory sequence is created by nuclease digestion of a nucleic acid molecule comprising SEQ ID NO: 1, SEQ ID NO: 2, SEQ ID NO: 3, or SEQ ID NO: 4. In another specific embodiment, the invention relates to an isolated nucleic acid molecule of claim 1, wherein the isolated nucleic acid molecule is operably linked to a nucleic acid molecule comprising a coding sequence. In another specific embodiment, the invention relates to an isolated nucleic acid regulatory sequence molecule of any one of the preceding, wherein the isolated nucleic acid regulatory sequence molecule is operably linked to a nucleic acid molecule comprising a coding sequence. In another embodiment, the invention relates to an isolated nucleic acid molecule comprising the reverse complement of the nucleotide sequence of SEQ ID NO: 1, SEQ ID NO: 2, SEQ ID NO: 3, or SEQ ID NO: 4. In another specific embodiment, the invention relates to an isolated nucleic acid regulatory sequence molecule comprising the reverse complement of the nucleotide sequence of the nucleic acid regulatory sequence. In another specific embodiment, the invention relates to an isolated nucleic acid regulatory sequence molecule, wherein the transcription activating nucleotide sequence comprises at least about 50 contiguous nucleotides of SEQ ID NO: 1, SEQ ID NO: 2, SEQ ID NO: 3, or SEQ ID NO: 4. In another specific embodiment, the invention relates to an isolated nucleic acid regulatory sequence molecule, wherein the transcription activating nucleotide sequence comprises at least about 100 contiguous nucleotides of SEQ ID NO: 1, SEQ ID NO: 2, SEQ ID NO: 3, or SEQ ID NO: 4. In another specific embodiment, the invention relates to an isolated nucleic acid regulatory sequence molecule, wherein the transcription activating nucleotide sequence comprises at least about 200 contiguous nucleotides of SEQ ID NO: 1, SEQ ID NO: 2, SEQ ID NO: 3, or SEQ ID NO: 4.

The invention also provides nucleic acid sequences that hybridize to SEQ ID NO: 1, SEQ ID NO: 2, SEQ ID NO: 3 or SEQ ID NO: 4. Thus, in one embodiment, the invention relates to an isolated nucleic acid regulatory sequence molecule comprising a transcription activating nucleotide sequence that hybridizes over its entire length to the nucleotide sequence of SEQ ID NO: 1, SEQ ID NO: 2, SEQ ID NO: 3, or SEQ ID NO: 4 or the complement thereof.

The invention also provides for vectors containing a regulatory sequence of the invention. Thus, in one embodiment, the invention relates to a vector comprising the nucleotide sequence of SEQ ID NO: 1, SEQ ID NO: 2, SEQ ID NO: 3, or SEQ ID NO: 4. In a specific embodiment, the invention relates to a vector comprising at least 20, 50, 100, or 200 nucleotides of the nucleotide sequence of the nucleic acid regulatory sequence. In another specific embodiment, the invention relates to a vector comprising the reverse complement of SEQ ID NO: 1, SEQ ID NO: 2, SEQ ID NO: 3 or SEQ ID NO: 4, or a transcription activating nucleotide sequence thereof. In another specific embodiment, the invention relates to a vector containing an isolated nucleic acid regulatory sequence that hybridizes along its entire length to the sequence of SEQ ID NO: 1, SEQ ID NO: 2, SEQ ID NO: 3 or SEQ ID NO: 4. In another specific embodiment, the invention relates to a vector further comprising a coding sequence operably linked to the nucleotide sequence of SEQ ID NO: 1, SEQ ID NO: 2, SEQ ID NO: 3, or SEQ ID NO: 4, or a subsequence thereof. In a more specific embodiment, the invention relates to a vector comprising a coding sequence operably linked to the nucleotide sequence of SEQ ID NO: 1, SEQ ID NO: 2, SEQ ID NO: 3, or SEQ ID NO: 4, or a subsequence thereof, wherein the coding sequence is heterologous to the nucleotide sequence of SEQ ID NO: 1, SEQ ID NO: 2, SEQ ID NO: 3, or SEQ ID NO: 4. In another specific embodiment, any of the vectors described above further comprises a multiple cloning site (MCS), wherein when a nucleotide sequence is inserted into the MCS, the nucleotide sequence is operably linked to SEQ ID NO: 1, SEQ ID NO: 2, SEQ ID NO: 3, or SEQ ID NO: 4 or a subsequence thereof. In another more specific embodiment, the invention relates to a vector further comprising an internal ribosomal entry site (IRES). In another specific embodiment, the invention relates to a vector further comprising a multiple cloning site (MCS), wherein when a nucleotide sequence is inserted into the MCS, the nucleotide sequence is operably linked to SEQ ID NO: 1, SEQ ID NO: 2, SEQ ID NO: 3, or SEQ ID NO: 4. In another more specific embodiment, this vector comprises an IRES. In another specific embodiment, the invention relates to a vector comprising SEQ ID NO: 1, SEQ ID NO: 2, SEQ ID NO: 3, or SEQ ID NO: 4 or a subsequence thereof, and an MCS, wherein when a coding sequence is present within the MCS, the coding sequence is operably linked to the nucleotide sequence of SEQ ID NO: 1, SEQ ID NO: 2, SEQ ID NO: 3, or SEQ ID NO: 4 or a subsequence thereof. In another specific embodiment, the invention relates to a vector comprising SEQ ID NO: 1, SEQ ID NO: 2, SEQ ID NO: 3, or SEQ ID NO: 4 or a subsequence thereof, and an MCS, wherein when a coding sequence is present within the MCS, the coding sequence is operably linked to the transcription activating sequence of SEQ ID NO: 1, SEQ ID NO: 2, SEQ ID NO: 3, or SEQ ID NO: 4 or a subsequence thereof. In another specific embodiment, any of the above the vectors contains a coding sequence within the MCS. In another specific embodiment, any of the above vectors that contains a coding sequence, said coding sequence is a reporter gene sequence. In a more specific embodiment, said reporter gene sequence encodes β-galactosidase, a fluorescent protein, chloramphenicol acetyltransferase, luciferase or an antigenic marker. In another specific embodiment, said coding sequence is a neuroprotective sequence. In another specific embodiment, the invention provides a vector comprising a promoter and an MCS operably linked in an upstream-to-downstream order, and the nucleotide sequence of SEQ ID NO: 1, SEQ ID NO: 2, SEQ ID NO: 3, or SEQ ID NO: 4 or a transcription activating nucleotide sequence thereof. In a more specific embodiment, this vector further comprises an internal ribosomal entry site (IRES).

Any of the vectors of the invention can be adapted for transfer to a eukaryotic host cell, including a human host cell. In a more specific embodiment, the eukaryotic host cell is a nervous system cell. In a more specific embodiment, the nervous system cell is a nervous system cell line, glial cell, astrocyte, oligodendrocyte, mesencephalic neuron, hypothalamic neuron or cortical neuron. In another embodiment, the vectors above are adapted for transfer to a prokaryotic host cell.

The invention further provides for host cells, or progeny thereof, containing the vectors above. In a more specific embodiment, said host cell is a eukaryotic cell, including a human host cell. In a more specific embodiment, said host cell is a nervous system cell. In another specific embodiment, said host cell is a prokaryotic cell.

The invention also provides for kits containing one or more of the vectors and or host cells of the invention in one or more containers, and, preferably, further containing instructions for use.

The present invention also relates to transgenic non-human animals engineered to contain a nucleic acid regulatory sequence of the invention. The nucleic acid regulatory sequence can be contained within an episome or, alternatively, the sequence can be integrated within the genome of the transgenic animal. Genomic insertion can be by either homologous or non-homologous recombination.

The invention further provides a method of expressing a coding sequence in a host cell in cell culture. In one embodiment, the method comprises culturing a host cell containing a vector of the invention that contains a coding sequence under conditions effective to allow expression of the coding sequence by said host cell. In another embodiment, the method comprises culturing a host cell of the invention wherein the nucleic acid regulatory sequence controls expression of a coding sequence endogenously present in the genome of said host cell, under conditions effective to allow expression of the coding sequence by said host cell.

The invention also provides a method of producing a peptide or polypeptide comprising maintaining a host cell of the invention that contains a coding sequence that encodes a peptide or polypeptide under conditions effective to allow expression of said coding sequence, and to allow translation of the resulting mRNA, such that a peptide or polypeptide is expressed. In one embodiment, the coding sequence is present as part of a vector of the invention. In another embodiment, the host cell has been engineered such that a nucleic acid regulatory sequence of the invention controls expression of a coding sequence endogenously present in the genome of said host cell, under conditions effective to allow expression of the coding sequence by said host cell. In a more specific embodiment, the vector is present in the genome of said host cell.

The invention also provides a method of identifying a modulator of a nucleic acid regulatory sequence of the invention, comprising: (a) contacting a host cell containing a nucleic acid regulatory sequence of the invention operably linked to a reporter gene sequence with a test compound; and (b) assaying expression of the reporter gene, such that, if a change in reporter gene expression relative to its expression in the absence of the test compound, is detected, a modulator of the nucleic acid regulatory sequence is identified. In a particular embodiment, the host cell is a nervous system cell.

As used herein, an “isolated nucleic acid” is a nucleic acid outside its normal biological context (i.e., outside an intact chromosome). The term is also not intended to refer to nucleotide sequences consisting of the sequences disclosed in GenBank accession numbers AP000555 and AE001301 (see Section 6.2), or in GenBank accession numbers AF240786 and Z84718 (see Section 6.3), or in GenBank accession numbers Z95114 and AC016021 (see Section 6.4), or in GenBank accession numbers AC000093 and AC008780 (see Section 6.5). Finally, the term “isolated nucleic acid” as used herein is also not intended to refer to any other full-length sequence disclosed in GenBank. Further, an isolated nucleic acid molecule of the invention contains no more than up to about 5,000 to 10,000 nucleotides of sequence that would endogenously flank SEQ ID NO: 1, or SEQ ID NO: 2, or SEQ ID NO: 3, or SEQ ID NO: 4. Additionally, the term refers to either the single-stranded or double-stranded form of the nucleic acid molecule. Furthermore, the isolated nucleic acid molecule may consist of DNA or RNA, and may contain base analogs.

A “nucleic acid regulatory sequence” or “regulatory sequence” comprises a nucleotide sequence that, when operably linked to a nucleic acid of interest, modulates (e.g., activates (promotes, enhances) or inhibits (suppresses, represses, silences) transcription) the nucleic acid of interest, particularly in a cell. A nucleotide sequence is considered “transcription activating” if, when operably linked to a nucleic acid whose expression may be monitored, and placed in a cell (e.g., a nervous system cell in cell culture) under conditions under which expression may take place, promotes or enhances the expression of the nucleic acid detectably above the expression of the same nucleic acid in the absence of the nucleotide sequence operably linked thereto. A nucleic acid regulatory sequence “promotes” transcription of a nucleic acid of interest if, when operably linked to the nucleic acid of interest, it elicits a detectable level of expression of the nucleic acid of interest. A nucleic acid regulatory sequence “enhances” transcription of a nucleic acid of interest if, when operably linked to the nucleic acid of interest, it increases the detectable level of expression relative to expression of the nucleic acid of interest in the absence of the nucleic acid regulatory sequence operably linked thereto. Generally, a nucleic acid regulatory sequence is considered to enhance transcription of the nucleic acid of interest when said nucleic acid is already expressed to some detectable level (e.g., is controlled by a promoter sequence) that is increased by the nucleic acid regulatory sequence.

A nucleotide sequence, e.g., a nucleic acid regulatory sequence, is “operably linked” to a nucleic acid of interest if said nucleotide sequence is present in a cis configuration relative to said nucleic acid of interest, i.e., the nucleotide sequence attached via a covalent linkage (e.g., a phosphodiester linkage) to the same nucleic acid molecule that comprises the nucleic acid of interest. In one embodiment, a nucleic acid regulatory sequence can be adjacent to a nucleic acid of interest or to a promoter sequence that promotes expression of the nucleic acid of interest. The nucleic acid regulatory sequence can be placed upstream (i.e., 5′) of the sequence whose expression is to be activated (promoted, enhanced) or inhibited. Additionally, in particular where the regulatory sequence has enhancer or silencer activity, the nucleic acid regulatory sequence can be placed within (e.g., in an intron) or downstream (i.e., 3′) of the sequence whose expression is to be modulated.

A “coding sequence” is a nucleotide sequence that, when transcribed, yields an RNA molecule. In a preferred embodiment, a coding sequence comprises an open reading frame (ORF) that can be translated into a peptide or polypeptide sequence. In another preferred embodiment, a coding sequence comprises a nucleotide sequence that, when transcribed, yields a tRNA, rRNA, antisense RNA or enzymatically active RNA molecule.

A first nucleic acid sequence is considered “heterologous” to a second nucleic acid sequence when the sequences are not endogenously present contiguous to each other, or when neither sequence is endogenously contained within the other.

A “vector” is any nucleic acid that is self-replicating in at least one host cell, and is capable of containing the isolated nucleic acid for storage, replication, or propagation of the isolated nucleic acid, or for expression of a coding sequence operably linked to the isolated nucleic acid.

A “nervous system cell” can refer to a cell of the central nervous system (CNS), such as neurons, e.g., cortical, hippocampal, mesencephalic or medullary neurons, and glia in the brain, as well as to eye, spinal cord, and olfactory bulb cells, and to cells in the peripheral nervous system (PNS).

A “peptide” refers to a macromolecule of from two to about nineteen amino acids covalently linked, e.g., covalently linked via peptide bonds.

A “polypeptide” refers to a macromolecule of at least about twenty amino acids covalently linked, e.g., covalently linked via peptide bonds.

### 4. BRIEF DESCRIPTION OF THE DRAWINGS

FIG. 1 is a diagram of the plasmid pCOGENT1 containing CNI-01054. Regulatory sequences of the present invention are indicated as “UNIQUE SEQUENCE CNI-01054”. pCOGENT1 contains a basal promoter (i.e., a TATA box) between the BamHI and ClaI sites. The negative control plasmid for expression experiments is pCOGENT1 containing only a basal promoter and no regulatory sequence insert. The positive control is pCOGENT1(E), which is pCOGENT1 containing the CMV enhancer region inserted into the MCS upstream of the basal promoter.

FIG. 2 depicts the DNA sequence of CNI-01054.

FIG. 3 depicts the UCSC linkage map of a region of human chromosome 22 containing the nucleotide sequence of CNI-01054. The position of the sequence of CNI-01054 in the map is the complement of base positions 18803052 to 18803240. The position in the UCSC linkage map (University of California-Santa Cruz Oct. 7, 2000 freeze) corresponds to the complement of positions 5728083 to 5728270 in the nucleotide sequence of human chromosome 22 as reported in CHR22_{—}19_{—}05_{—}2000 version 2 (The Sanger Centre, Cambridge, England). Base Position: Chromosomal coordinates, numbered from the telomere of the short arm of human chromosome 22. Chromosome Band: Light and dark blocks show traditional cytological bands seen with Giemsa staining. STS Markers: Location of markers from genetic, RH, YAC, and FISH maps. Mouse Synteny: Syntenic chromosomal region in mouse if known. GC Percent: Darker shades of gray correspond to higher % GC figured for a window of 20 kbp. FPC Contigs: Large dark blocks correspond to fingerprint map contigs at the Washington University (Genome Sequencing Center, Washington University School of Medicine, St. Louis, Mo.). Assembly: An individual box shows the assembly extracted from a single clone fragment. Gaps in the path appear as white space between the boxes at an adequate zoom level, with a thin horizontal line connecting boxes bridged over a gap. Gap: Shows locations of gaps in the assembly with black boxes or vertical lines. Small gaps may have artefactually coalesced in the graphic. Gaps spanned by mRNA and paired reads have a white horizontal line through the black box to indicate bridging. Coverage: In dense display, the level of gray gives level of coverage: White/Clear: no coverage (gap); Light Gray: predraft (less than 4× shotgun); Medium Gray: draft (at least 4× shotgun); Dark Gray: multiple draft, covered by more than one draft clone; Finished: covered by a finished clone. YourSeq: Position of the DNA sequence of CNI-01054 relative to other sequences or features in the linkage map. Known Genes (from full length mRNAs): Known protein coding genes from LocusLink. Exons are represented by black boxes; thin horizontal lines represent introns. In the full view, the arrows on the introns indicate direction of transcription. Affymetrix Gene Predictions (excluding known genes): Gene predictions based on ESTs, mRNA, codon usage, and splice site statistics by the proprietary program Genie, supplied by David Kulp at Affymetrix. Ensembl Genes: Gene predictions from Ensembl, an automatic DNA sequence assembly and tracking system developed by the European Bioinformatics Institute and the Sanger Center (Cambridgeshire, Wellcome Trust Genome Campus, UK. Arrows on the introns, when present, indicate direction of transcription. Fgenesh++ Gene Predictions: Fgenesh++ predictions based on Softberry's gene finding software (Salamov & Solovyev, Genome Research 10(5):516-522 (2000)). Full mRNAs: Shows aligning regions as black boxes or vertical lines connected by horizontal lines for gaps. In full display, arrows on the introns indicate the direction of transcription. Human ESTs That Have Been Spliced: Shows spliced human ESTs. This track may suggest alternative splicings. Human ESTs: In dense mode the level of gray in this track represents the number of ESTs that align at that region. RNA Genes: Indicated location of non-protein coding RNA genes and pseudogenes, including tRNAs, rRNAs, SRPs, C/D box methylation guide snoRNAs, U6 etc. RNA-like, HBII, and hVS-like elements.

FIG. 4 shows the locations of transcription-factor binding motifs in CNI-01054. The names of the factors that bind the motifs are displayed above the diagram. The nucleotide position of the motifs as they occur sequentially 5′ to 3′ in the nucleotide sequence of CNI-01054 are indicated to the right of the transcription factor name.

FIG. 5 depicts quantitative measurement of cell transfection in coronal brain slices prepared from three regions along the rostral-caudal axis. Cells were transfected with pCOGENT1 containing CNI-01054, negative control DNA, or positive control DNA. The number of cells expressing the reporter gene in each slice is determined visually.

FIG. 6A shows an image of a coronal brain slice transfected with pCOGENT1 containing CNI-01054.

FIG. 6B shows an image of a coronal brain slice transfected with a negative control plasmid, pCOGENT1.

FIG. 6C shows an image of a coronal brain slice transfected with positive control DNA, pCOGENT(E).

FIG. 7 is a diagram of the plasmid pCOGENT1 containing CNI-01056. Regulatory sequences of the present invention are indicated as “UNIQUE SEQUENCE CNI-01056”. pCOGENT1 contains a basal promoter (i.e., a TATA box) between the BamHI and ClaI sites. The negative control plasmid for expression experiments is pCOGENT1 containing only a basal promoter and no regulatory sequence insert; the positive control is pCOGENT1(E), which is pCOGENT1 containing the CMV enhancer region inserted into the MCS upstream of the basal promoter.

FIG. 8 depicts the DNA sequence of CNI-01056.

FIG. 9 depicts the UCSC linkage map of a region of human chromosome 22 containing the nucleotide sequence of CNI-01056. The the sequence of CNI-01056 occurs in two locations in the map: at base positions 20968063 to 20968367, and on the complementary strand at base positions 20949437 to 20949741. The position in the UCSC linkage map (University of California-Santa Cruz Oct. 7, 2000 freeze) correspond, respectively, to positions 7893094 to 7893397, and 7874468 to 7874771 in the nucleotide sequence of human chromosome 22 as reported in CHR22_{—}19_{—}05_{—}2000 version 2 (The Sanger Centre, Cambridge, England). Base Position: Chromosomal coordinates, numbered from the telomere of the short arm of human chromosome 22. Chromosome Band: Light and dark blocks show traditional cytological bands seen with Giemsa staining. STS Markers: Location of markers from genetic, RH, YAC, and FISH maps. Mouse Synteny: Syntenic chromosomal region in mouse if known. GC Percent: Darker shades of gray correspond to higher % GC figured for a window of 20 kbp. FPC Contigs: Large dark blocks correspond to fingerprint map contigs at the Washington University (Genome Sequencing Center, Washington University School of Medicine, St. Louis, Mo.). Assembly: An individual box shows the assembly extracted from a single clone fragment. Gaps in the path appear as white space between the boxes at an adequate zoom level, with a thin horizontal line connecting boxes bridged over a gap. Gap: Shows locations of gaps in the assembly with black boxes or vertical lines. Small gaps may have artefactually coalesced in the graphic. Gaps spanned by mRNA and paired reads have a white horizontal line through the black box to indicate bridging. Coverage: In dense display, the level of gray gives level of coverage: White/Clear: no coverage (gap); Light Gray: predraft (less than 4× shotgun); Medium Gray: draft (at least 4× shotgun); Dark Gray: multiple draft, covered by more than one draft clone; Finished: covered by a finished clone. YourSeq: Position of the DNA sequence of CNI-01056 relative to other sequences or features in the linkage map. Known Genes (from full length mRNAs): Known protein coding genes from LocusLink. Exons are represented by black boxes; thin horizontal lines represent introns. In the full view, the arrows on the introns indicate direction of transcription. Affymetrix Gene Predictions (excluding known genes): Gene predictions based on ESTs, mRNA, codon usage, and splice site statistics by the proprietary program Genie, supplied by David Kulp at Affymetrix. Ensembl Genes: Gene predictions from Ensembl, an automatic DNA sequence assembly and tracking system developed by the European Bioinformatics Institute and the Sanger Center (Cambridgeshire, Wellcome Trust Genome Campus, UK. Arrows on the introns, when present, indicate direction of transcription. Fgenesh++ Gene Predictions: Fgenesh++ predictions based on Softberry's gene finding software (Salamov & Solovyev, Genome Research 10(5):516-522 (2000)). Full mRNAs: Shows aligning regions as black boxes or vertical lines connected by horizontal lines for gaps. In full display, arrows on the introns indicate the direction of transcription. Human ESTs That Have Been Spliced: Shows spliced human ESTs. This track may suggest alternative splicings. Human ESTs: In dense mode the level of gray in this track represents the number of ESTs that align at that region. RNA Genes: Indicated location of non-protein coding RNA genes and pseudogenes, including tRNAs, rRNAs, SRPs, C/D box methylation guide snoRNAs, U6 etc. RNA-like, HBII, and hVS-like elements.

FIG. 10 shows the locations of transcription-factor binding motifs in CNI-01056. The names of the factors that bind the motifs are displayed above the diagram. The nucleotide position of the motifs as they occur sequentially 5′ to 3′ in the nucleotide sequence of CNI-01056 are indicated to the right of the transcription factor name.

FIG. 11 depicts quantitative measurement of cell transfection in coronal brain slices prepared from three regions along the rostral-caudal axis. Cells were transfected with pCOGENT1 containing CNI-01056, negative control DNA, or positive control DNA. The number of cells expressing the reporter gene in each slice is determined visually.

FIG. 12A shows an image of a coronal brain slice transfected with pCOGENT1 containing CNI-01056.

FIG. 12B shows an image of a coronal brain slice transfected with a negative control plasmid, pCOGENT1.

FIG. 12C shows an image of a coronal brain slice transfected with positive control DNA, pCOGENT(E).

FIG. 13 is a diagram of the plasmid pCOGENT1 containing CNI-01058. Regulatory sequences of the present invention are indicated as “UNIQUE SEQUENCE CNI-01058”. pCOGENT1 contains a basal promoter (i.e., a TATA box) between the BamHI and ClaI sites. The negative control plasmid for expression experiments is pCOGENT1 containing only a basal promoter and no regulatory sequence insert; the positive control is pCOGENT1(E), which is pCOGENT1 containing the CMV enhancer region inserted into the MCS upstream of the basal promoter.

FIG. 14 depicts the DNA sequence of CNI-01058.

FIG. 15 depicts the UCSC linkage map of a region of human chromosome 22 containing the nucleotide sequence of CNI-01058. The position of the sequence of CNI-01058 in the map is from base position 33139028 to 33139535. The position in the UCSC linkage map (University of California-Santa Cruz Oct. 7, 2000 freeze) corresponds to positions 20031300 to 20031807 in the nucleotide sequence of human chromosome 22 as reported in CHR22_{—}19_{—}05_{—}2000 version 2 (The Sanger Centre, Cambridge, England). Base Position: Chromosomal coordinates, numbered from the telomere of the short arm of human chromosome 22. Chromosome Band: Light and dark blocks show traditional cytological bands seen with Giemsa staining. STS Markers: Location of markers from genetic, RH, YAC, and FISH maps. Mouse Synteny: Syntenic chromosomal region in mouse if known. GC Percent: Darker shades of gray correspond to higher % GC figured for a window of 20 kbp. FPC Contigs: Large dark blocks correspond to fingerprint map contigs at the Washington University (Genome Sequencing Center, Washington University School of Medicine, St. Louis, Mo.). Assembly: An individual box shows the assembly extracted from a single clone fragment. Gaps in the path appear as white space between the boxes at an adequate zoom level, with a thin horizontal line connecting boxes bridged over a gap. Gap: Shows locations of gaps in the assembly with black boxes or vertical lines. Small gaps may have artefactually coalesced in the graphic. Gaps spanned by mRNA and paired reads have a white horizontal line through the black box to indicate bridging. Coverage: In dense display, the level of gray gives level of coverage: White/Clear: no coverage (gap); Light Gray: predraft (less than 4× shotgun); Medium Gray: draft (at least 4× shotgun); Dark Gray: multiple draft, covered by more than one draft clone; Finished: covered by a finished clone. YourSeq: Position of the DNA sequence of CNI-01058 relative to other sequences or features in the linkage map. Known Genes (from full length mRNAs): Known protein coding genes from LocusLink. Exons are represented by black boxes; thin horizontal lines represent introns. In the full view, the arrows on the introns indicate direction of transcription. Affymetrix Gene Predictions (excluding known genes): Gene predictions based on ESTs, mRNA, codon usage, and splice site statistics by the proprietary program Genie, supplied by David Kulp at Affymetrix. Ensembl Genes: Gene predictions from Ensembl, an automatic DNA sequence assembly and tracking system developed by the European Bioinformatics Institute and the Sanger Center (Cambridgeshire, Wellcome Trust Genome Campus, UK. Arrows on the introns, when present, indicate direction of transcription. Fgenesh++ Gene Predictions: Fgenesh++ predictions based on Softberry's gene finding software (Salamov & Solovyev, Genome Research 10(5):516-522 (2000)). Full mRNAs: Shows aligning regions as black boxes or vertical lines connected by horizontal lines for gaps. In full display, arrows on the introns indicate the direction of transcription. Human ESTs That Have Been Spliced: Shows spliced human ESTs. This track may suggest alternative splicings. Human ESTs: In dense mode the level of gray in this track represents the number of ESTs that align at that region. RNA Genes: Indicated location of non-protein coding RNA genes and pseudogenes, including tRNAs, rRNAs, SRPs, C/D box methylation guide snoRNAs, U6 etc. RNA-like, HBII, and hVS-like elements.

FIG. 16 shows the locations of transcription-factor binding motifs in CNI-01058. The names of the factors that bind the motifs are displayed above the diagram. The nucleotide position of the motifs as they occur sequentially 5′ to 3′ in the nucleotide sequence of CNI-01058 are indicated to the right of the transcription factor name.

FIG. 17 depicts quantitative measurement of cell transfection in coronal brain slices prepared from three regions along the rostral-caudal axis. Cells were transfected with pCOGENT1 containing CNI-01058, negative control DNA, or positive control DNA. The number of cells expressing the reporter gene in each slice is determined visually.

FIG. 18A shows an image of a coronal brain slice transfected with pCOGENT1 containing CNI-01058.

FIG. 18B shows an image of a coronal brain slice transfected with a negative control plasmid, pCOGENT1.

FIG. 18C shows an image of a coronal brain slice transfected with positive control DNA, pCOGENT(E).

FIG. 19 is a diagram of the plasmid pCOGENT1 containing CNI-01059. Regulatory sequences of the present invention are indicated as “UNIQUE SEQUENCE CNI-01059”. pCOGENT1 contains a basal promoter (i.e., a TATA box) between the BamHI and ClaI sites. The negative control plasmid for expression experiments is pCOGENT1 containing only a basal promoter and no regulatory sequence insert; the positive control is pCOGENT1(E), which is pCOGENT1 containing the CMV enhancer region inserted into the MCS upstream of the basal promoter.

FIG. 20 depicts the DNA sequence of CNI-01059.

FIG. 21 depicts the UCSC linkage map of a region of human chromosome 22 containing the nucleotide sequence of CNI-01059. The position of the sequence of CNI-01059 in the map is from base position 16662707 to 16666454. The position in the UCSC linkage map (University of California-Santa Cruz Oct. 7, 2000 freeze) corresponds to positions 3588115 to 3591861 in the nucleotide sequence of human chromosome 22 as reported in CHR22_{—}19_{—}05_{—}2000 version 2 (The Sanger Centre, Cambridge, England). Base Position: Chromosomal coordinates, numbered from the telomere of the short arm of human chromosome 22. Chromosome Band: Light and dark blocks show traditional cytological bands seen with Giemsa staining. STS Markers: Location of markers from genetic, RH, YAC, and FISH maps. Mouse Synteny: Syntenic chromosomal region in mouse if known. GC Percent: Darker shades of gray correspond to higher % GC figured for a window of 20 kbp. FPC Contigs: Large dark blocks correspond to fingerprint map contigs at the Washington University (Genome Sequencing Center, Washington University School of Medicine, St. Louis, Mo.). Assembly: An individual box shows the assembly extracted from a single clone fragment. Gaps in the path appear as white space between the boxes at an adequate zoom level, with a thin horizontal line connecting boxes bridged over a gap. Gap: Shows locations of gaps in the assembly with black boxes or vertical lines. Small gaps may have artefactually coalesced in the graphic. Gaps spanned by mRNA and paired reads have a white horizontal line through the black box to indicate bridging. Coverage: In dense display, the level of gray gives level of coverage: White/Clear: no coverage (gap); Light Gray: predraft (less than 4× shotgun); Medium Gray: draft (at least 4× shotgun); Dark Gray: multiple draft, covered by more than one draft clone; Finished: covered by a finished clone. YourSeq: Position of the DNA sequence of CNI-01059 relative to other sequences or features in the linkage map. Known Genes (from full length mRNAs): Known protein coding genes from LocusLink. Exons are represented by black boxes; thin horizontal lines represent introns. In the full view, the arrows on the introns indicate direction of transcription. Affymetrix Gene Predictions (excluding known genes): Gene predictions based on ESTs, mRNA, codon usage, and splice site statistics by the proprietary program Genie, supplied by David Kulp at Affymetrix. Ensembl Genes: Gene predictions from Ensembl, an automatic DNA sequence assembly and tracking system developed by the European Bioinformatics Institute and the Sanger Center (Cambridgeshire, Wellcome Trust Genome Campus, UK. Arrows on the introns, when present, indicate direction of transcription. Fgenesh++ Gene Predictions: Fgenesh++ predictions based on Softberry's gene finding software (Salamov & Solovyev, Genome Research 10(5):516-522 (2000)). Full mRNAs: Shows aligning regions as black boxes or vertical lines connected by horizontal lines for gaps. In full display, arrows on the introns indicate the direction of transcription. Human ESTs That Have Been Spliced: Shows spliced human ESTs. This track may suggest alternative splicings. Human ESTs: In dense mode the level of gray in this track represents the number of ESTs that align at that region. RNA Genes: Indicated location of non-protein coding RNA genes and pseudogenes, including tRNAs, rRNAs, SRPs, C/D box methylation guide snoRNAs, U6 etc. RNA-like, HBII, and hVS-like elements.

FIG. 22 shows the locations of transcription-factor binding motifs in CNI-01059. The names of the factors that bind the motifs are displayed above the diagram. The nucleotide position of the motifs as they occur sequentially 5′ to 3′ in the nucleotide sequence of CNI-01059 are indicated to the right of the transcription factor name.

FIG. 23 depicts quantitative measurement of cell transfection in coronal brain slices prepared from three regions along the rostral-caudal axis. Cells were transfected with pCOGENT1 containing CNI-01059, negative control DNA, or positive control DNA. The number of cells expressing the reporter gene in each slice is determined visually.

FIG. 24A shows an image of a coronal brain slice transfected with pCOGENT1 containing CNI-01059.

FIG. 24B shows an image of a coronal brain slice transfected with a negative control plasmid, pCOGENT1.

FIG. 24C shows an image of a coronal brain slice transfected with positive control DNA, pCOGENT(E).

### 5. DETAILED DESCRIPTION OF THE INVENTION

5.1. The Regulatory Sequences

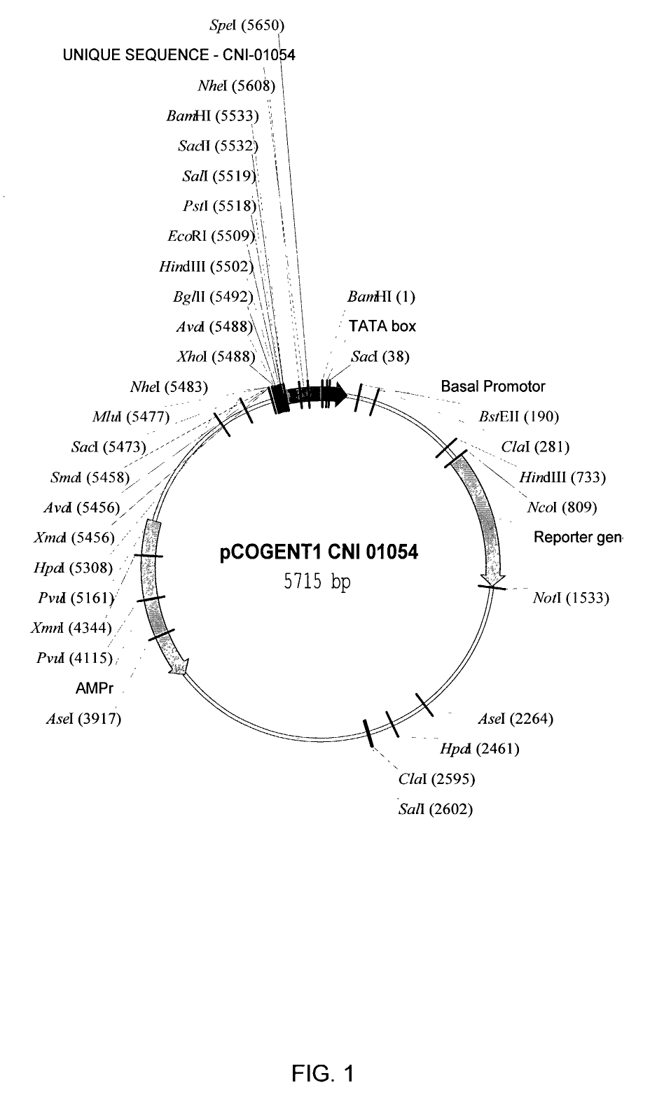

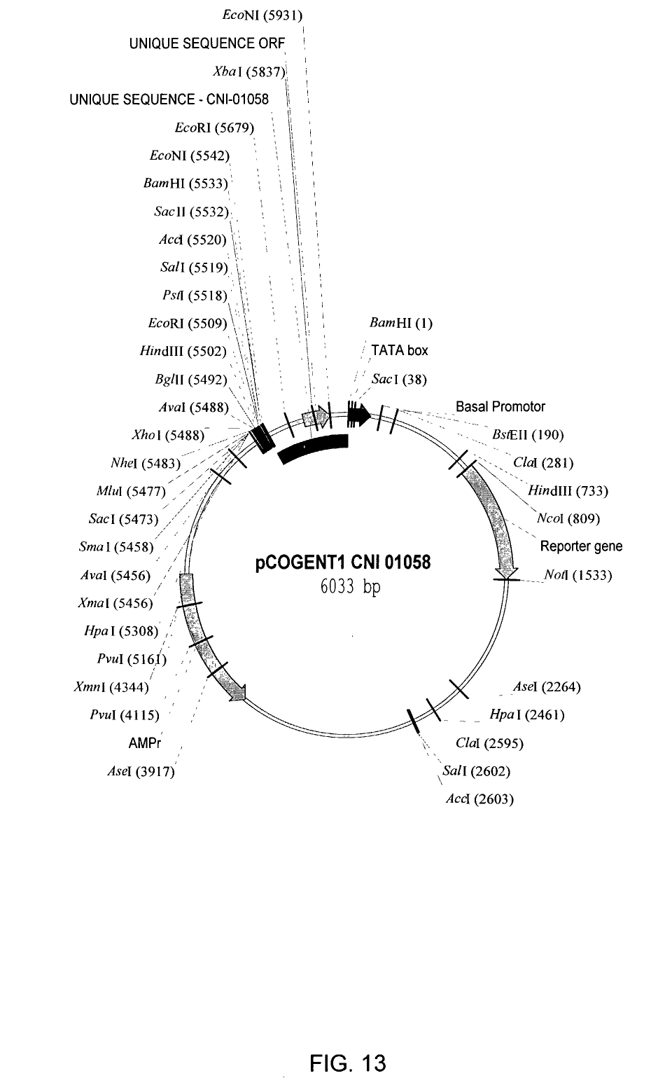

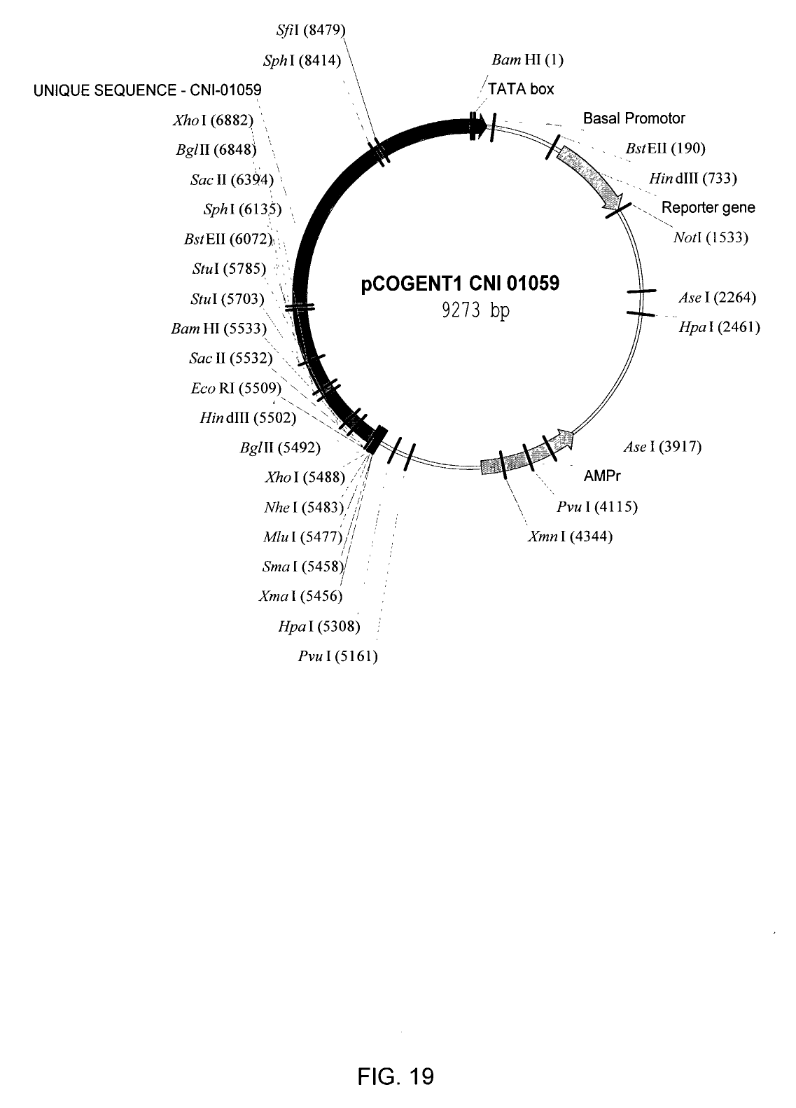

Using plasmid pCOGENT1 (FIG. 1, FIG. 7, FIG. 13, FIG. 19), sequences have been identified that modulate the expression of a reporter sequence in nervous system cells. The present invention therefore relates to the following nucleic acid molecules that represent nucleic acid regulatory sequence molecules of the invention:

TABLE 1
FULL-LENGTH REGULATORY NUCLEIC ACID MOLECULES

| REGULATORY | | | |
|---|---|---|---|
| SEQUENCE | SEQ ID NO: | FIG. NO. | LENGTH |
| CNI-01054 | 1 | 2 | 377 Nucleotides |
| CNI-01056 | 2 | 8 | 297 Nucleotides |
| CNI-01058 | 3 | 14 | 508 Nucleotides |
| CNI-01059 | 4 | 20 | 3747 Nucleotides |

In particular, SEQ ID NO: 1, and SEQ ID NO: 2, and SEQ ID NO: 3, and SEQ ID NO: 4 each promote or enhance gene expression in the nervous system.

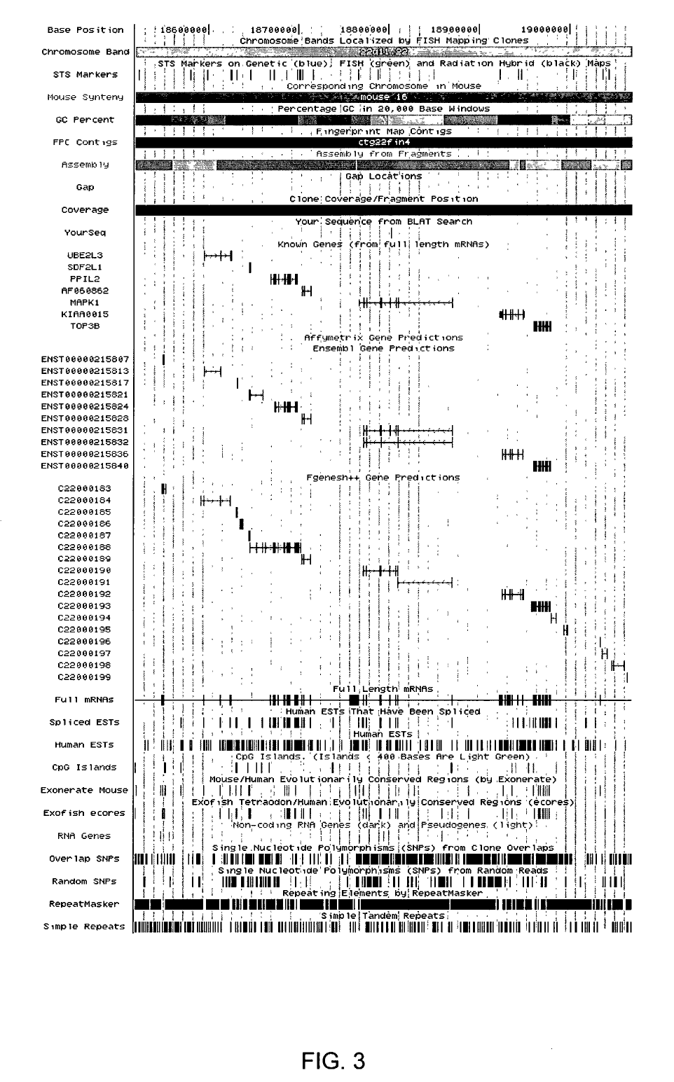

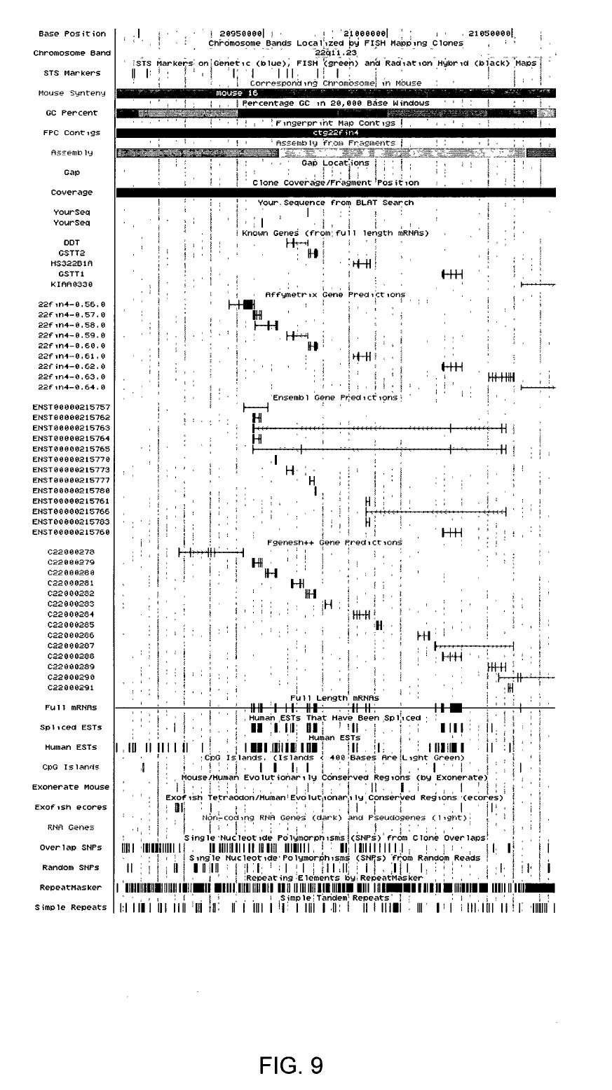

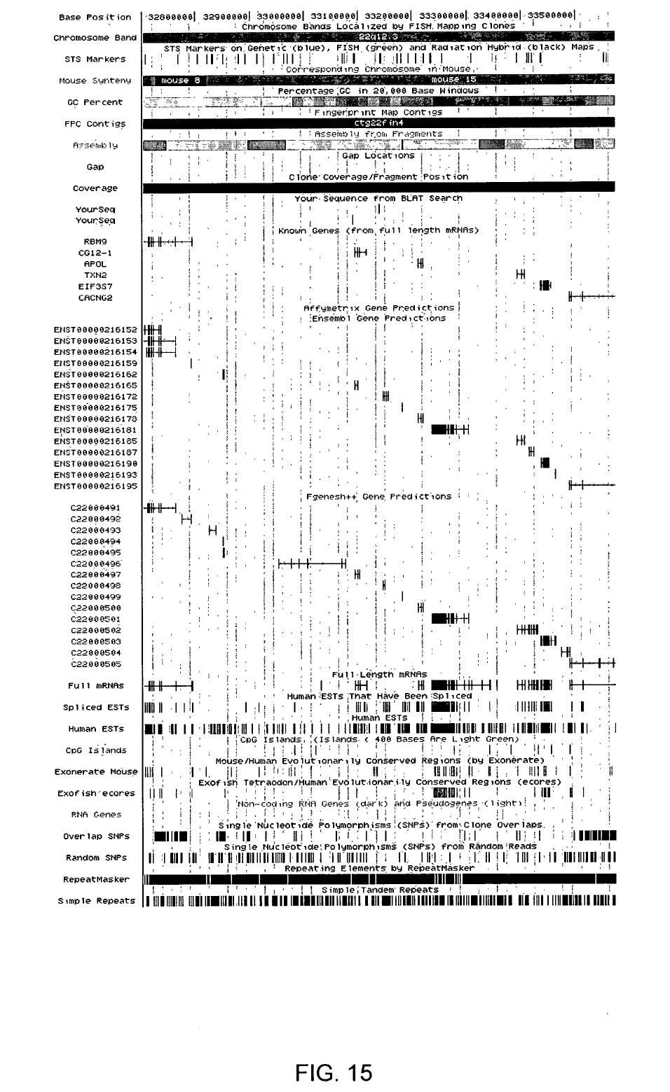

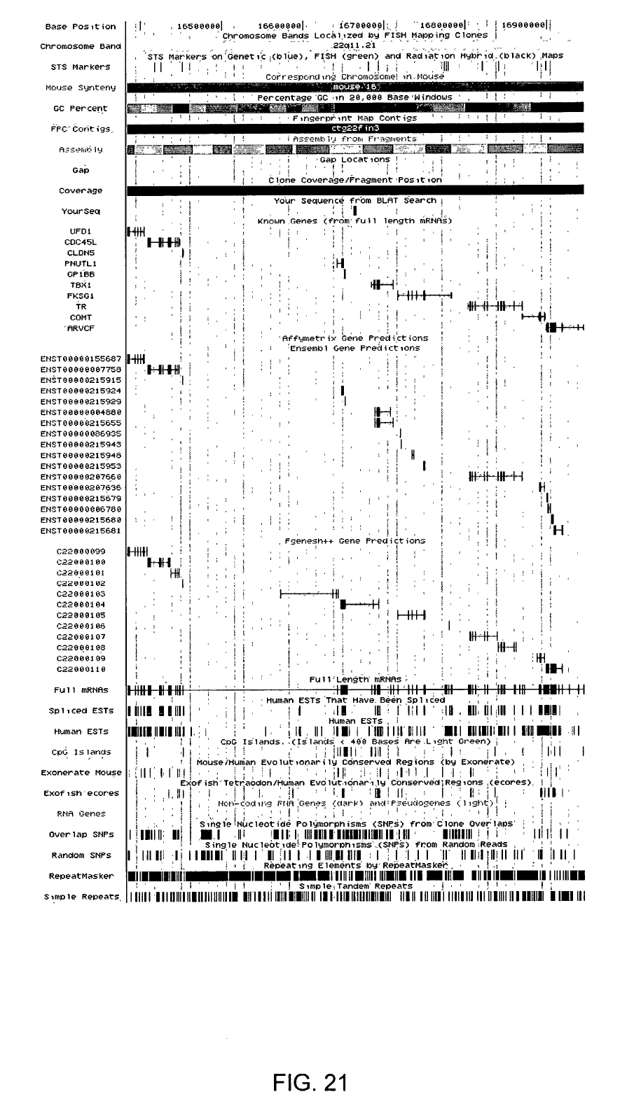

As depicted in FIG. 3 (UCSC linkage map of a region of human chromosome 22), the sequence of CNI-01054 is located within an intron of the gene encoding mitogen-activated protein kinase 1, also known as MAP kinase 1 (see Section 6.2). As depicted in FIG. 9 (UCSC linkage map of a region of human chromosome 22), the sequence of CNI-01056 is present in two locations in the sequence of human chromosome 22, each within approximately 20,000 bp of the other (see Section 6.3). As depicted in FIG. 15 (UCSC linkage map of a region of human chromosome 22), the nearest known or predicted gene to the sequence of CNI-01058 is a gene that is predicted to encode a form of human apolipoprotein L (see Section 6.4). As depicted in FIG. 21 (UCSC linkage map of a region of human chromosome 22), the sequence of CNI-01059 is located near three genes: peanut (Drosophila)-like 1 (PNUTL1); glycoprotein Ib, beta polypeptide (GP1BB); and T-box 1 (TBX1) (see Section 6.4).

The present invention also relates to isolated nucleic acid regulatory sequences comprising a transcription activating nucleotide sequence of SEQ ID NO: 1, SEQ ID NO: 2, SEQ ID NO: 3, SEQ ID NO: 4. Such nucleic acid regulatory sequences may be restriction fragments of the full-length sequences disclosed. For example, in one embodiment, the isolated nucleic acid regulatory sequence molecule is a restriction fragment of SEQ ID NO: 1. Thus, in a specific embodiment, a nucleic acid regulatory sequence of the invention the BamHI-BamHI fragment represented by nucleotides 1-195 of SEQ ID NO: 1. In another specific embodiment, a nucleic acid regulatory sequence of the invention is the BamHI-BamHI fragment represented by nucleotides 189-377 of SEQ ID NO: 1. In another specific embodiment, a nucleic acid regulatory sequence of the invention is the AccI-AccI fragment represented by nucleotides 14-208 of SEQ ID NO: 1. In another specific embodiment, a nucleic acid regulatory sequence of the invention is the Eco57I-Eco57I fragment represented by nucleotides 30-224 of SEQ ID NO: 1. In another specific embodiment, a nucleic acid regulatory sequence of the invention is the SpeI-SpeI fragment represented by nucleotides 118-312 of SEQ ID NO: 1. In another specific embodiment, a nucleic acid regulatory sequence of the invention is the AccI-BamHI fragment represented by nucleotides 14-195 of SEQ ID NO: 1. In another specific embodiment, a nucleic acid regulatory sequence of the invention is the BamHI-AccI fragment represented by nucleotides 202-377 of SEQ ID NO: 1. In another specific embodiment, a nucleic acid regulatory sequence of the invention is the Eco571-BamHI fragment represented by nucleotides 30-195 of SEQ ID NO: 1. In another specific embodiment, a nucleic acid regulatory sequence of the invention is the BamHI-Eco57I fragment represented by nucleotides 218-377 of SEQ ID NO: 1. In another specific embodiment, a nucleic acid regulatory sequence of the invention is the SpeI-BamHI fragment represented by nucleotides 118-195 of SEQ ID NO: 1. In another Specific embodiment, a nucleic acid regulatory sequence of the invention is the BamHI-SpeI fragment represented by nucleotides 306-377 of SEQ ID NO: 1.

In another embodiment the isolated nucleic acid regulatory sequence molecule is a restriction fragment of SEQ ID NO: 2. For example, in a specific embodiment, a nucleic acid regulatory sequence of the invention is, but is not limited to, the BspMI-BssHII fragment represented by nucleotides 20-295 of SEQ ID NO: 2. In another specific embodiment, a nucleic acid regulatory sequence of the invention is the Cfr10I-BssHII fragment represented by nucleotides 34-295 of SEQ ID NO: 2. In another specific embodiment, a nucleic acid regulatory sequence of the invention is the DraII-BssHII fragment represented by nucleotides 94-295 of SEQ ID NO: 2. In another specific embodiment, a nucleic acid regulatory sequence of the invention is the DraII-DraII fragment represented by nucleotides 94-273. In another specific embodiment, a nucleic acid regulatory sequence of the invention is the MspAII-BssHII fragment represented by nucleotides 122-295 of SEQ ID NO: 2. In another specific embodiment, a nucleic acid regulatory sequence of the invention is the BstMCI-BssHII fragment represented by nucleotides 140-295 of SEQ ID NO: 2. In another specific embodiment, a nucleic acid regulatory sequence of the invention is the Cfr10I-Cfr10I fragment represented by nucleotides 34-190 of SEQ ID NO: 2. In another specific embodiment, a nucleic acid regulatory sequence of the invention is the SfcI-BssHII fragment represented by nucleotides 149-295 of SEQ ID NO: 2. In another specific embodiment, a nucleic acid regulatory sequence of the invention is the MflI-BssHII fragment represented by nucleotides 163-295 of SEQ ID NO: 2. In another specific embodiment, a nucleic acid regulatory sequence of the invention is the BssAI-BssHII fragment represented by nucleotides 184-295 of SEQ ID NO: 2.

In another embodiment the isolated nucleic acid regulatory sequence molecule is a restriction fragment of SEQ ID NO: 3. For example, in a specific embodiment, a nucleic acid regulatory sequence of the invention is, but is not limited to, the XhoII-XhoII fragment represented by nucleotides 2-508 of SEQ ID NO: 3. In another specific embodiment, a nucleic acid regulatory sequence of the invention is the EcoNI-XhoII fragment represented by nucleotides 11-508 of SEQ ID NO: 3. In another specific embodiment, a nucleic acid regulatory sequence of the invention is the BseRI-XhoII fragment represented by nucleotides 13-508 of SEQ ID NO: 3. In another specific embodiment, a nucleic acid regulatory sequence of the invention is the Bpu1102I-XhoII fragment represented by nucleotides 18-508 of SEQ ID NO: 3. In another specific embodiment, a nucleic acid regulatory sequence of the invention is the AspHI-XhoII fragment represented by nucleotides 24-508 of SEQ ID NO: 3. In another specific embodiment, a nucleic acid regulatory sequence of the invention is the MslI-XhoII fragment represented by nucleotides 79-508 of SEQ ID NO: 3. In another specific embodiment, a nucleic acid regulatory sequence of the invention is the AlwNI-XhoII fragment represented by nucleotides 115-508. In another specific embodiment, a nucleic acid regulatory sequence of the invention is the XhoII-EcoNI fragment represented by nucleotides 2-406 of SEQ ID NO: 3. In another specific embodiment, a nucleic acid regulatory sequence of the invention is the EcoNI-EcoNI fragment represented by nucleotides 11-406 of SEQ ID NO: 3. In another specific embodiment, a nucleic acid regulatory sequence of the invention is the ApoI-XhoII fragment represented by nucleotides 148-508 of SEQ ID NO: 3.

In another embodiment the isolated nucleic acid regulatory sequence molecule is a restriction fragment of SEQ ID NO: 4. For example, in a specific embodiment, a nucleic acid regulatory sequence of the invention is, but is not limited to, the XhoII-XhoII fragment represented by nucleotides 1-3747 of SEQ ID NO: 4. In another specific embodiment, a nucleic acid regulatory sequence of the invention is the AspHI-XhoII fragment represented by nucleotides 29-3747 of SEQ ID NO: 4. In another specific embodiment, a nucleic acid regulatory sequence of the invention is the SfcI-XhoII fragment represented by nucleotides 42-3747 of SEQ ID NO: 4. In another specific embodiment, a nucleic acid regulatory sequence of the invention is the PstI-XhoII fragment represented by nucleotides 46-3747 of SEQ ID NO: 4. In another specific embodiment, a nucleic acid regulatory sequence of the invention is the BspMI-XhoII fragment represented by nucleotides 103-3747 of SEQ ID NO: 4. In another specific embodiment, a nucleic acid regulatory sequence of the invention is the Eco88I-XhoII fragment represented by nucleotides 106-3747 of SEQ ID NO: 4. In another specific embodiment, a nucleic acid regulatory sequence of the invention is the BpmI-XhoII fragment represented by nucleotides 117-3747 of SEQ ID NO: 4. In another specific embodiment, a nucleic acid regulatory sequence of the invention is the HindII-XhoII fragment represented by nucleotides 124-3747 SEQ ID NO: 4. In another specific embodiment, a nucleic acid regulatory sequence of the invention is the XhoII-AspHI fragment represented by nucleotides 1-3423 of SEQ ID NO: 4. In another specific embodiment, a nucleic acid regulatory sequence of the invention is the AspHI-AspHI fragment represented by nucleotides 29-3423 of SEQ ID NO: 4.

In the above examples the recited sequence ranges include the entire recognition sequence for each restriction enzyme. It will be clear to a person of skill in the art that other restriction fragments of SEQ ID NO: 1, SEQ ID NO: 2, SEQ ID NO: 3, OR SEQ ID NO: 4 may be generated using other restriction enzymes and used as regulatory sequences e.g., as transcription activating nucleic acid sequences. The above examples are not meant to limit the invention to any particular restriction fragment or fragments.

Nucleic acid regulatory sequences of the invention may also comprise part or all of the reverse compliment of the full-length sequences disclosed. In another embodiment, the nucleic acid regulatory sequence of the invention comprises an isolated nucleic acid molecule that is the reverse complement of the nucleotide sequence of SEQ ID NO: 1. In another embodiment, the nucleic acid regulatory sequence of the invention comprises an isolated nucleic acid molecule that is the reverse complement of the nucleotide sequence of SEQ ID NO: 2. In another embodiment, the nucleic acid regulatory sequence of the invention comprises an isolated nucleic acid molecule that is the reverse complement of the nucleotide sequence of SEQ ID NO: 3. In another embodiment, the nucleic acid regulatory sequence of the invention comprises an isolated nucleic acid molecule that is the reverse complement of the nucleotide sequence of SEQ ID NO: 4.

The invention also provides regulation sequences that comprise all or part of the reverse complement of SEQ ID 1, SEQ ID 2, SEQ ID 3, or SEQ ID 4. Thus, in another embodiment, the nucleic acid regulatory sequence of the invention comprises the reverse complement of the nucleotide sequence of a transcription activating sequence of SEQ ID NO: 1. In another embodiment, the nucleic acid regulatory sequence of the invention comprises the reverse complement of the nucleotide sequence of a transcription activating sequence of SEQ ID NO: 2. In another embodiment, the nucleic acid regulatory sequence of the invention comprises the reverse complement of the nucleotide sequence of a transcription activating sequence of SEQ ID NO: 3. In another embodiment, the nucleic acid regulatory sequence of the invention comprises the reverse complement of the nucleotide sequence of a transcription activating sequence of SEQ ID NO: 4. The transcription activating sequence may additionally be discrete fragments of the full-length sequences disclosed. For example, in a more specific embodiment, the transcription activating nucleotide sequence comprises at least about 20, 30, 40, 50, 75, 100, 200, 300, or 350 contiguous nucleotides of SEQ ID NO: 1 or the reverse complement thereof. For example, a nucleic acid regulatory sequence of the invention may be, but is not limited to, the nucleotide sequence nn₁-nn₂₀, mm₂₀-nn₄₀, nn₄₁-nn₆₀, nn₆₁-nn₈₀, nn₈₁-nn₁₀₀, nn₁₀₁-nn₁₂₀, nn₁₂₁-nn₁₄₀, nn₁₄₁-nn₁₆₀, nn₁₆₁-nn₁₈₀, nn₁₈₁-nn₂₀₀, nn₂₀₁-nn₂₂₀, nn₂₂₁-nn₂₄₀, nn₂₄₁-nn₂₆₀, nn₂₆₁-nn₂₈₀, nn₂₈₁-nn₃₀₀, nn₃₀₁-nn₃₂₀, nn₃₂₁-nn₃₄₀, nn₃₄₁-nn₃₆₀, nn₃₆₁-nn₃₇₇, or any contiguous combination thereof, of SEQ ID NO: 1 or the reverse complement of any of the foregoing. In this and in following examples, “nnₓ-nn_{y}” means nucleotide X to nucleotide Y of the specific SEQ ID NO. For example, nn₁-nn₂₀ of SEQ ID NO: 1 means contiguous nucleotides 1-20 of SEQ ID NO: 1. In this and in following examples “nnₓ-nn_{y}” means nucleotide X to nucleotide Y of the specific SEQ ID NO: 1. For example, “nucleotides nn₁-nn₂₀ of SEQ ID NO: 1” means contiguous nucleotides 1-20 of SEQ ID NO: 1. This format applies, of course, to subsequences of SEQ ID NO: 2, SEQ ID NO: 3 and SEQ ID NO: 4.

In another specific embodiment, the transcription activating sequence comprises at least about 20, 30, 40, 50, 75, 100, 200 or 250 contiguous nucleotides of SEQ ID NO: 2. For example, a nucleic acid regulatory sequence of the invention may be, but is not limited to, the nucleotide sequence nn₁-nn₂₀, nn₂₁-nn₄₀, nn₄₁-nn₆₀, nn₆₁-nn₈₀, nn₈₁-nn₁₀₀, nn₁₀₁-nn₁₂₀, nn₁₂₁-nn₁₄₀, nn₁₄₁-nn₁₆₀, nn₁₆₁-nn₁₈₀, nn₁₈₁-nn₂₀₀, nn₂₂₁-nn₂₄₀, nn₂₄₁-nn₂₆₀, nn₂₆₁-nn₂₈₀, nn₂₈₁-nn₃₀₀, nn₃₀₁-nn₃₂₀, nn₂₈₁-nn₃₀₀, nn₃₄₁-nn₃₄₀, nn₃₄₁-nn₃₆₀, or nn₃₆₁-nn₃₇₇, or any contiguous combination thereof, of SEQ ID NO: 2 or the reverse complement thereof.

In another specific embodiment, the transcription activating nucleotide sequence comprises at least about 20, 30, 40, 50, 75, 100, 200, 300, 400, or 500 contiguous nucleotides of SEQ ID NO: 3 or the reverse complement thereof. For example, in a specific embodiment, a nucleic acid regulatory sequence of the invention is, but is not limited to, the nucleotide sequence nn₁-nn₅₀, nn₅₁-nn₁₀₀, nn₁₀₁-nn₁₅₀, nn₁₅₁-nn₂₀₀, nn₂₀₁-nn₂₅₀, nn₂₅₁-nn₃₀₀, nn₃₀₁-nn₃₅₀, nn₃₅₁-nn₄₀₀, nn₄₀₁-nn₄₅₀, or nn₄₅₁-nn₅₀₈, or any contiguous combination thereof, of SEQ ID NO: 3 or the reverse complement thereof.

In another specific embodiment, the transcription activating nucleotide sequence comprises at least about 20, 30, 40, 50, 75, 100, 200, 300, 400, 500, 750, 1000, 2000, 3000, or 3500 contiguous nucleotides of SEQ ID NO: 4 or the reverse complement thereof. For example, in a specific embodiment, a nucleic acid regulatory sequence of the invention is, but is not limited to, the nucleotide sequence nn₁-nn₁₀₀, nn₁₀₁-nn₂₀₀, nn₂₀₁-nn₃₀₀, nn₃₀₁-nn₄₀₀, nn₄₀₁-nn₅₀₀, nn₅₀₁-nn₆₀₀, nn₆₀₁-nn₇₀₀, nn₇₀₁-nn₈₀₀, nn₈₀₁-nn₉₀₀, nn₉₀₁-nn₁₀₀₀, nn₁₀₀₁-nn₁₁₀₀, nn₁₁₀₁-nn₁₂₀₀, nn₁₂₀₁-nn₁₃₀₀, nn₁₃₀₁-nn₁₄₀₀, nn₁₄₀₁-nn₁₅₀₀, nn₁₅₀₁-nn₁₆₀₀, nn₁₆₀₁-nn₁₇₀₀, nn₁₇₀₁-nn₁₈₀₀, nn₁₈₀₁-nn₁₉₀₀, nn₁₉₀₁-nn₂₀₀₀, nn₂₀₀₁-nn₂₁₀₀, nn₂₁₀₁-nn₂₂₀₀, nn₂₂₀₁-nn₂₃₀₀, nn₂₃₀₁-nn₂₄₀₀, nn₂₄₀₁-nn₂₅₀₀, nn₂₅₀₁-nn₂₆₀₀, nn₂₆₀₁-nn₂₇₀₀, nn₂₇₀₁-nn₂₈₀₀, nn₂₈₀₁-nn₂₉₀₀, nn₂₉₀₁-nn₃₀₀₀, nn₃₀₀₁-nn₃₁₀₀, nn₃₁₀₁-nn₃₂₀₀, nn₃₂₀₁-nn₃₃₀₀, nn₃₃₀₁-nn₃₄₀₀, nn₃₄₀₁-nn_{3500, nn}₃₅₀₁-nn₃₆₀₀, nn₃₆₀₁-nn₃₇₀₀, or nn₃₇₀₁-nn₃₇₄₇, or any contiguous combination thereof, of SEQ ID NO: 4 or the reverse complement thereof.

It will be readily apparent to one of skill in the art that one can derive transcription activating nucleotide sequence of different lengths in a like manner for SEQ ID NO: 1, SEQ ID NO: 2, SEQ ID NO: 3, or SEQ ID NO: 4, where the sequence is at least 20, 30, 40, 50, 75, 100, or 200 nucleotides in length.

In another embodiment, the invention provides for sequences that hybridize to the full-length sequences or reverse complements thereof. For example, in specific embodiment, the invention provides for an isolated nucleic acid regulatory sequence molecule comprising a transcription nucleotide sequence that hybridizes over its entire length to the nucleotide sequence of SEQ ID NO: 1, to a transcriptional activating sequence of SEQ ID NO: 1, or to a complement or reverse complement thereof. In another embodiment, the invention provides for an isolated nucleic acid regulatory sequence molecule comprising a transcription activating nucleotide sequence that hybridizes over its entire length to the nucleotide sequence of SEQ ID NO: 2, to a transcriptional activating sequence of SEQ ID NO: 2, or to a complement or reverse complement thereof. In another embodiment, the invention provides for an isolated nucleic acid regulatory sequence molecule comprising a transcription activating nucleotide sequence that hybridizes over its entire length to the nucleotide sequence of SEQ ID NO: 3, to a transcriptional activating sequence of SEQ ID NO: 3, or to a complement or reverse complement thereof. In another embodiment, the invention provides for an isolated nucleic acid regulatory sequence molecule comprising a transcription activating nucleotide sequence that hybridizes over its entire length to the nucleotide sequence of SEQ ID NO: 4, to a transcriptional activating sequence of SEQ ID NO: 4, or to a complement or reverse complement thereof. The restriction fragments and discrete subsequences enumerated above represent sequences that hybridize along their entire lengths to the disclosed full-length sequences or their complements.

Hybridizing conditions can be of low or high stringency. Such stringency conditions are well known to those of skill in the art. By way of example and not limitation, sequences that hybridize under low stringency conditions are ones that would hybridize under conditions as follows (see also Shilo and Weinberg, Proc. Natl. Acad. Sci. U.S.A. 78:6789-6792 (1981)): Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg g/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and re-exposed to film.

Likewise, by way of example and not limitation, sequences that hybridize under highly stringent conditions are ones that hybridize under such conditions of high stringency as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Hybridization conditions are said to be “highly stringent” or “high stringency” when said conditions are at least as stringent as those disclosed in this paragraph.

Stringency can also be determined by calculating the Tm of the hybridization. Among the nucleic acid molecules of the invention are deoxyoligonucleotides (“oligos”) which hybridize under highly stringent or moderately stringent conditions to the nucleic acid molecules described above. In general, for probes between 14 and 70 nucleotides in length the melting temperature (Tm) is calculated using the formula: Tm (° C.)=81.5+16.6(log[monovalent cations (molar)])+0.41 (% G+C)−(500/N) where N is the length of the probe. If the hybridization is carried out in a solution containing formamide, the melting temperature is calculated using the equation Tm (° C.)=81.5+16.6(log[monovalent cations (molar)])+0.41 (% G+C)−(0.61% formamide)−(500/N) where N is the length of the probe. In general, hybridization is carried out at about 20-25 degrees below Tm (for DNA-DNA hybrids) or 10-15 degrees below Tm (for RNA-DNA hybrids). For example, the Tm decreases approximately 1° C. for every 1% of base pairs that are mismatched. For hybrids shorter than 20 base pairs, the Tm decreases by approximately 5° C. for every mismatched base pair. Stringent conditions, therefore, are those where the hybridization temperature is Tm-25° C. (where the maximum hybridization rate is observed) to Tm-5° C. (maximum stringency).

Also encompassed within the scope of the invention are modifications of the regulatory nucleotide sequences of the invention that do not substantially affect their transcriptional activities. Such modifications include additions, deletions and substitutions. When operably linked to the coding region for a heterologous gene, such modifications of the 377 nucleotide CNI-01054 (SEQ ID NO: 1) regulatory sequence, the 297 nucleotide CNI-01056 (SEQ ID NO: 2) regulatory sequence, the 508 nucleotide CNI-01058 (SEQ ID NO: 3) regulatory sequence, the 3747 nucleotide CNI-01059 (SEQ ID NO: 4) regulatory sequence, or nucleic acid regulatory sequences thereof, are sufficient to modulate expression of the operatively linked heterologous gene in a cell.

The present invention also relates to the nucleic acid regulatory sequences of the invention operably linked to a nucleic acid molecule comprising a coding sequence. Thus, the invention also provides for the control of gene expression using modifications of CNI-01054 (SEQ ID NO: 1) or a nucleic acid regulatory sequence thereof that substantially alters the transcriptional promotion activities of CNI-01054. In one embodiment, the invention provides CNI-01054 sequences that act as stronger modulators than full-length CNI-01054. In another embodiment, the invention provides such sequences that are weaker promoters than CNI-01054. In yet another embodiment, the invention provides such sequences that act to suppress or depress the level of transcription below that of the full-length CNI-01054.

The invention also provides for the control of gene expression using modifications of CNI-01056 (SEQ ID NO: 2) or a nucleic acid regulatory sequence thereof that substantially alters the transcriptional promotion activities of CNI-01056. In one embodiment, the invention provides CNI-01056 sequences that act as stronger modulators than full-length CNI-01056. In another embodiment, the invention provides such sequences that are weaker promoters than CNI-01056. In yet another embodiment, the invention provides such sequences that act to suppress or depress the level of transcription below that of the full-length CNI-01056.

The invention also provides for the control of gene expression using modifications of CNI-01058 (SEQ ID NO: 3) or a nucleic acid regulatory sequence thereof that substantially alters the transcriptional promotion activities of CNI-01058. In one embodiment, the invention provides CNI-01058 sequences that act as stronger modulators than full-length CNI-01058. In another embodiment, the invention provides such sequences that are weaker promoters than CNI-01058. In yet another embodiment, the invention provides such sequences that act to suppress or depress the level of transcription below that of the full-length CNI-01058.

The invention also provides for the control of gene expression using modifications of CNI-01059 (SEQ ID NO: 4) or a nucleic acid regulatory sequence thereof that substantially alters the transcriptional promotion activities of CNI-01059. In one embodiment, the invention provides CNI-01059 sequences that act as stronger modulators than full-length CNI-01059. In another embodiment, the invention provides such sequences that are weaker promoters than CNI-01059. In yet another embodiment, the invention provides such sequences that act to suppress or depress the level of transcription below that of the full-length CNI-01059.

If a restriction map is generated, the determination of those regions of the nucleic acid regulatory sequences of the invention strongest in promoting or enhancing gene expression is a straightforward task. The region is first digested with restriction endonucleases that produce the desired fragments. Preferably, the restriction endonucleases are commercially available, and recognize six-nucleotide sequences. Preferably, too, these restriction endonucleases utilize sites that are also present in the MCS of an expression vector, to facilitate cloning the fragments in such a way that they are operably linked to a gene to be expressed, the level of expression of which indicates the strength of promotion or enhancement of gene expression. Typically, the region is segregated into subregions representing progressively longer deletions from the 5′ end, or from the 3′ end; internal sequences may be deleted, as well. In general, those fragments that result in the most production of gene product are the strongest promoters; those that produce the least above background are the weakest. This example is not meant to be limiting, as there are other means to generate fragments in order to map promoter, enhancer or silencer regions; for example, exonuclease digestion.

The same procedure may be used for regulatory sequence fragments created by exonuclease digestion. Typically, an exonuclease is contacted with the regulatory sequence, and treatment is allowed to continue for varying periods of time, thus generating fragments of various sizes. The fragments are size-separated, for example, on a sizing column or in an agarose gel. The fragments can then either be blunt-end ligated into an expression vector, or can be tailed with linkers to facilitate cloning into such a vector. The resulting constructs are then analyzed for insert sequence and for the insert's ability to promote expression of the reporter gene.

The ability of sequences or fragments of the regulatory sequences of the invention to promote or enhance transcription can be assessed in two kinds of plasmid vectors. In one vector, the regulatory sequence or subfragments thereof is cloned into a site, typically part of an MCS, that places the regulatory sequence upstream of, and operably linked to, a reporter gene whose expression can be monitored. The vector, prior to insertion of the regulatory sequence, has no promoter of its own that can drive expression of the reporter gene. Expression of the reporter sequence over that seen with a no-insert control indicates that the regulatory sequence acts as a promoter of transcription. A second vector contains a promoter operably linked to the reporter gene. Here, the putative regulatory sequence is inserted upstream of the promoter, again typically into an MCS. If there is additional increase of the reporter gene above that seen in a promoter-only control, the regulatory sequence has enhancer activity.

It will be apparent to those of skill in the art that the above two vectors may additionally be used to discover other regulatory sequences, for example, homologous or analogous regulatory sequences that drive expression in the nervous systems of other species. For example, one may design sets of primers based upon the nucleotide sequence of the regulatory sequence of the invention, and perform PCR under moderately-stringent conditions well known to those of skill in the art on genomic DNA derived from a non-human species. PCR products are then cloned directly into one of the above two vectors. PCR products driving expression in the vector containing a promoter operably linked to the reporter gene have enhancer activity, while PCR products driving expression in the promoterless vector have promoter activity.

Alterations in the regulatory sequences can be generated using a variety of chemical and enzymatic methods which are well known to those skilled in the art. For example, regions of the sequences defined by restriction sites can be deleted. Oligonucleotide-directed mutagenesis can be employed to alter the sequence in a defined way and/or to introduce restriction sites in specific regions within the sequence. Additionally, deletion mutants can be generated using DNA nucleases such as Bal31 or ExoIII and S1 nuclease. Progressively larger deletions in the regulatory sequences are generated by incubating the DNA with nucleases for increased periods of time (see Ausubel, et al., CURRENT PROTOCOLS FOR MOLECULAR BIOLOGY (1989), for a review of mutagenesis techniques).

The altered sequences are evaluated for their ability to direct expression of heterologous coding sequences in appropriate host cells, e.g., nervous system cells. It is within the scope of the present invention that any altered regulatory sequences which retain their ability to direct expression of a coding sequence be incorporated into recombinant expression vectors for further use.

The regulatory nucleic acid sequences of the invention can routinely be analyzed for the presence of transcription elements by various publicly available computer programs. Putative transcription elements are located, for example, by means of comparing the sequence to known or known consensus transcription factor binding sequences, and determining that the percent identity between the two is significant.

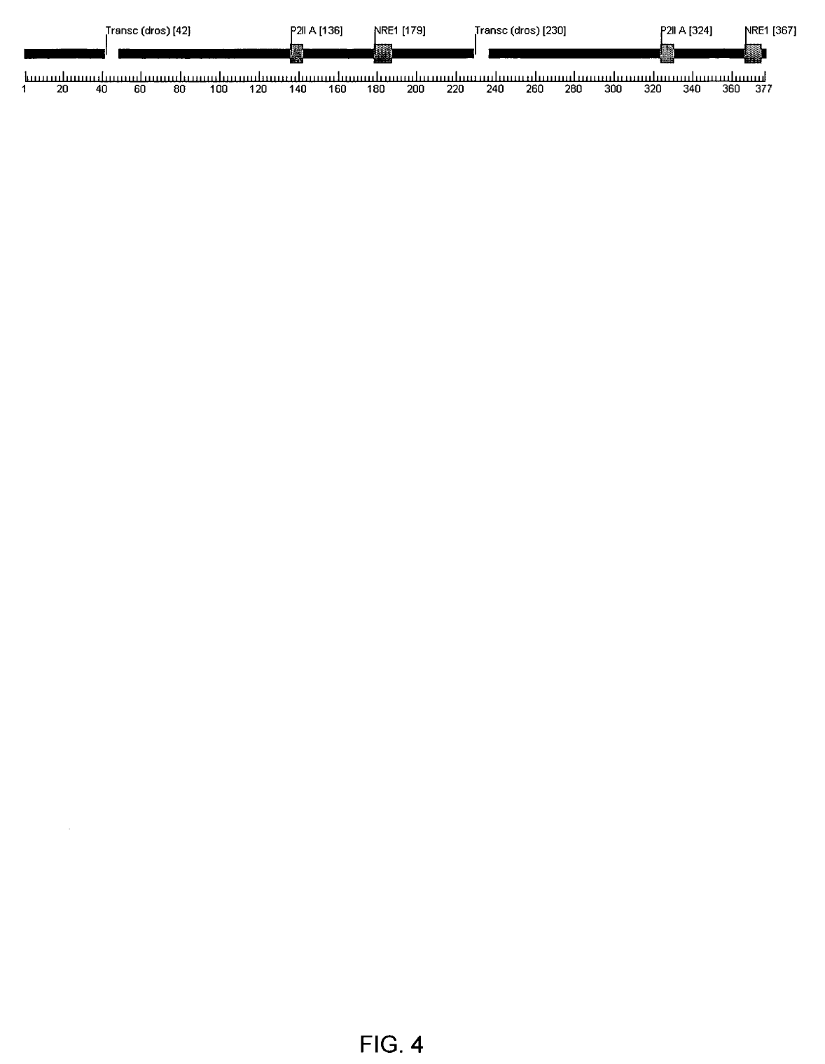

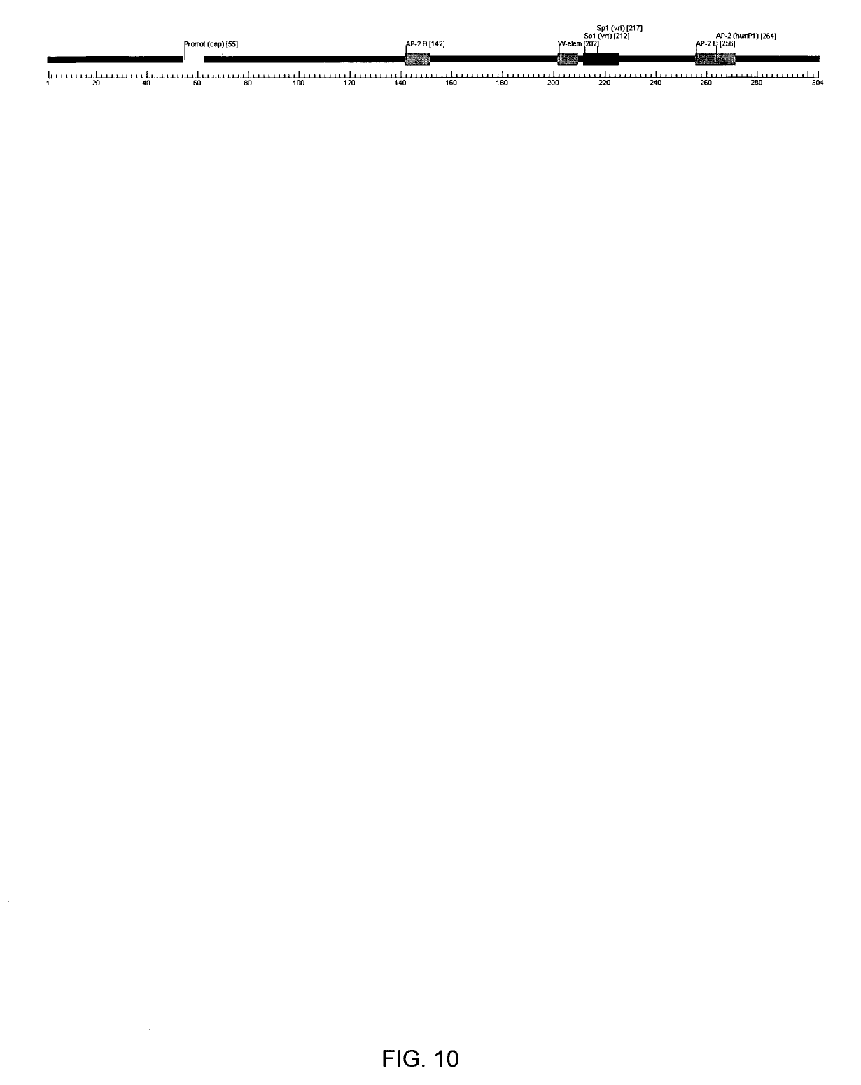

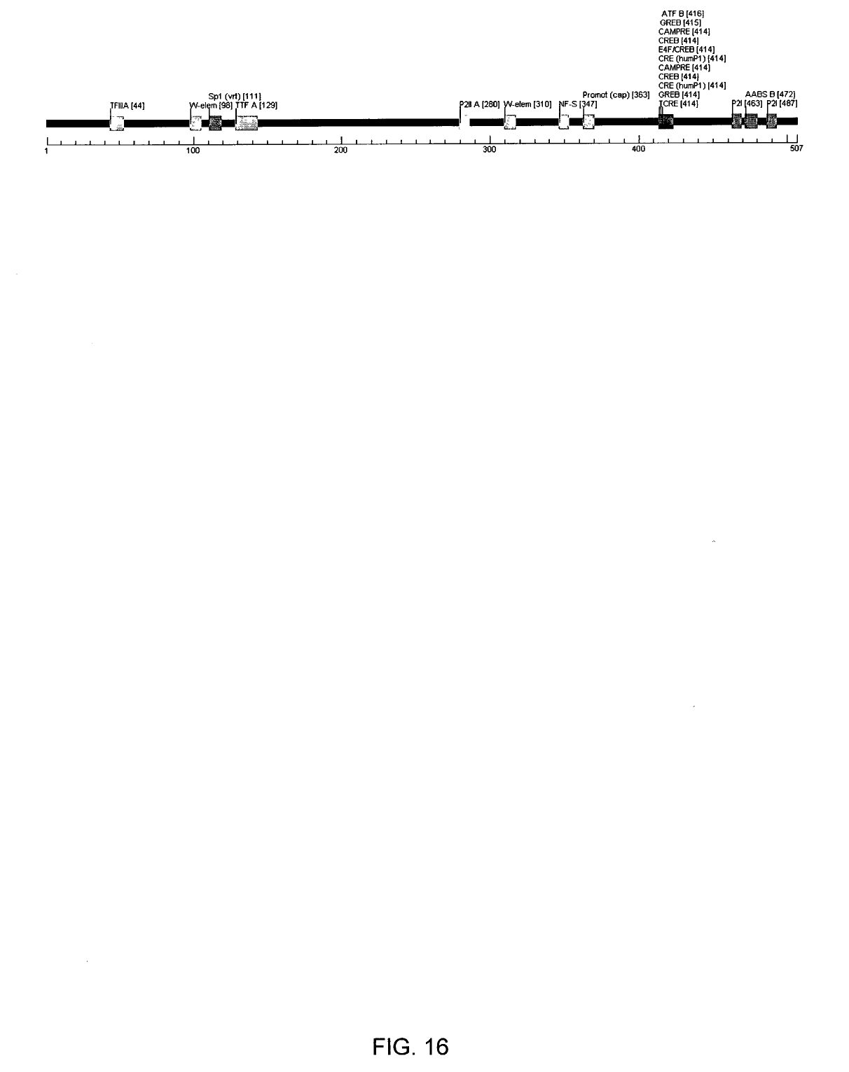

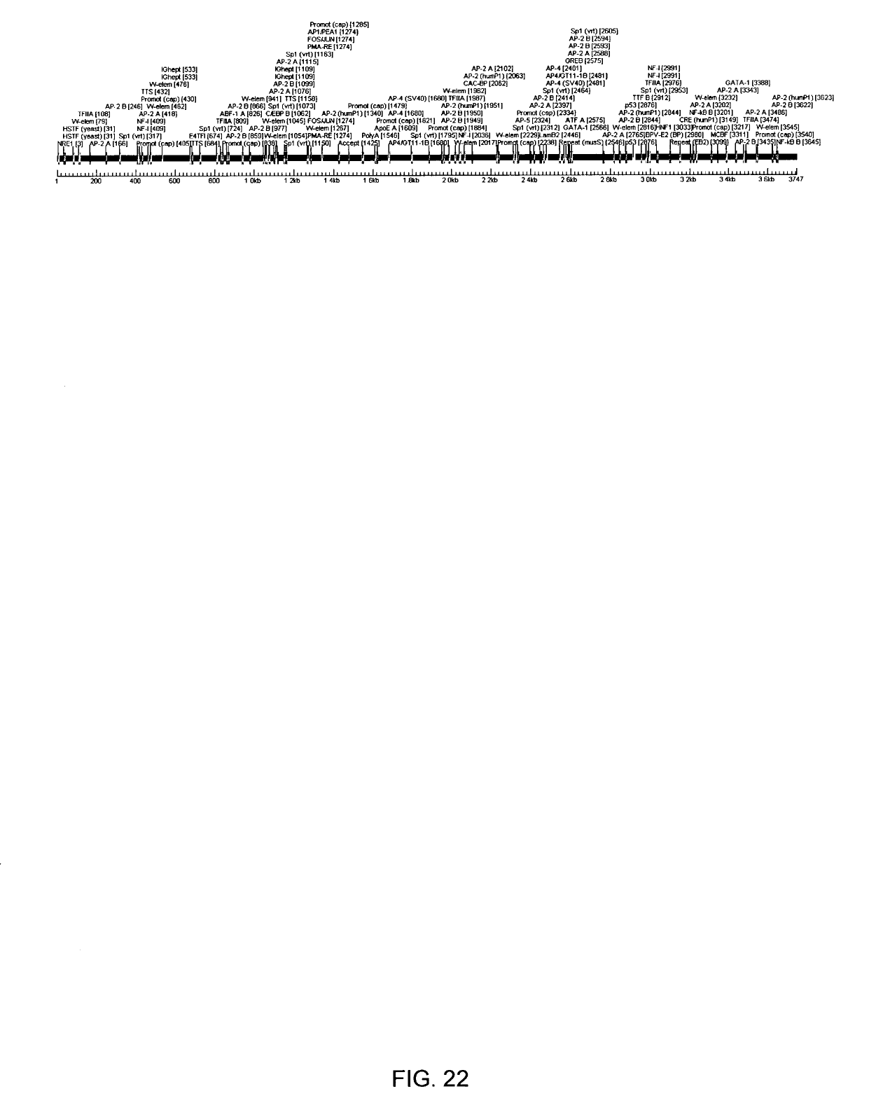

Computer analysis of the CNI-01054 sequence performed as described above revealed a number of transcriptional elements, e.g. binding sites for transcription factors (see FIG. 4). Thus, the CNI-01054 regulatory sequence likely responds to a variety of exogenous factors and environmental stimuli. Computer analysis of the CNI-01056 sequence performed as described above revealed a number of transcriptional elements, e.g. binding sites for transcription factors (see FIG. 10). Thus, the CNI-01056 regulatory sequence likely responds to a variety of exogenous factors and environmental stimuli. Computer analysis of the CNI-01058 sequence performed as described above revealed a number of transcriptional elements, e.g. binding sites for transcription factors (see FIG. 16). Thus, the CNI-01058 regulatory sequence likely responds to a variety of exogenous factors and environmental stimuli. Computer analysis of the CNI-01059 sequence performed as described above revealed a number of transcriptional elements, e.g. binding sites for transcription factors (see FIG. 22). Thus, the CNI-01059 regulatory sequence likely responds to a variety of exogenous factors and environmental stimuli.

The invention also provides regulatory sequences containing binding sites for various transcription factors. Thus, in one embodiment, the transcription modulating nucleic acid sequence contains at least 20 contiguous nucleotides of SEQ ID NO: 1 and at least one of the transcription factor binding sites of FIG. 4. IN another embodiment, the transcription modulating nucleic acid sequence contains at least 20 contiguous nucleotides of SEQ ID NO: 2, and at least one of the transcription factor binding sites of FIG. 10. In another embodiment, the transcription modulating nucleic acid sequence contains at least 20 contiguous nucleotides of SEQ ID NO: 3, and at least one of the transcription factor binding sites of FIG. 16. In another embodiment, the transcription modulating nucleic acid sequence contains at least 20 contiguous nucleotides of SEQ ID NO: 4, and at least one of the transcription factor binding sites of FIG. 22.

Regulatory sequences can also be physically mapped using restriction endonucleases to create restriction maps, which can easily be constructed. Such maps may be constructed by restricting the sequence with a variety of restriction enzymes, separating the resulting fragments on an agarose gel, and therefrom determining the relative positions of the restriction enzyme recognition sequences. Alternatively, since the recognition sequences of most restriction enzymes are well known to those of skill in the art, a restriction map may be generated once the nucleotide sequence of the promoter or regulatory sequence is determined.

Finer mapping of regulatory sequences can routinely be accomplished using site-directed mutagenesis, using variants of the fragments of the present invention. Site-specific mutagenesis is a technique useful in the preparation of mutant promoter regions useful in identifying important promoter elements. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the mismatch junction being traversed. Typically, a primer of about 17 to 25 nucleotides in length is preferred, with about 5 to 10 residues on both sides of the junction of the sequence being altered.

In general, the technique of site-specific mutagenesis is well known in the art as exemplified by publications (Adelman et al., DNA 2:183 (1983)). As will be appreciated, the technique typically employs a phage vector which exists in both a single stranded and double stranded form. Typical vectors useful in site-directed mutagenesis include vectors such as the M13 phage (Messing et al., Meth. Enzymol. 101:20 (1981)). These phage are readily commercially available and their use is generally well known to those skilled in the art. Double stranded plasmids are also routinely employed in site directed mutagenesis which eliminates the step of transferring the gene of interest from a plasmid to a phage.

In general, site-directed mutagenesis in accordance herewith is performed by first obtaining a single-stranded vector or melting apart the two strands of a double stranded vector which includes within any of the nucleic acid regulatory sequences of the invention. An oligonucleotide primer bearing the desired mutated sequence is prepared, generally synthetically, for example by the method of Crea et al. Proc. Natl. Acad. Sci. U.S.A. 75:5765-5769 (1978). Primer sequences are, of course, based on the nucleotide sequences of the regulatory sequences of the invention i.e., SEQ ID NO: 1, SEQ ID NO: 2, SEQ ID NO: 3, or SEQ ID NO: 4. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the nucleic acid regulatory sequences of the invention using site-directed mutagenesis is provided as a means of producing useful regulatory sequence variants and is not meant to be limiting, as there are other ways in which sequence variants of the regulatory sequences of the invention may be obtained, such as chemical mutagenesis. For example, recombinant vectors containing the desired regulatory sequence may be treated with mutagenic agents to obtain sequence variants (see, e.g., a method described by Eichenlaub et al., J. Bact. 138(2):559-566 (1979) for the mutagenesis of plasmid DNA using hydroxylamine).

The present invention also provides for fragments, i.e., subsequences, of the CNI-01054 (SEQ. ID. NO 1) regulatory sequence, which fragments need not be transcription activating. Such fragments can be used to detect the CNI-01054 (SEQ. ID. NO 1) sequence in a human cell sample, or in a cell sample derived from one of the animal sources listed above. Such fragments may be at least about 5, 10, 20, 30, 40, 50, 75, 100, 200, 300, or 350 nucleotides in length, or no more than about 20, 30, 40, 50, 75, 100, 200, 300, or 350 nucleotides in length.

The present invention also provides for fragments, i.e., subsequences, of the CNI-01056 (SEQ. ID. NO 2) regulatory sequence, which fragments need not be transcription activating. Such fragments can be used to detect the CNI-01056 (SEQ. ID. NO 2) sequence in a human cell sample, or in a cell sample derived from one of the animal sources listed above. Such fragments may be at least about 5, 10, 20, 30, 40, 50, 75, 100, 200, or 250 nucleotides in length, or no more than about 20, 30, 40, 50, 75, 100, 200, or 250 nucleotides in length.

The present invention also provides for fragments, i.e., subsequences, of the CNI-01058 (SEQ ID NO: 3) regulatory sequence, which fragments need not be transcription activating. Such fragments can be used to detect the CNI-01058 (SEQ ID NO: 3) sequence in a human cell sample, or in a cell sample derived from one of the animal sources listed above. Such fragments maybe at least about 5, 10, 20, 30, 40, 50, 75, 100, 200, 300, 400, or 500 nucleotides in length, or no more than about 20, 30, 40, 50, 75, 100, 200, 300, 400, or 500 nucleotides in length.

The present invention also provides for fragments, i.e., subsequences, of the CNI-01059 (SEQ ID NO: 4) regulatory sequence, which fragments need not be transcription activating. Such fragments can be used to detect the CNI-01059 (SEQ ID NO: 4) sequence in a human cell sample, or in a cell sample derived from one of the animal sources listed above. Such fragments maybe at least about 5, 10, 20, 30, 40, 50, 75, 100, 200, 300, 400, 500, 750, 1000, 2000, 3000, or 3500 nucleotides in length, or no more than about 20, 30, 40, 50, 75, 100, 200, 300, 400, 500, 750, 1000, 2000, 3000, or 3500 nucleotides in length.

The nucleic acid regulatory sequences of the invention can be generated using techniques well known to those of skill in the art. For example, the sequences may be generated from nucleic acids derived from natural sources or from publicly available cloned sequences by any one of a number of means known in the art, i.e., cleavage by one or more restriction endonucleases; DNaseI treatment; exonuclease treatment or mechanical shearing. Such fragments may also be constructed artificially. For example, fragments maybe synthesized chemically, or may be generated by means of the polymerase chain reaction (PCR).

The process of selecting and preparing a nucleic acid segment that includes a contiguous sequence from the genomic sequence region may alternatively be described as preparing a nucleic acid fragment. Of course, fragments may also be obtained by other techniques such as, e.g., by mechanical shearing, exonuclease treatment or by restriction enzyme digestion. Small nucleic acid segments or fragments may be readily prepared by, for example, directly synthesizing the fragment by chemical means, as is commonly practiced using an automated oligonucleotide synthesizer. Also, fragments may be obtained by application of nucleic acid reproduction technology, such as the PCR technology of U.S. Pat. No. 4,603,102, (incorporated herein by reference), by introducing selected sequences into recombinant vectors for recombinant production, and by other recombinant DNA techniques generally known to those of skill in the art of molecular biology.

The sequence of a particular regulatory sequence may be determined by a number of means well known in the art, including but not limited to the method of Maxam and Gilbert (Meth. Enzymol. 65:499-560 (1980)), the Sanger dideoxy method (Sanger, F., et al., Proc. Natl. Acad. Sci. U.S.A. 74:5463 (1977)), the use of T7 DNA polymerase (Tabor and Richardson, U.S. Pat. No. 4,795,699), or use of an automated DNA sequencer (e.g., Applied Biosystems, Foster City, Calif.). The labels used in sequencing may be radioactive or fluorescent.

The ability of any of the foregoing sequences to modulate, activate or enhance gene expression in a cell is straightforward. A vector suitable for maintenance and gene expression in a host cell is constructed, whereby the vector contains a reporter gene operably linked to the particular regulatory sequence or transcription activating sequence of the invention. The vector containing the regulatory sequence or transcription activating sequence is then placed in to a cell, preferably a neural cell or cell derived from the brain. After culturing for a period of time suitable for the reporter gene to express the reporter gene product, the amount of the reporter gene product is assessed. For example, if the reporter gene product is GFP, the amount of GFP is determined by assessing the amount of fluorescence emitted by the cell. A nucleotide sequence that modulates reporter gene expression according to the invention is one that causes a detectable difference of the level of expression of the reporter gene, and/or amount of the reporter gene product, when compared to a control cell containing the vector and reporter gene, but lacking the regulatory sequence or transcriptional activating sequence. In a preferred embodiment, the difference is an increase in the expression of the reporter gene over that of the control.

5.2. Vectors and Regulation of Gene Expression

The present invention provides the CNI-01054, CNI-01056, CNI-01058 and CNI-01059.regulatory sequences, or transcription modulating sequences thereof contained in a vector. The regulatory sequences of the present invention each promotes or enhance gene expression in cells derived from the nervous system; thus, each of these regulatory sequences or nucleic acid regulatory sequences thereof are useful for the expression of a coding sequence in cells, particularly in nervous system cells.

The invention further provides vectors comprising a nucleic acid regulatory molecule of the invention. In this regard, in one embodiment, the invention provides a vector comprising the nucleotide sequence of SEQ ID NO: 1, SEQ ID NO: 2, SEQ ID NO: 3 or SEQ ID NO: 4. Additionally, the invention further provides vectors comprising two or more of the nucleotide sequences of these SEQ ID NOs.

In another embodiment, the vector comprises the nucleotide sequence of a transcription activating sequence of SEQ ID NO: 1 or the reverse complement of SEQ ID NO: 1. For example, the transcription activating sequence of SEQ ID NO: 1 may be at least about 20, 30, 40, 50, 75, 100, 200, 300, or 350 nucleotides in length. The vector may also include a nucleic acid that hybridizes along its entire length to SEQ ID NO: 1, or SEQ ID NO: 2, or SEQ ID NO: 3, or SEQ ID NO: 4. The transcription activating sequence of SEQ ID NO: 2 may be at least about 20, 30,40, 50,75, 100,200 or 250 nucleotides in length. The transcription activating sequence of SEQ ID NO: 3 may be at least about 20, 30, 40, 50, 75, 100, 200, 300, 400 or 500 nucleotides in length. The transcription activating sequence of SEQ ID NO: 4 may be at least about 20, 30, 40, 50, 75, 100, 200, 300, 400, 500, 750, 1000, 2000, 3000 or 3500 nucleotides in length.

In another embodiment, the vector further comprises a coding sequence operably linked to a nucleic acid regulatory sequence of the invention. In a more specific embodiment, the coding sequence is heterologous to the nucleic acid regulatory sequence of the invention. For example, the coding sequence can encode a peptide or a polypeptide and can comprise, e.g., a reporter gene sequence or a neuroprotective sequence.

With respect to a reporter gene sequence, such a sequence can encode, for example, β-galactosidase, a fluorescent protein (e.g., a green, red, blue, or cyan fluorescent protein), chloramphenicol acetyltransferase, luciferase or an antigenic marker.

In another embodiment, the vector further comprises a multiple cloning site (MCS), wherein when a nucleotide sequence is inserted into the MCS, the nucleotide sequence is operably linked to a nucleic acid regulatory sequence of the invention. In yet another embodiment, a vector of the invention can further comprise a coding sequence within the MCS. In yet another embodiment, a vector of the invention can further comprise an internal ribosomal entry site (IRES). The invention further provides that any vector of the invention can also contain regulatory sequence (e.g., promoter sequence) in addition to the nucleic acid regulatory sequence of the invention.

The invention also provides for the enhancement of expression of a nucleotide sequence of interest in a vector containing the nucleotide sequence operably linked to a promoter sequence heterologous to the nucleic acid molecule of the invention. In this regard, in one embodiment, the invention provides a vector comprising a nucleic acid regulatory sequence of the invention, a promoter, and an MCS operably linked in an upstream-to-downstream order, such that when the nucleotide sequence of interest is present within the MCS, expression of the nucleotide sequence of interest is enhanced relative to its expression from the vector in the absence of the nucleic acid regulatory sequence of the invention. In one embodiment, the vector further comprises an IRES. In another embodiment, the vector further comprises a coding sequence within the MCS. In a more specific embodiment, the coding sequence is heterologous to the nucleic acid regulatory sequence of the invention. For example, the coding sequence can encode a peptide or a polypeptide and can comprise, e.g., a reporter gene sequence or a neuroprotective sequence.

With respect to a reporter gene sequence, such a sequence can encode, for example, β-galactosidase, a fluorescent protein (e.g., a green, red, blue, or cyan fluorescent protein), chloramphenicol acetyltransferase, luciferase or an antigenic marker.

Any of the vectors of the invention can be adapted for transfer to a eukaryotic host cell, including a human host cell. In a more specific embodiment, the eukaryotic host cell is a nervous system cell. In a more specific embodiment, the nervous system cell is a nervous system cell line, glial cell, astrocyte, oligodendrocyte, mesencephalic neuron, hypothalamic neuron or cortical neuron. In another embodiment, the vectors above are adapted for transfer to a prokaryotic host cell.

A wide variety of heterologous gene sequences can be expressed under the control of the nucleic acid regulatory sequences of the invention. Such gene sequences include, but are not limited to, sequences encoding neuroprotective sequences, reporter gene products, toxic gene products, potentially toxic gene products, antiproliferation or cytostatic gene products. Reporter genes can also be expressed including enzymes, (e.g. Chloramphenicol Acetyl Transferase (CAT), beta-galactosidase, luciferase, light-emitting proteins such as those encoded by luxAB, fluorescent proteins such as a green, red, blue, or cyan fluorescent protein, or antigenic markers.

A person of skill in the art would understand that the nucleic acid regulatory sequences of the invention can be used to modulate the expression of a gene contained in an expression vector that either possesses or lacks a promoter. Such an expression vector typically possesses a multiple cloning site upstream of the start codon of a gene. The vector may or may not possess a promoter between the MCS and the gene. Where the plasmid lacks a promoter, an increase in the expression of the gene indicates that the cloned genomic fragment has promoter activity, or promoter and enhancer activities. Where the plasmid possesses a promoter, an increase in the expression of the gene indicates that the cloned fragment possesses at least enhancer activity. It will be apparent to one of skill in the art that the genomic fragment may be cloned in either orientation, the method of generating the fragment permitting. For example, genomic fragments generated by DNase I treatment, shearing, or restriction with a single restriction endonuclease may be inserted in either orientation. Fragments generated by filling-in and/or digestion with a single-strand nuclease, thereby generating blunt-ended fragments, can be inserted in either orientation. Alternatively, directional cloning can be achieved by restriction with a pair of restriction endonucleases, each having a different recognition sequence.

The genomic fragment representing a regulatory sequence may be inserted in multiple copies upstream of a gene to be expressed, perhaps improving the regulatory activities. Furthermore, the regulatory sequence or fragment thereof need not be placed in an adjacent conformation and may be separated by numerous random nucleotides and still retain their improved regulatory and promotion capability.

The regulatory sequences and transcription activating fragments thereof of the present invention may be used to induce expression of a heterologous gene in cells derived from the nervous system, such as neurons, including cortical neurons, hippocampal neurons, mesencephalic neurons, medullary neurons, and glial cells. The invention further provides for host cells, or progeny thereof, containing the vectors above. In a more specific embodiment, said host cell is a eukaryotic cell, including a human host cell. In a more specific embodiment, said host cell is a nervous system cell. In another specific embodiment, said host cell is a prokaryotic cell. In cases where such cells are tumor cells, the induction of a cytotoxic product by the the regulatory sequences of the present invention may be used as a form of cancer gene therapy. Additionally, antisense, antigene, or aptameric oligonucleotides may be delivered to cells using the presently described expression constructs. Ribozymes or single-stranded RNA can also be expressed in a cell to inhibit the expression of a particular gene of interest. The target genes for these antisense or ribozyme molecules should be those encoding gene products that are essential for cell maintenance.

5.3. Genetically Engineered Host Cells

The regulatory sequences disclosed herein may be inserted into a variety of expression vectors for introduction into host cells. Thus, the invention further provides for host cells, or progeny thereof, containing the vectors above. In a more specific embodiment, said host cell is a eukaryotic cell, including a human host cell. In a more specific embodiment, said host cell is a nervous system cell. In another specific embodiment, said host cell is a prokaryotic cell. In this context, “host cells” means both cells, generally prokaryotic, used to maintain genetic constructs comprising the regulatory sequences of the present invention and a gene of interest that this region controls, as well as cells, generally eukaryotic, in which expression of the gene of interest is desired. In a preferred embodiment, the expression vector or the nucleic acid regulatory sequence of the invention is engineered to be stably integrated into the eukaryotic host cell genome.

The invention further provides a method of expressing a coding sequence in a host cell in cell culture. In one embodiment, the method comprises culturing a host cell containing a vector of the invention that contains a coding sequence under conditions effective to allow expression of the coding sequence by said host cell. In another embodiment, the method comprises culturing a host cell of the invention wherein the nucleic acid regulatory sequence controls expression of a coding sequence endogenously present in the genome of said host cell, under conditions effective to allow expression of the coding sequence by said host cell.

The invention also provides a method of producing a peptide or polypeptide comprising maintaining a host cell of the invention that contains a coding sequence that encodes a peptide or polypeptide under conditions effective to allow expression of said coding sequence, and to allow translation of the resulting mRNA, such that a peptide or polypeptide is expressed. In one embodiment, the coding sequence is present as part of a vector of the invention. In another embodiment, the host cell has been engineered such that a nucleic acid regulatory sequence of the invention controls expression of a coding sequence endogenously present in the genome of said host cell, under conditions effective to allow expression of the coding sequence by said host cell. In a more specific embodiment, the vector is present in the genome of said host cell.

In bacterial systems a number of expression vectors may be advantageously selected depending upon the use intended for the expressed product; the promoter or regulatory sequences contained therein can be replaced by one or more of the regulatory sequences of the present invention, i.e., SEQ ID NO: 1, SEQ ID NO: 2, SEQ ID NO: 3, SEQ ID NO: 4, or transcription regulating sequences thereof. Such vectors include, but are not limited to, the E. coli expression vector pUR278 (Ruther et al., EMBO J. 2:1791 (1983)), in which a coding sequence may be ligated into the vector in frame with the lacZ coding region so that a hybrid protein is produced; pIN vectors (Inouye & Inouye, Nucleic Acids Res. 13:3101-3109 (1985); Van Heeke & Schuster, J. Biol. Chem. 264:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned polypeptide of interest can be released from the GST moiety.

In yeast, a number of vectors containing constitutive or inducible promoters can be replaced by the regulatory sequence of the invention and fragments thereof (CURRENT PROTOCOLS IN MOLECULAR BIOLOGY, Vol. 2, Ed. Ausubel et al., Greene Publish. Assoc. & Wiley Interscience, Ch. 13 (1988); Grant et al., Expression and Secretion Vectors for Yeast, in METHODS IN ENZYMOLOGY, Eds. Wu & Grossman, Acad. Press, N.Y., Vol. 153, pp. 516-544 (1987); Glover, DNA CLONING, Vol. 11, IRL Press, Wash., D.C., Ch. 3 (1986); and Bitter, Heterologous Gene Expression in Yeast, METHODS IN ENZYMOLOGY, Eds. Berger & Kimmel, Acad. Press, N.Y., Vol. 152, pp. 673-684 (1987); and THE MOLECULAR BIOLOGY OF THE YEAST SACCHAROMYCES, Eds. Strathem et al., Cold Spring Harbor Press, Vols. I and II (1982)).

In mammalian host cells, a number of commercially available vectors can be engineered to insert the regulatory sequence of the invention (Clontech, Palo Alto, Calif.).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used.

For expression in nervous system-specific host cells, the host cells may be derived from the nervous system itself, and grown in culture, or may be established neuronal or neuron-like cell lines. In reference to neuronal cell lines, many neuronal clones exist which have been used extensively as model systems of development since they retain electrophysiological activity with appropriate surface receptors, specific neurotransmitters, synapse forming properties and the ability to differentiate morphologically and biochemically into normal neurons. Such cells are described in the following references: Kimhi et al., Proc. Natl. Acad. Sci. USA 73:462-466 (1976); In: EXCITABLE CELLS IN TISSUE CULTURE, Nelson, P. G. et al., eds., Plenum Press, New York, pp. 173-245 (1977); Prasad, K. M. et al., In: CONTROL OF PROLIFERATION OF ANIMAL CELLS, Clarkson, B. et al., eds., Cold Spring Harbor Laboratory Press, Cold Spring Harbor, N.Y., pp. 581-594 (1974); Puro et al., Proc. Natl. Acad. Sci. USA 73:3544-3548 (1976); Notter et al., Devel. Brain Res. 26:59-68 (1986); Schubert et al., Proc. Natl. Acad. Sci. USA 67:247-254 (1970); Kaplan et al., In: BASIC AND CLINICAL ASPECTS OF MOLECULAR NEUROBIOLOGY, Guffrida-Stella, A. M. et al., eds., Milano Fondozione International Manarini (1982)) (see also U.S. Pat. No. 6,020,197 (describing methods of culturing neuroblasts).

The expression vectors that contain the nucleic acid regulatory sequences of the invention may contain a gene encoding a selectable marker. A number of selection systems may be used, including but not limited to, the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:2026 (1962)), and adenine phosphoribosyltransferase (Lowy et al., Cell 22:817 (1980)) genes can be employed in tk⁻, hgprt⁻ or aprt⁻ cells, respectively. Also, antimetabolite resistance can be used as the basis of selection for dhfr, which confers resistance to methotrexate (Wigler et al., Proc. Natl. Acad. Sci. USA 77:3567 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Colberre-Garapin et al., J. Mol. Biol. 150:1 (1981)); and hygro, which confers resistance to hygromycin (Santerre, et al., Gene 30:147 (1984)) genes. Additional selectable genes include trpB, which allows cells to utilize indole in place of tryptophan; hisD, which allows cells to utilize histinol in place of histidine (Hartman & Mulligan, Proc. Natl. Acad. Sci. USA 85:8047 (1988)); ODC (omithine decarboxylase) which confers resistance to the omithine decarboxylase inhibitor, 2-(difluoromethyl)-DL-omithine, DFMO (McConlogue, In: CURRENT COMMUNICATIONS IN MOLECULAR BIOLOGY, 1987, Cold Spring Harbor Laboratory ed.) and glutamine synthetase (Bebbington et al., Biotech 10:169 (1992)).

Introduction of the nucleic acid, comprising the nucleic acid regulatory sequence and, optionally, the coding sequence to be expressed, into the cell is accomplished by such methods as electroporation, lipofection, calcium phosphate mediated transfection, viral infection, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217: 599-618 (1993); Cohen et al., Meth. Enzymol. 217: 618-644 (1993); Cline, Pharmac. Ther. 29: 69-92 (1985)) and may be used in accordance with the present invention, provided that the necessary developmental and physiological functions of the recipient cells are not disrupted. The chosen technique preferably provides for the stable transfer of the nucleic acid to the cell, so that the nucleic acid is expressible by the cell and is heritable and expressible by its cell progeny.

5.4. Screening for Modulators

The invention also provides a method of identifying a modulator of a nucleic acid regulatory sequence of the invention, comprising: (a) contacting a host cell containing a nucleic acid regulatory sequence of the invention operably linked to a reporter gene sequence with a test compound; and (b) assaying expression of the reporter gene, such that, if a change in reporter gene expression relative to its expression in the absence of the test compound, is detected, a modulator of the nucleic acid regulatory sequence is identified. In a particular embodiment, the host cell is a nervous system cell.

In a specific embodiment of the invention, the genetically-engineered cell lines of Section 5.3., supra may be used to screen for peptides, polypeptides, small molecules, natural and synthetic compounds or other cell bound or soluble molecules that cause a stimulation or inhibition of transcriptional activities of the regulatory sequences of the invention. Such compounds may, for example, be used to control gene expression in cells in vitro that is mediated by a regulatory sequence of the present invention.

Random peptide libraries consisting of all possible combinations of amino acids attached to a solid phase support may be used to identify peptides that are able to activate or inhibit the activities of the regulatory sequences of the invention (Lam et al., Nature 354: 82-84 (1991)). The screening of peptide libraries may have therapeutic value in the discovery of pharmaceutical agents that stimulate or inhibit gene expression of mediated or controlled by one or more of the regulatory sequences of the invention. In addition, combinatorial chemistry libraries can also be screened.

An example of an in vitro screening assay is described below. About 10,000 cells per well are plated in 96-well plates in total volume of 100 μl, using medium appropriate for each cell line. A reporter plasmid is used or constructed whereby the expression of a gene for luciferese is placed under the control of one or more of the regulatory sequences of the invention. In the following day, this reporter plasmid is transfected into the cells, using 50 ng plasmid per well in the presence of LipofectAmine cationic lipid transfection reagent (Gibco) at 16 μg/ml. Final volume of the transfection mix is 100 μl. Potential inhibitors of gene expression controlled by one or more of the regulatory sequences of the invention can also be added to the cells at this time. The effect of the such inhibitors can be determined by measuring the response of the luciferase reporter gene driven by the regulatory sequence(s). After 6 hr. incubation, 100 μg DMEM medium+2.5% fetal bovine serum (FBS) to 1.25% final serum concentration is added to the cells, and incubated a total of 24 hr (18 hr more). At 24 hr, the plates are washed with PBS, blot dried, and frozen at −80° C. The plates are thawed the next day and 200 μg luciferin (LucLite, Packard) reagent is added to each well. The plates are counted in TopCount scintillation counter to determine RLU (relative luciferase units). In the above assay, the reporter can also be a fluorescent protein such as green fluorescent protein (GFP). This assay can easily be set up in a high-throughput screening mode for evaluation of compound libraries in a 96-well format.

5.5. Modification of Gene Expression

5.5.1. Modification of Regulatory Sequence-Controlled Gene Expression

Under certain circumstances, it is desirable to modify the expression of a gene controlled in cis by the regulatory sequences of the invention. This modification can constitute increasing the activity of the regulatory sequences, or inhibiting their activity. Thus, the invention provides means for promoting or increasing the activity of the regulatory sequences, and thereby increasing or promoting the expression of a gene or genes controlled by one or more sequences of the invention. The invention further provides for inhibiting the regulatory activity of the regulatory sequences, and thereby inhibiting the expression of a gene or genes controlled by one or more sequences of the invention.

The endogenous counterparts of the regulatory sequences of the invention may be targeted to specifically down regulate expression of the genes under their control. For example, oligonucleotides complementary to the regulatory sequences may be designed and delivered to cells that contain a gene under the control of the a regulatory sequence of the present invention. Such oligonucleotides anneal to the regulatory sequence, and prevent activation of transcription. Alternatively, the regulatory sequence or portions thereof may be delivered to cells in saturating concentrations to compete for transcription factor binding. For general reviews of the methods of gene therapy, see Goldspiel et al., Clinical Pharmacy 12:488-505 (1993); Wu and Wu, Biotherapy 3:87-95 91991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); also TIBTECH 11(5):155-215 (1993). Methods commonly known in the art of recombinant DNA technology that can be used are described in Ausubel et al. (eds.), 1993, CURRENT PROTOCOLS IN MOLECULAR BIOLOGY, John Wiley & Sons, NY; and Kriegler, 1990, GENE TRANSFER AND EXPRESSION, A LABORATORY MANUAL, Stockton Press, NY.

In a specific embodiment, the nucleic acid is directly administered in vivo into a target cell. This can be accomplished by any methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), by direct injection of naked DNA, by use of microparticle bombardment (e.g., a gene gun; Biolistic, Dupont), by coating with lipids or cell-surface receptors or transfecting agents, by encapsulation in liposomes, microparticles, or microcapsules, by administering it in linkage to a peptide known to enter the nucleus, or by administering it in linkage to a ligand subject to receptor-mediated endocytosis (see, e.g., Wu and Wu, 1987, J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180, published Apr. 16, 1992; WO 92/22635, published Dec. 23, 1992; WO92/20316, published Nov. 26, 1992; WO93/14188, published Jul. 22, 1993; WO 93/20221, published Oct. 14, 1993). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

The oligonucleotide may comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Endogenous target gene expression can also be reduced by inactivating or “knocking out” a regulatory sequence using targeted homologous recombination (e.g., see Smithies, et al., Nature 317:230-234 (1985); Thomas and Capecchi, Cell 51:503-512 (1987); Thompson et al., Cell 5:313-321 (1989); each of which is incorporated by reference herein in its entirety). For example, a non-functional target sequence (or a completely unrelated DNA sequence) flanked by DNA homologous to the specific regulatory sequence can be used, with or without a selectable marker and/or a negative selectable marker, to transfect cells that express the target gene in vivo. Insertion of the DNA construct, via targeted homologous recombination, results in inactivation of the specific regulatory sequence (Chappel, 1993, U.S. Pat. No. 5,272,071). This approach can be adapted for use in humans provided the recombinant DNA constructs are directly administered or targeted to the required site in vivo using appropriate vectors.

Alternatively, endogenous target gene expression can be reduced by targeting deoxyribonucleotide sequences complementary to the regulatory sequence of the target gene (i.e., the target gene promoter and/or enhancers) to form triple helical structures that prevent transcription of the target gene in target cells in the body (see generally, Helene, Anticancer Drug Des., 6(6):569-584 (1991); Helene et al., Ann. N.Y. Acad. Sci., 660:27-36 (1992); and Maher, Bioassays 14(12):807-815 (1992)).

Nucleic acid molecules to be used in triple helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

The anti-sense RNA and DNA molecules and triple helix molecules of the invention may be prepared by any method known in the art for the synthesis of nucleic acid molecules. These include techniques for chemically synthesizing oligodeoxyribonucleotides well known in the art such as for example solid phase phosphoramidite chemical synthesis. Alternatively, RNA molecules may be generated by in vitro and in vivo transcription of DNA sequences encoding the RNA molecule. Alternatively, antisense cDNA constructs that synthesize antisense RNA constitutively or inducibly, depending on the promoter used, can be introduced stably into cell lines.

Various modifications to the DNA molecules may be introduced as a means of increasing intracellular stability and half-life. Possible modifications include, but are not limited to, the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

5.5.2. Modification of Expression of Non-cis-Linked Genes Using Regulatory Sequences of the Invention

The expression of genes not operably linked to one of the disclosed regulatory sequences can be accomplished by use of antisense nucleic acids. In this regard, the regulatory sequences promote or enhance the expression of a nucleotide sequence that has exact or substantial complementarity to a gene whose expression is to be down regulated. Alternatively, downregulation of non-cis-linked genes by a regulatory sequence of the invention may be accomplished by using the regulatory sequence to drive the production of mRNA that folds into a ribozyme, which is able to cleave the mRNA produced by the gene whose downregulation is sought. Antisense RNA and DNA molecules act to directly block the translation of mRNA by hybridizing to targeted mRNA and preventing protein translation. Antisense approaches involve the design of oligonucleotides which are complementary to a protective sequence mRNA. The antisense oligonucleotides will bind to the complementary sequence in mRNA transcripts and prevent translation. Absolute complementarity, although preferred, is not required.

A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In one embodiment, oligonucleotides complementary to non-coding regions of a gene to be downregulated could be used in an antisense approach to inhibit translation of endogenous mRNA. Antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit protective sequence expression. It is preferred that these studies utilize controls that distinguish between antisense gene inhibition and nonspecific biological effects of oligonucleotides. It is also preferred that these studies compare levels of the cerebral RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleic acid of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger, et al., Proc. Natl. Acad. Sci. U.S.A. 86:6553-6556 (1988); Lemaitre, et al., Proc. Natl. Acad. Sci. U.S.A. 84:648-652 (1987); U.S. Pat. No. 4,904,582) or the blood-brain barrier (see, e.g., PCT Publication No. WO89/10134, published Apr. 25, 1988), hybridization-triggered cleavage agents (see, e.g., Krol et al., BioTechniques 6:958-976 (1988)) or intercalating agents (see, e.g., Zon, Pharm. Res. 5:539-549 (1988)). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier, et al, Nucl. Acids Res. 15:6625-6641 (1987)). The oligonucleotide is a 2′-O-methylribonucleotide (Inoue, et al., Nucl. Acids Res. 15:6131-6148 (1987)), or a chimeric RNA-DNA analogue (Inoue, et al., FEBS Lett. 215:327-330 (1987)).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein, et al. (Nucl. Acids Res. 16:3209 (1988)), methylphosphonate oligonucleotides can be prepared by use of controlled pore glass polymer supports (Sarin, et al., Proc. Natl. Acad. Sci. U.S.A. 85:7448-7451 (1988)), etc.

While antisense nucleotides complementary to the coding region sequence of the gene to be downregulated are useful, antisense nucleotides complementary to the transcribed, untranslated region are most preferred.

Antisense molecules should be delivered to cells that express the gene to be down regulated in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies which specifically bind receptors or antigens expressed on the target cell surface) can be administered systemically.

A preferred approach to achieve intracellular concentrations of the antisense sufficient to suppress translation of endogenous mRNAs utilizes a recombinant DNA construct in which the antisense oligonucleotide is placed under the control of a strong promoter. The use of such a construct to transfect target cells in a patient will result in the transcription of sufficient amounts of single stranded RNAs which will form complementary base pairs with the endogenous protective sequence transcripts and thereby prevent translation of the protective sequence mRNA. For example, a vector can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Any type of plasmid, cosmid, YAC or viral vector can be used to prepare the recombinant DNA construct that can be introduced directly into the tissue site. Alternatively, viral vectors can be used that selectively infect the desired tissue, in which case administration may be accomplished by another route (e.g., systemically).

Ribozyme molecules designed to catalytically cleave target gene mRNA transcripts can also be used to prevent translation of target gene mRNA and, therefore, expression of target gene product (see, e.g., PCT International Publication WO90/11364, published Oct. 4, 1990; Sarver et al., Science 247, 1222-1225(1990)).

Ribozymes are enzymatic RNA molecules capable of catalyzing the specific cleavage of RNA (for a review, see Rossi, Current Biology 4:469-471(1990)). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

While ribozymes that cleave mRNA at site-specific recognition sequences can be used to destroy target gene mRNAs, the use of hammerhead ribozymes is preferred. Hammerhead ribozymes cleave mRNAs at locations dictated by flanking regions which form complementary base pairs with the target mRNA. The sole requirement is that the target mRNA have the following sequence of two bases: 5′-UG-3′. The construction and production of hammerhead ribozymes is well known in the art and is described more fully in Myers, MOLECULAR BIOLOGY AND BIOTECHNOLOGY: A COMPREHENSIVE DESK REFERENCE, VCH Publishers, New York (1995) (see especially FIG. 4, page 833) and in Haseloff and Gerlach, Nature, 334:585-591 (1988), which is incorporated herein by reference in its entirety.

Preferably the ribozyme is engineered so that the cleavage recognition site is located near the 5′ end of the target gene mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., Science, 224:574-578 (1984); Zaug & Cech, Science, 231:470-475 (1986); Zaug, et al, Nature, 324:429-433 (1986); U.S. Pat. No. 4,987,071; Been & Cech, Cell, 47:207-216 (1986)). The Cech-type ribozymes have an eight nucleotide active site that hybridizes to a target RNA sequence cleavage of the target RNA takes place. The invention encompasses those Cech-type ribozymes that target eight nucleotide active site sequences that are present in the target gene.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive promoter, so that transfected cells will produce sufficient quantities of the ribozyme to destroy endogenous target gene messages and inhibit translation. Because ribozymes, unlike antisense molecules, are catalytic, a lower intracellular concentration is required for efficiency.

5.6. Transgenic Animals

The nucleic acid regulatory sequences of the invention can be used to direct expression of a coding sequence in animals by transgenic technology. Animals of any species, including, but not limited to, mice, rats, rabbits, guinea pigs, pigs, micro-pigs, goats, sheep, and non-human primates, e.g., baboons, monkeys, and chimpanzees may be used to generate transgenic animals. The term “transgenic,” as used herein, refers to animals expressing coding sequences from a different species (e.g., mice expressing human gene sequences), as well as animals that have been genetically engineered to overexpress endogenous (i.e., same species) sequences or animals that have been genetically engineered to no longer express endogenous gene sequences (i.e., “knock-out” animals), and their progeny.

Any technique known in the art may be used to introduce a transgene under the control of a regulatory sequence of the invention into animals to produce the founder lines of transgenic animals. Such techniques include, but are not limited to, pronuclear microinjection (Hoppe and Wagner U.S. Pat. No. 4,873,191); retrovirus-mediated gene transfer into germ lines (Van der Putten, et al., Proc. Natl. Acad. Sci., USA 82:6148-6152 (1985)); gene targeting in embryonic stem cells (Thompson, et al., Cell 56:313-321 (1989)); electroporation of embryos (Lo, Mol. Cell. Biol. 3:1803-1814 (1983)); and sperm-mediated gene transfer (Lavitrano et al., Cell 57:717-723 (1989)) (see also Gordon, Transgenic Animals, Intl. Rev. Cytol. 115, 171-229 (1989)).

Any technique known in the art may be used to produce transgenic animal clones containing a transgene, for example, nuclear transfer into enucleated oocytes of nuclei from cultured embryonic, fetal or adult cells induced to quiescence (Campbell, et al., Nature 380:64-66 (1996); Wilmut, et al., Nature 385:810-813 (1997)).

The present invention provides for transgenic animals that carry a transgene such as a reporter gene under the control of a regulatory sequence of the invention or transcription modulating sequences thereof in all their cells, as well as animals that carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (Proc. Natl. Acad. Sci. U.S.A 89:6232-6236 (1992)). In one embodiment, the expression characteristics of an endogenous gene within a cell, cell line or microorganism may be modified by inserting a regulatory sequence of the invention or transcription modulating sequence thereof, into the genome of a cell, stable cell line or cloned microorganism, by nonhomologous recombination, such that the inserted regulatory element is operatively linked with the endogenous gene and controls, modulates or activates the endogenous gene. For example, endogenous genes that are normally “transcriptionally silent,” i.e., one that is normally not expressed, or are expressed only at very low levels in a cell line or microorganism, may be activated by inserting a regulatory sequence of the invention, or transcription activating sequence thereof which is capable of promoting the expression of a normally expressed gene product in that cell line or microorganism.

A heterologous regulatory element may be inserted into a stable cell line or cloned microorganism, such that it is operatively linked with and activates expression of endogenous genes, using techniques, such as targeted homologous recombination, which are well known to those of skill in the art, and described e.g., in Chappel, U.S. Pat. No. 5,272,071; PCT publication No. WO 91/06667, published May 16, 1991; Skoultchi U.S. Pat. No. 5,981,214; Treco et al., U.S. Pat. No. 5,968,502 and PCT publication No. WO 94/12650, published Jun. 9, 1994. Alternatively, non-targeted (e.g., non-homologous) recombination techniques which are well known to those of skill in the art and described, e.g., in PCT publication No. WO 99/15650, published Apr. 1, 1999, may be used.

Once transgenic animals have been generated, the transcriptional activities of the specific regulatory sequence may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques that include, but are not limited to, northern blot analysis of tissue samples obtained from the animal, in situ hybridization analysis, and RT-PCR. Samples of transgene-expressing tissue, may also be evaluated immunocytochemically using antibodies specific for the transgene product. Such animals may be used as in vivo system for the screening of agents that activate or inhibit the activities of the regulatory sequence.

5.7. Therapeutics and Diagnostics

5.7.1. Therapeutic Uses of Regulatory Sequences

DNA sequences that regulate cell-, tissue- or organ-specific transcription may be used therapeutically or prophylactically. Such sequences can be inserted into DNA vector and used to control cell-, tissue-, region- or nervous system-specific transcription of an introduced gene or DNA sequence, or an antisense form of a gene, in order to alter the expression of endogenous cellular genes, or to cause expression of factors (e.g., secreted cytokines) that will alter the properties of other cells. For example, it may be possible to use neuron-specific regulatory sequences to express the antisense forms of factors responsible for the excess process outgrowth in neurons that is associated with epilepsy. Alternatively, categories of genes associated with nerve regeneration can be placed under control of inducible promoters associated with regions of DNA that regulate neuron-specific expression. Other applications may be the prophylactic or therapeutic expression of factors that would confer resistance to the effects of chronic infectious agents such as viruses or bacteria that harm cells in the CNS. For example, synthetic antisense molecules (e.g., phosphorothioate oligodeoxynucleotides) are known to suppress HIV infection in vitro, but toxicity has prevented these compounds from progressing through clinical trials. Since HIV infection can affect the CNS, it may be possible to replace damaged nervous system tissue with nervous system stem cells stably expressing an antisense RNA against HIV mRNAs under the control of a neuron-specific regulatory sequence.

Antisense nucleic acids expressed under the control of the regulatory sequences of the present invention can be used to treat disorders of a cell type that expresses, or preferably overexpresses, the particular mRNA to which the antisense nucleic acid is directed. In a specific embodiment, such a disorder is an overexpression of a neurotransmitter. In a preferred embodiment, a single-stranded DNA antisense TCAP oligonucleotide is used.

Cell types which express or overexpress a particular mRNA can be identified by various methods known in the art. Such methods include but are not limited to hybridization with a nucleic acid to the gene of interest (e.g. by northern hybridization, dot blot hybridization, in situ hybridization), observing the ability of RNA from the cell type to be translated in vitro into the specific protein produced by the gene, immunoassay, etc. In a preferred aspect, primary tissue from a patient can be assayed for protein expression prior to treatment, e.g., by immunocytochemistry or in situ hybridization.

The amount of antisense nucleic acid that will be effective in the treatment of a particular disorder or condition will depend on the nature of the disorder or condition, and can be determined by standard clinical techniques. Where possible, it is desirable to determine the antisense cytotoxicity to the cell type to be treated in vitro, and then in useful animal model systems prior to testing and use in humans.

In a specific embodiment, pharmaceutical compositions comprising antisense nucleic acids are administered via liposomes, microparticles, or microcapsules. In various embodiments of the invention, it may be useful to use such compositions to achieve sustained release of the antisense nucleic acids. In a specific embodiment, it may be desirable to utilize liposomes targeted via antibodies to specific identifiable tumor antigens (Leonetti et al., Proc. Natl. Acad. Sci. U.S.A. 87: 2448-2451 (1990); Renneisen et al., J. Biol. Chem. 265: 16337-16342 (1990)).

5.7.2. Diagnostic Uses of Nucleic Acid Regulatory Sequences

The nucleotide sequences described herein may also be used as diagnostic tools, where a particular condition or disease state is correlated with polymorphisms among individuals in the CNI-01054, CNI-01056, CNI-01058 or CNI-01059 regulatory sequence. Sequence polymorphisms are the DNA sequence variations that occur between different individuals at the same genetic loci. Polymorphisms can be single nucleotide polymorphisms (SNPs), as well as larger-scale sequence deletions, insertions, or inversions that vary between individuals. Sequence polymorphisms that occur within regulatory DNA sequence can alter the relative levels of gene expression, which in turn can result in a disease condition, susceptibility to a disease, or alter the response of an individual to drug prophylaxis, drug therapy, or other medical treatments. Thus, identifying regulatory sequences and the sequence polymorphisms that occur within them can be used to diagnose a disease or condition, predict the likelihood of developing a disease condition or susceptibility to a condition, predict the likelihood of transmitting an inheritable susceptibility to offspring, or predict the responses of individuals to drug prophylaxis, drug therapies, or other medical treatments.

Methods for detecting SNPs are well known in the art, and generally rely on differential hybridization, i.e., the ability to distinguish between a nucleic acid with full complementarity to a regulatory sequence and a nucleic acid with a single mismatch. The methods can either involve a simple determination of hybridization or lack thereof, or can involve a determination of failure of PCR to produce a product, where the mismatched primer is designed to be mismatched at the more critical 3′ end of the primer. Conventional techniques for detecting SNPs include, e.g., conventional dot blot analysis, single stranded conformational polymorphism (SSCP) analysis (see, e.g., Orita et al., Proc. Natl. Acad. Sci. USA 86:2766-2770 (1989)), denaturing gradient gel electrophoresis (DGGE), heteroduplex analysis, mismatch cleavage detection, and other routine techniques well known in the art (see, e.g., Sheffield et al., Proc. Natl. Acad. Sci. U.S.A. 86:5855-5892 (1989); Grompe, Nature Genetics 5:111-117 (1993)). Other methods are known in the art, for example, solid phase arrays using primer-guided nucleotide incorporation procedures (e.g., Kornher, et al., Nucl. Acids Res. 17:7779-7784 (1989); Sokolov, Nucl. Acids Res. 18:3671 (1990); Syvanen, et al., Genomics 8:684-692 (1990); Kuppuswamy, et al., Proc. Natl. Acad. Sci. U.S.A. 88:1143-1147 (1991); Prezant, et al., Hum. Mutat. 1:159-164 (1992); Ugozzoli, et al., GATA 9:107-112 (1992); Nyren, et al., Anal. Biochem. 208:171-175 (1993); and Wallace WO89/10414). Other methods well known in the art may be used to identify single nucleotide polymorphisms (SNPs), including biallelic SNPs or biallelic markers which have two alleles, both of which are present at a fairly high frequency in a population. Alternative, preferred methods of detecting and mapping SNPs involve microsequencing techniques wherein an SNP site in a target DNA is detecting by a single nucleotide primer extension reaction (see, e.g., Goelet et al., U.S. Pat. No. 6,004,744; Mundy, U.S. Pat. No. 4,656,127; Vary and Diamond, U.S. Pat. No. 4,851,331; Cohen et al., PCT Publication No. WO91/02087; Chee et al., PCT Publication No. WO95/11995; Landegren et al., Science 241:1077-1080 (1988); Nicerson et al., Proc. Natl. Acad. Sci. U.S.A. 87:8923-8927 (1990); Pastinen et al., Genome Res. 7:606-614 (1997); Pastinen et al., Clin. Chem. 42:1391-1397 (1996); Jalanko et al., Clin. Chem. 38:39-43 (1992); Shumaker et al., Hum. Mutation 7:346-354 (1996); Caskey et al., PCT Publication No. WO 95/00669).

5.8. Other Uses

The present invention further provides methods for the use of the nucleic acid regulatory sequences of the invention. In one embodiment, DNA fragments that are found to promote or enhance gene expression may be used to find genes not previously known to be expressed in the nervous system; such genes may include previously unknown genes. The method comprises sequencing the fragment in question, followed by a deduction of the gene or gene-like sequences that the fragment appears to regulate by comparison of the sequence to known genomic sequences using the search algorithms described above. In another embodiment, one can determine the gene associated with a particular regulatory sequence based on sequence homology with a cognate regulatory sequence in another organism, wherein the cognate regulatory sequence in another organism possesses a sequence substantially similar to that of the human regulatory sequence. Such a degree of conservation has been demonstrated for the GAP-43 promoter, known to be found in organisms as evolutionarily diverse as mammals and fish (Reinhard et al., Devel. 120:7167-1775 (1994)).

The regulatory sequence, or fragments thereof, as provided by the present invention may also be used to discover new transcription factors. Though thousands of transcription factors are predicted to exist in humans (see Venter et al., Science 291:1304-1350 (2001)), only a few hundred have been discovered; far fewer have been described as regulating gene expression in the nervous system. Transcription factors binding to the regulatory sequences provided herein may be discovered by any means known to those in the art. For example, fragments of the regulatory sequence can be separated on a non-denaturing agarose or polyacrylamide gel, under conditions allowing for binding of transcription factors to appropriate DNA recognition sequences or elements, in the presence or absence of extracts of cells derived from the nervous system; a shift in the mobility of a particular fragment in the presence of cell extracts indicates that the fragment is being bound by a protein that may regulate transcription. Alternatively, a column can be constructed, comprising a packing material having a fragment of the regulatory sequence available for binding to cell extract components passed through the column, followed by washing of the column with a buffer that allows for DNA-protein interactions; proteins binding to the fragment, including potential new transcription factors, can thereupon be eluted and characterized.

The nucleic acid regulatory sequences of the invention can also be used to aid in the construction of microarrays that allow the simultaneous assessment of the binding of specific transcription factors to a plurality of regulatory DNA sequences. Such a microarray has been reported in the yeast genetic system (Ren et al., Science 290:2306-2309 (2001)), and the techniques utilized therein can be readily utilized in the construction of such micro-arrays. Using the regulatory sequences provided herein, in addition to known regulatory sequences, one can construct a similar microarray for human regulatory DNA sequences in order to profile transcription factor utilization in different cell, tissues, or between different physiological conditions or disease states.

### 6. EXAMPLES

6.1. Identification and Analysis of the Regulatory Sequences

6.1.1. DNA Preparation

Human chromosome 22 DNA libraries were prepared by cloning fragments of BamHI- or PstI-digested human chromosome 22 DNA sequences into the unique BamHI or PstI sites present in the multiple cloning site (MCS) of a plasmid vector constructed at Cogent Neuroscience, Inc (pCOGENT1) (FIG. 1). This plasmid contains a multiple cloning site (MCS) containing unique restriction enzyme sites for BamHI, EcoRI and PstI. Downstream of the MCS, the vector also contains a basal promoter sequence containing a “TATA” box and a reporter gene. The vector also contains an ampicillin resistance gene, and a pMB1-derived origin of DNA replication. A positive control plasmid, pCOGENT1(E), was created by inserting an approximately 400 nucleotide DNA fragment containing the strong transcription enhancer from the CMV immediate early (IE) gene promoter (Boshart et al. Cell 41(2):521-30 (1985)) into the unique EcoRI site in the MCS of pCOGENT1. The vector pCOGENT1, with no library insert, was used as the negative control.

Library transformants were plated and grown on LB agar (DIFCO Laboratories) bioassay plates with 0.2 mg/ml ampicillin at 37° C. for 24 hours. Single colonies were then used to inoculate deep-well blocks containing 1.5 ml LB broth containing 0.2 mg/ml ampicillin. Inoculated cultures were incubated at 37° C. with agitation at 150-200 rpm for 18-24 hours. Replicate plates were created from the cultures by adding 20 μl of culture to 80 μl of LB broth containing 18% glycerol and 0.2 mg/ml ampicillin and stored at −80° C. The remaining bacterial cells inoculated into 15-150 ml of fresh LB broth containing 0.2 mg/ml ampicillin. Following incubation at 37° C. with agitation at 150-200 rpm for 18-24 hours, plasmid DNA was extracted using Promega DNA extraction kits. Purified plasmid DNA was introduced into mammalian nervous system cells.

6.1.2. Evaluation of Modulatory Activity of Cloned Sequences

Purified DNA was introduced into mammalian (rat) brain slice cells in culture. Individual clones were chosen for the presence of human DNA sequences (“regulatory sequences”) that caused detectable expression of the reporter gene under conditions that did not result in detectable expression of the reporter gene when the vector alone was similarly introduced into cells. Positive controls for each genomic clone included a strong positive CMV promoter inserted into the MCS. The negative control was the expression plasmid with no insert. Genomic clones were evaluated for their ability to drive detectable levels of gene expression in nervous system-derived cells, and when active, for cell-type or nervous system-region specificity (or lack thereof).

6.1.3. DNA Sequencing

The nucleotide sequence of a DNA insert that was selected for its ability to cause detectable expression of the reporter gene when introduced into cells was determined using the ABI Big Dye terminator Cycle Sequencing Ready Reaction Kit followed by subsequent analysis on the ABI3700 capillary sequencing machine (PE Biosystems, Foster City, Calif.). Plasmid DNA was annealed with oligonucleotide primers complementary to regions upstream (forward primer) and downstream (reverse primer) of the MCS. Cycle sequencing reactions were carried out in a thermocycler (PCR machine) using standard methods. The extension products from the sequencing reaction were purified by precipitation using isopropanol and analyzed on the ABI3700 sequencer according to the manufacturer's protocol.

6.1.4. Sequence Analysis

The sequence data for the nucleic acid regulatory sequences was compared using the BLAST 2.0 algorithm (Altschul et al., Nucleic Acids Res. 25:3389 (1997)) against known sequences in the GenBank sequence database maintained by NCBI (National Center for Biotechnology Information). This program uses the two-hit method to find homology within the database. The BLAST nucleotide searches were performed with the “BLAST N” program (wordlength=11).

Predictions of transcription factor binding sites were made using GeneTools software from BioTools, Inc. (BTI). The eukaryotic transcription factors and DNA motifs from the Transcription Factor Database (TFD) are located on the Internet, via file transfer protocol, at ncbi.nlm.nih.gov/repository/TFD. Information present in the University of California, Santa Cruz (UCSC), draft assembly of the human genome (available on the Internet at genome.ucsc.edu/goldenPath/octTracks.html) was used to position the regulatory sequence on human chromosome 22.

6.2. Nucleic Acid Regulatory Sequence CNI-01054

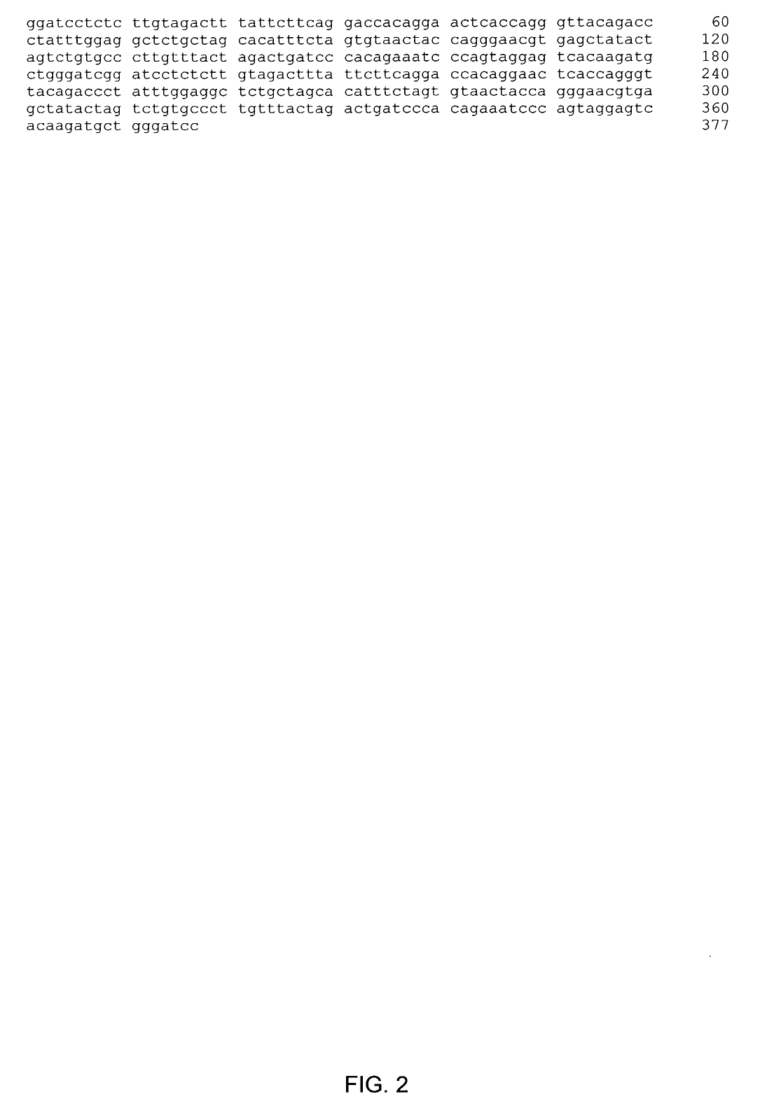

The sequence of the 377 nucleotide CNI-01054 regulatory sequence is shown in FIG. 2. A BLAST analysis showed homology to the sequences disclosed in GenBank accession numbers AP000555 and AE001301. GenBank accession number AP000555 is Homo sapiens genomic DNA from chromosome 22q11.2. GenBank accession number AE001301 is Chlamydia trachomatis section 28 of 87 of the complete genome.

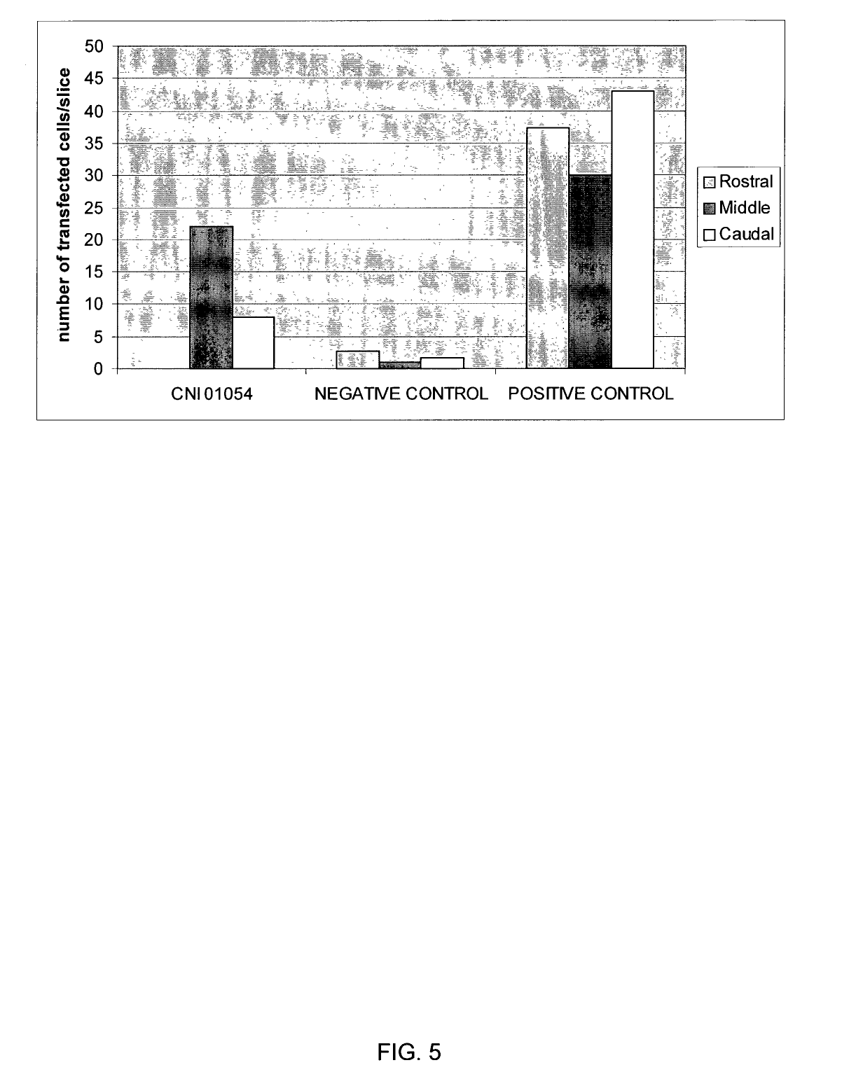

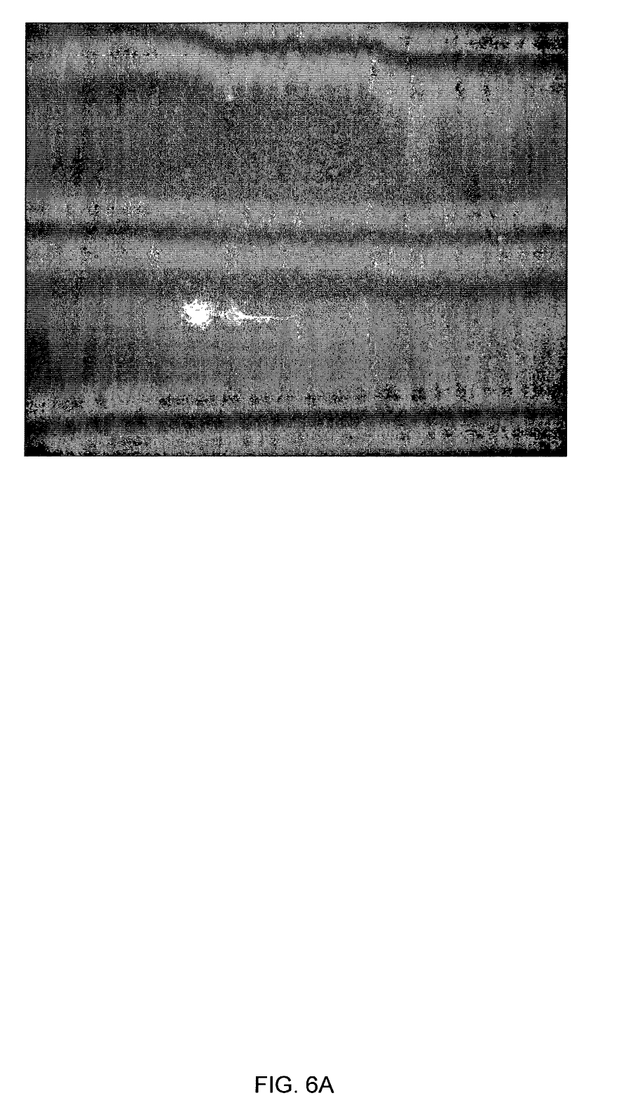

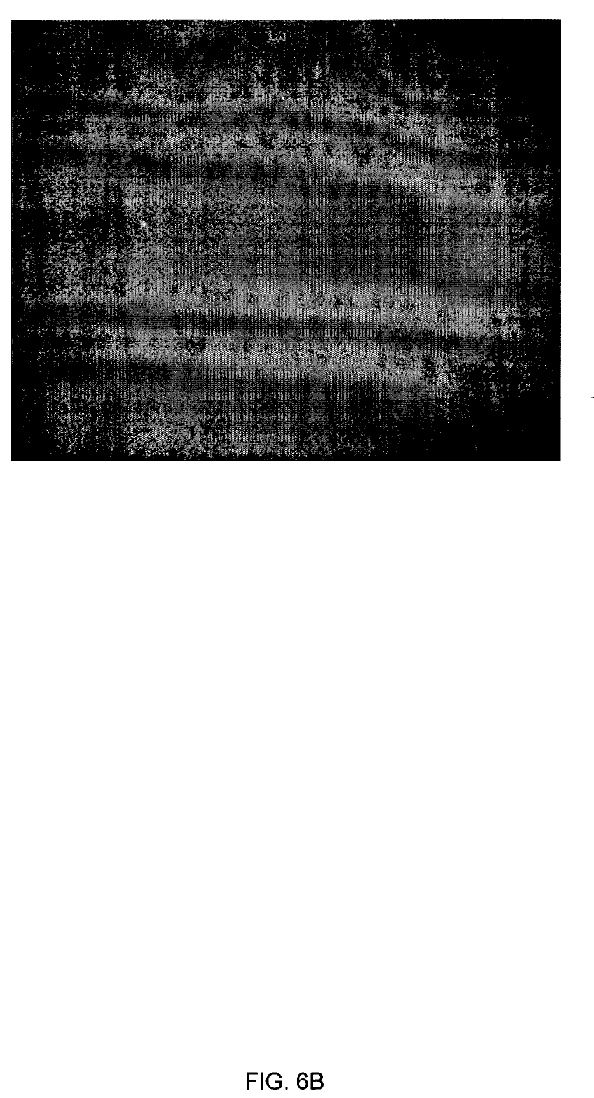

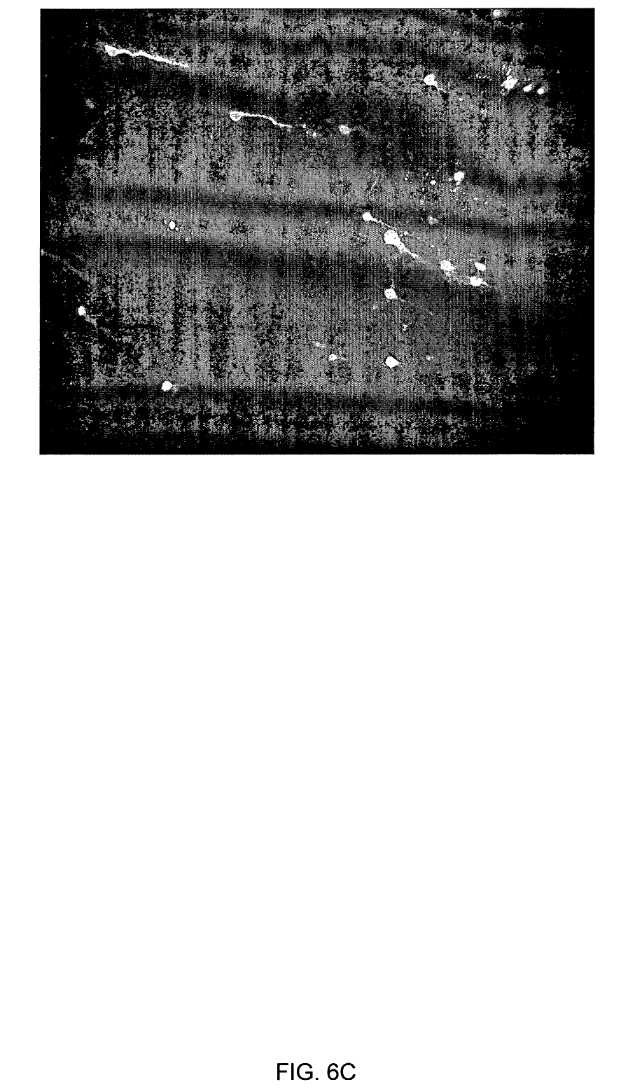

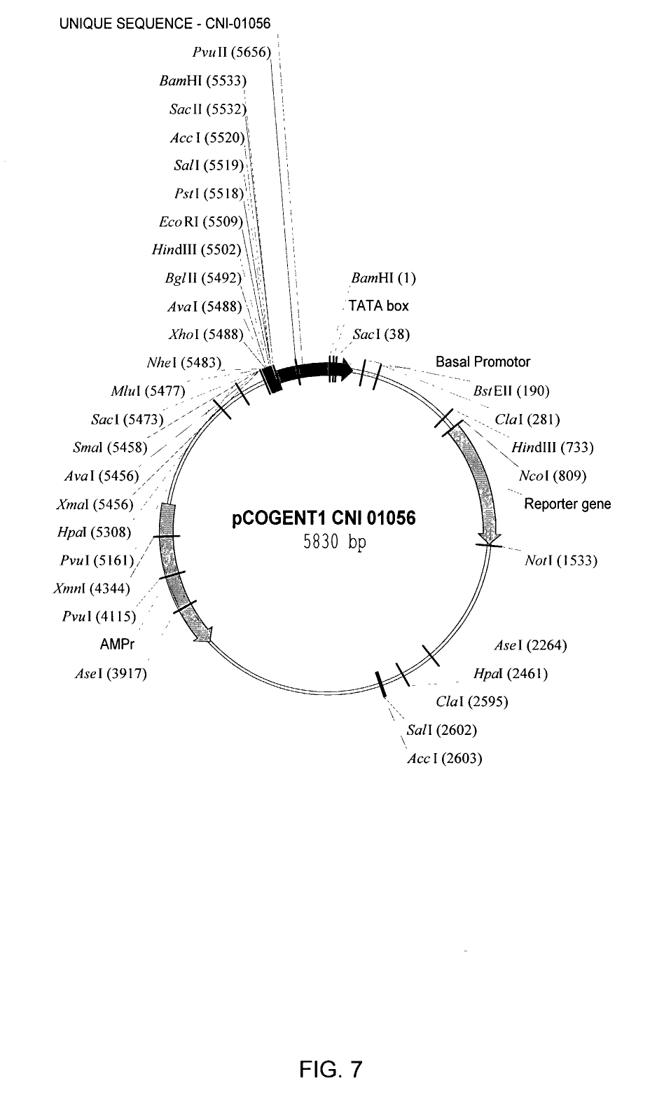

As depicted in FIG. 3 (UCSC linkage map of a region of human chromosome 22), the sequence of CNI-01054 is located within an intron of the gene encoding mitogen-activated kinase 1 (MAP kinase 1; also known as ERK, ERK2, p41mapk, p38, p38, MAPK2, PRKM2). The sequence of the predicted gene is the complement of base positions 18870527 to 18766943. The sequence of CNI-01054 is in the same orientation as the predicted gene. The 5′ end of the predicted gene is approximately 67287 base pairs “upstream” of the 5′ end of the sequence of CNI-01054. The CNI-01054 nucleotide sequence was analyzed for transcription factor recognition sites (FIG. 4.) Genomic clone CNI-01054 caused expression of the reporter gene above the level for the negative control pCOGENT1 (FIG. 5). Expression in the middle region of the brain was greater than in caudal and rostral regions. Nervous system cells transfected with CNI-01054 clearly show expression in brain slices (FIGS. 6A-6C).

6.3. Nucleic Acid Regulatory Sequence CNI-01056

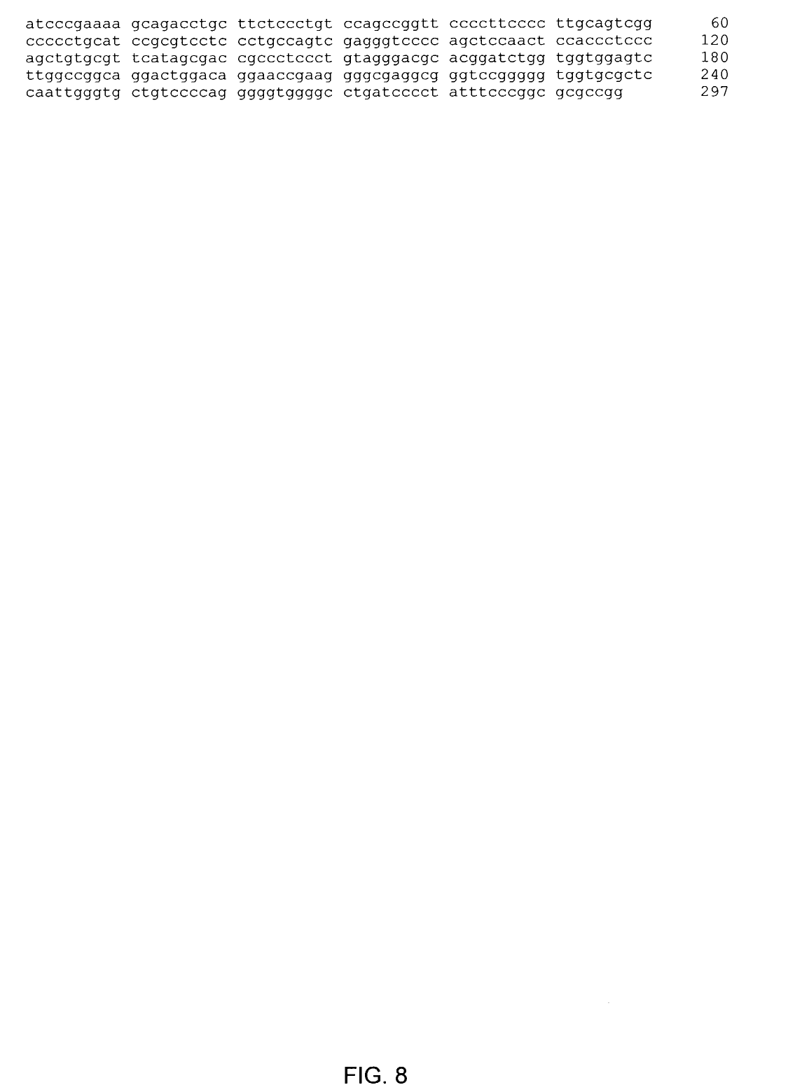

The sequence of the 297 nucleotide CNI-01056 regulatory sequence is shown in FIG. 8. A BLAST analysis showed the highest homology to GenBank accession number AF240786, a clone encoding Homo sapiens glutathione S-transferase theta 2 (GSTT2) and glutathione S-transferase theta 1 (GSTT1) coding regions (397/304), and GenBank accession number Z84718, which is chromosome 22q 1-12 clone 322B1 (297/304).

As depicted in FIG. 9 (UCSC linkage map of a region of human chromosome 22), the sequence of CNI-01056 is present in two locations in the sequence of human chromosome 22, each within approximately 20,000 bp of the other. In both locations, the sequence of CNI-01056 is positioned between two genes, one encoding D-dopachrome tautomerase (DTT) and the other encoding glutathione S-transferase theta 2 (GSTT2). In both locations, the two genes are present in opposite but non-overlapping orientations. The genes are arranged “head-to-head”, in that the 5′ ends of the genes are close to each other, which would result in transcription of the two genes in opposite directions. In both locations, the 5′ ends of the two genes are in such close proximity that they are essentially joined by the 297 nucleotide sequence of CNI-01056. Thus, it is possible that the sequence of CNI-01056 regulates the expression of both genes at both locations on human chromosome 22. If the (+)-sense DNA strand of chromosome 22 is defined as the strand whose sequence is oriented 5′ to 3′ from centrome to telomere on the long arm of chromosome 22, and the complementary strand is the (−)-sense strand, then the two sets of genes relative to the sequence of CNI-01056 are arranged as follows from centromere to telomere. The first set is arranged GSTT2, (−)-sense (20945658 to 20949367); CNI-01056, (−)-sense (20949437 to 20949741); DTT, (+)-sense (20949737 to 20,955,799). The second set is arranged DTT, (−)-sense (20,959,606 to 20,968,068); CNI-01056, (+)-sense (20968063 to 20968367); GSTT2, (+)-sense (20968373 to 20972147). The CNI-01056 nucleotide sequence was analyzed for transcription factor recognition sites (FIG. 10).

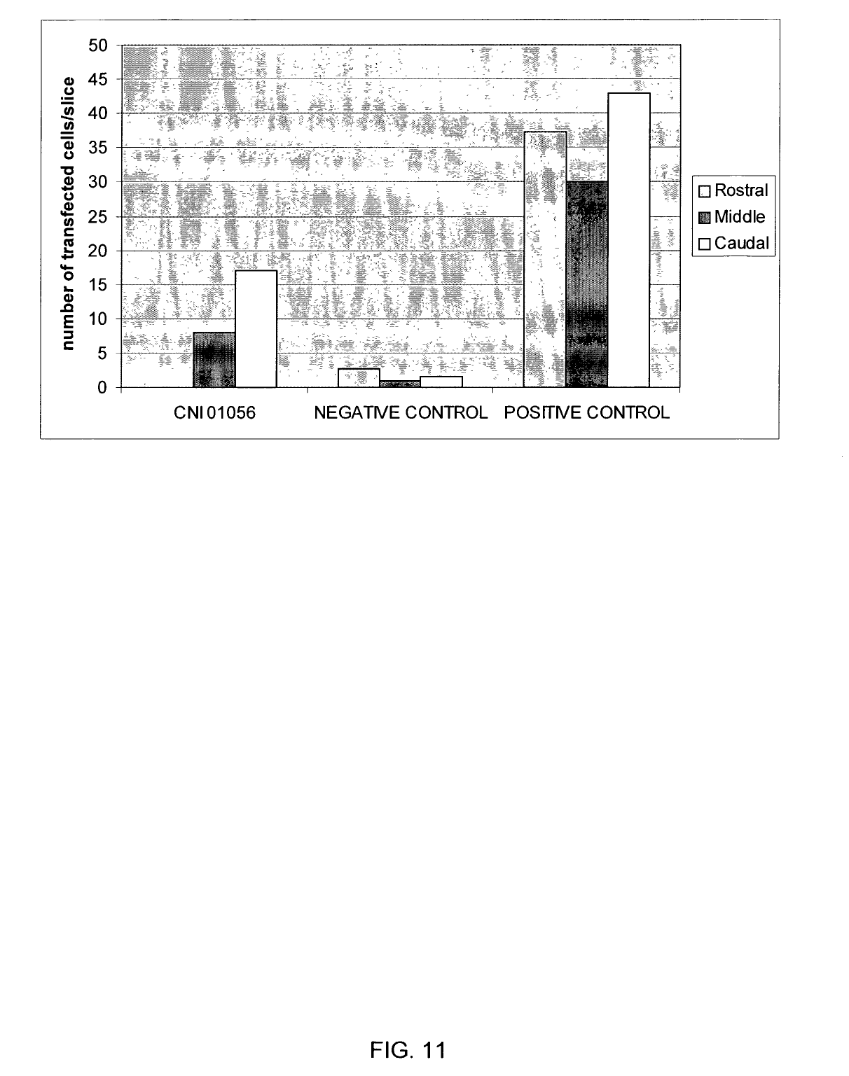

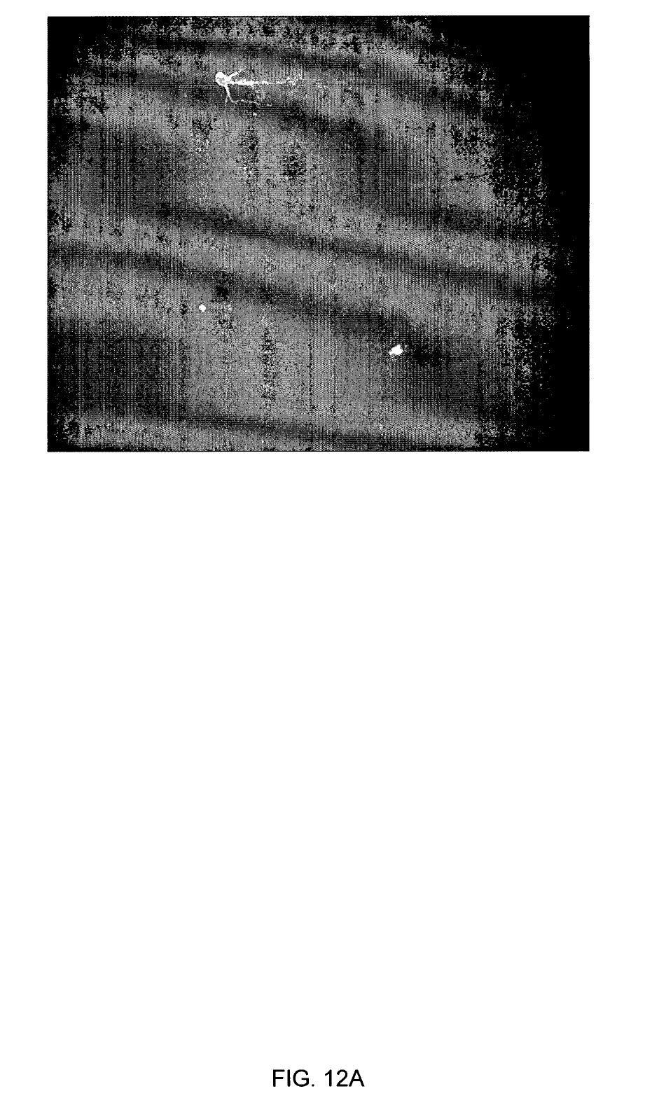

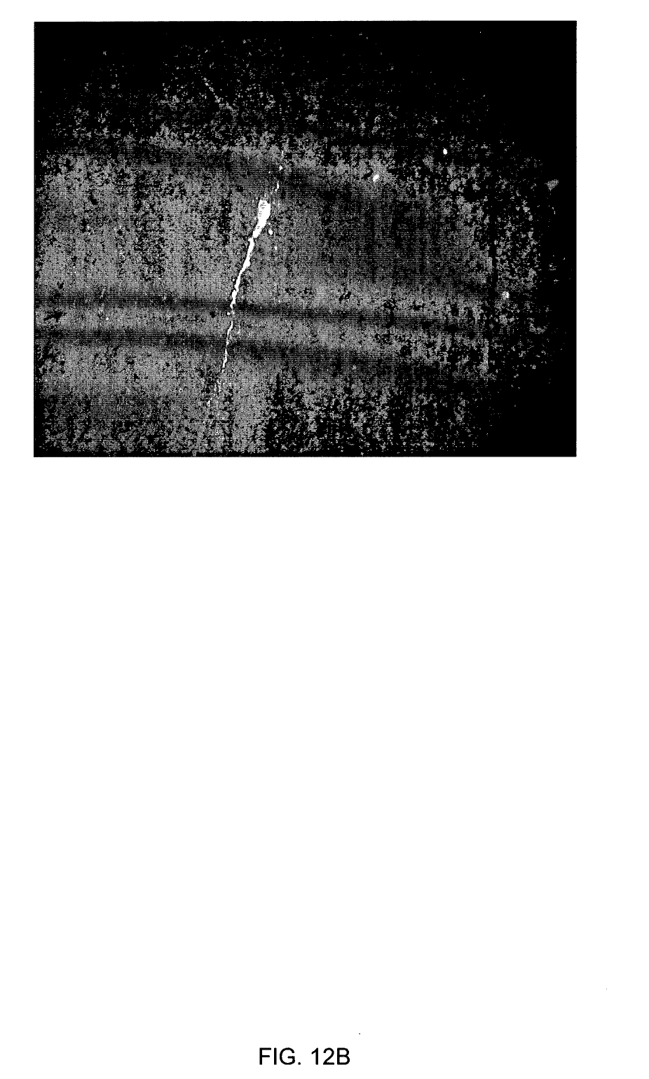

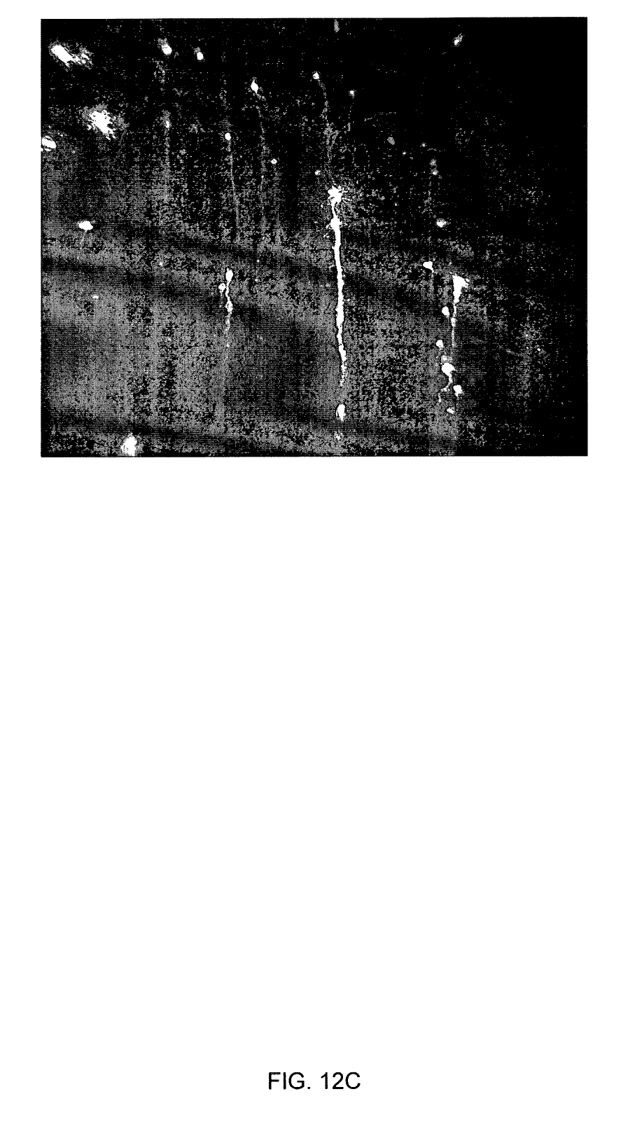

Genomic clone CNI-01056 caused expression of the reporter gene above the level for the negative control pCOGENT1 (FIG. 11). The expression of the reporter gene is higher in the caudal region than that of the middle region, and lower in the rostral region. Nervous system cells transfected with CNI-01056 clearly show expression in brain slices (FIG. 12).

6.4. Nucleic Acid Regulatory Sequence CNI-01058

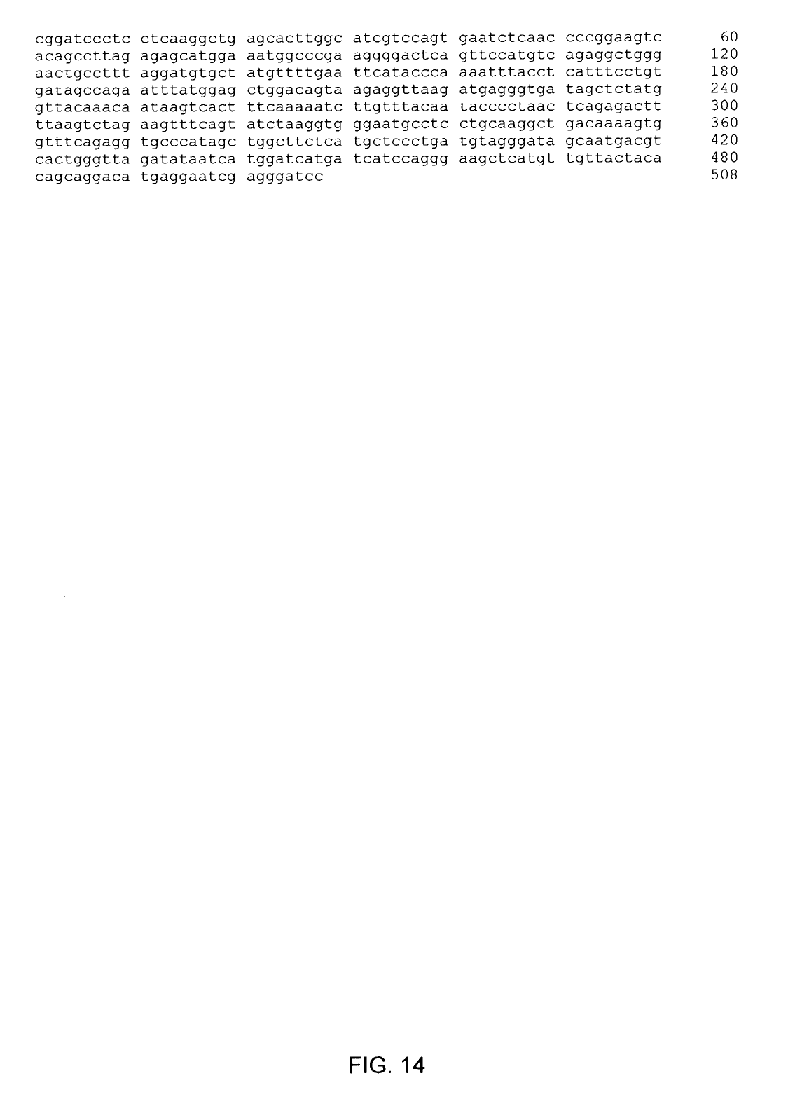

The sequence of the 508 nucleotide CNI-01058 regulatory sequence is shown in FIG. 14. A BLAST analysis showed the highest homology to GenBank accession number Z95114, which is clone CTA-212A2 encoding Human DNA sequence on chromosome 22q12, and GenBank accession number AC016021, which is bacterial artificial chromosome clone (BAC) BACR27F05.

As depicted in FIG. 15 (UCSC linkage map of a region of human chromosome 22), the nearest known or predicted gene to the sequence of CNI-01058 is a predicted gene, C22000498. The sequence of the predicted gene is the complement of the sequence at positions 33144492 to 33148852. The sequence of CNI-01058 is in the opposite orientation as the predicted gene and is located “upstream” of the predicted gene. The 3′ end of the predicted gene is approximately 4957 base pairs “downstream” of the 3′ end of the sequence of CNI-01058. The gene is predicted to encode human apolipoprotein L, 3 (TNF-inducible protein CG12-1) (GenBank Accession No.: NP_{—}055164; REFSEQ accession NM_{—}014349.1 Horrevoets, “Vascular endothelial genes that are responsive to tumor necrosis factor-alpha in vitro are expressed in atherosclerotic lesions, including inhibitor of apoptosis protein-1, stannin, and two novel genes,” Blood 93, 3418-3431 (1999)). The CNI-01058 nucleotide was analyzed for transcription factor recognition sites (FIG. 16).

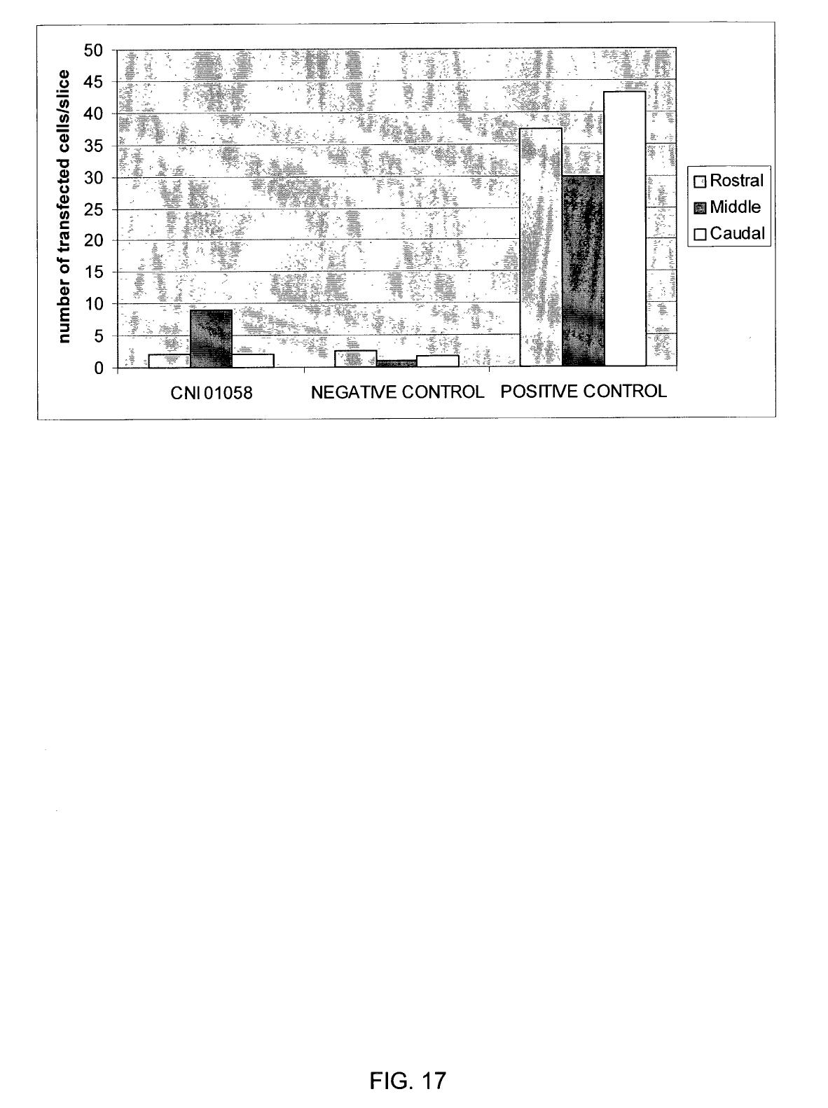

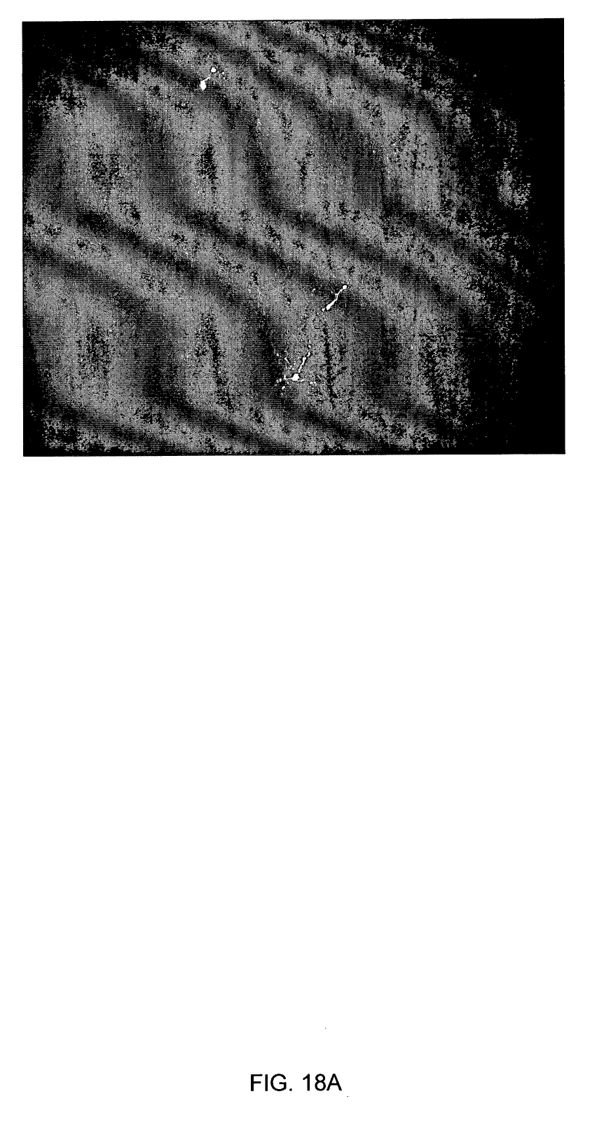

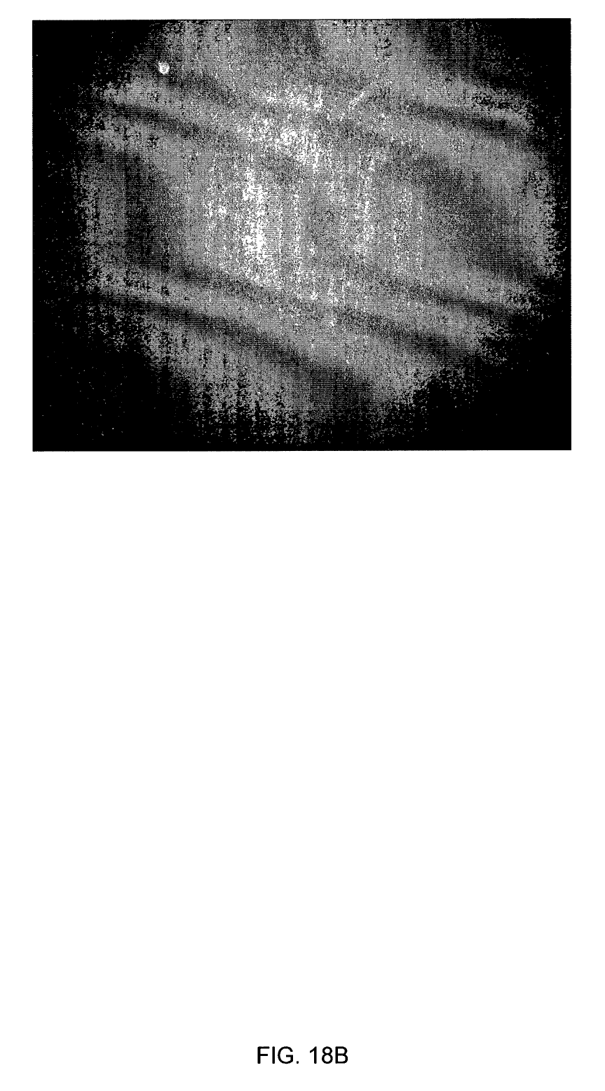

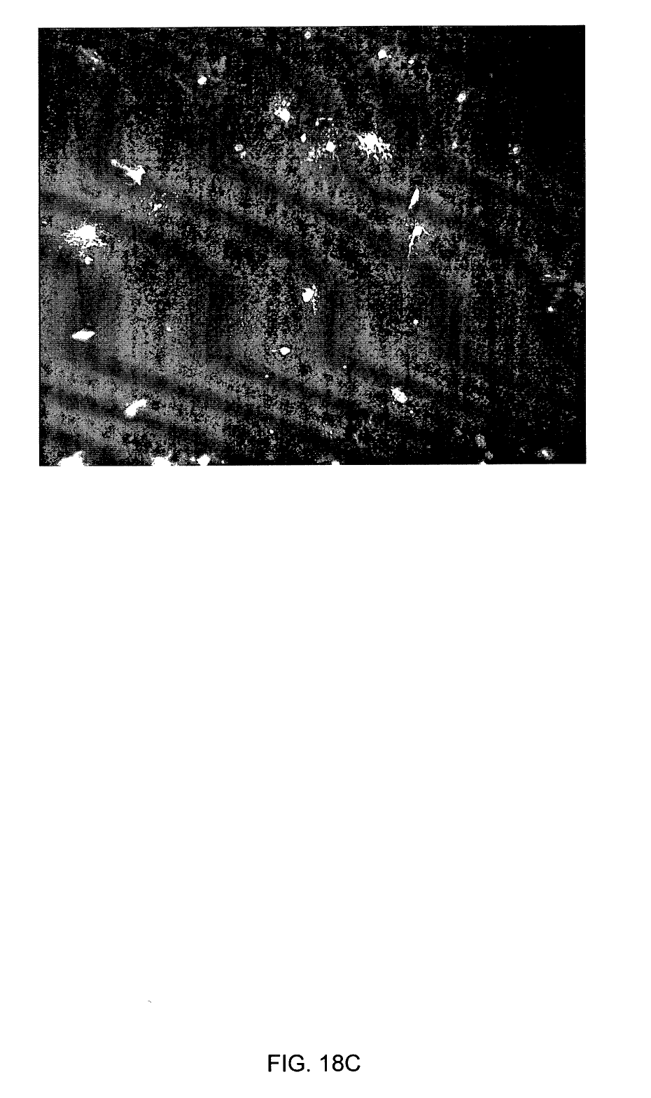

Genomic clone CNI-01058 caused expression of the reporter gene above the level for the negative control pCOGENT1 (FIG. 17). The expression of the reporter gene is highest in the middle region of the brain, with lower expression in the rostral and caudal regions. Nervous system cells transfected with CNI-01058 clearly show expression in brain slices (FIG. 18).

6.5. Nucleic Acid Regulatory Sequence CNI-01059

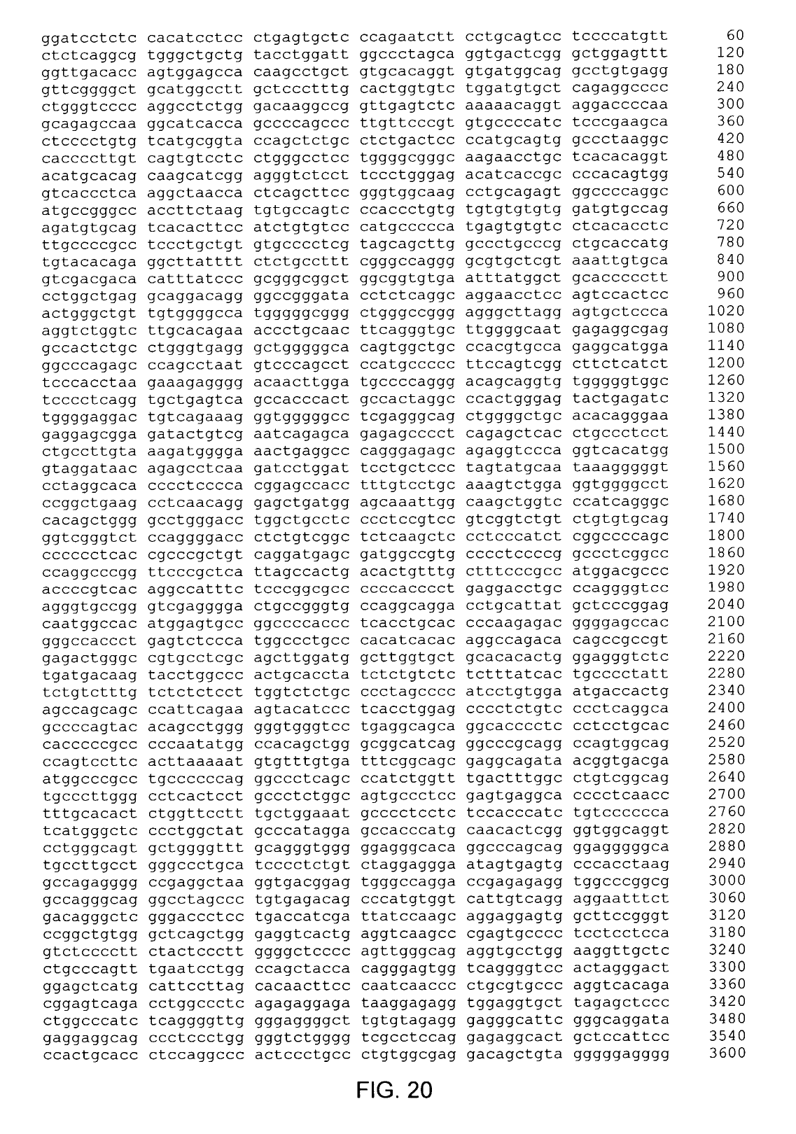

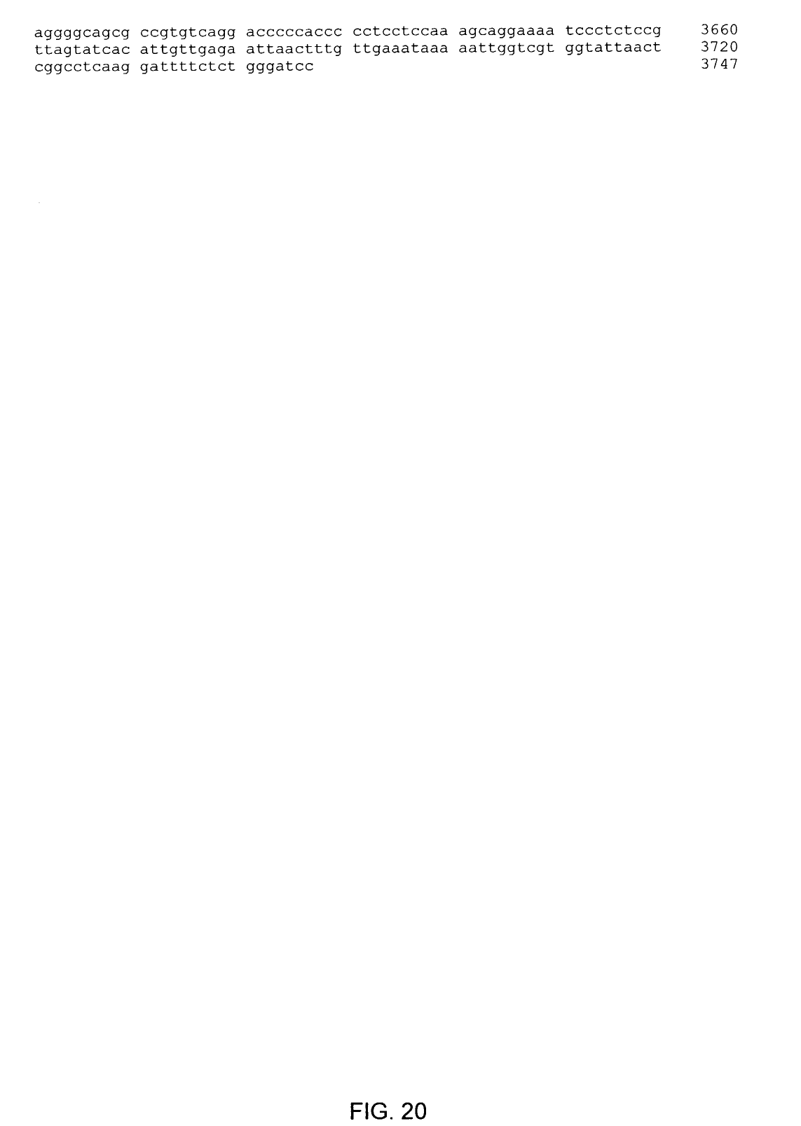

The sequence of the 3747 nucleotide CNI-01059 regulatory sequence is shown in FIG. 20. A BLAST analysis showed the highest homology to GenBank accession number AC000093, which is Homo sapiens Chromosome 22Q11 Cosmid Clone carlaa, and GenBank accession number AC008780, which is Homo sapiens chromosome 5 clone CTD-2023N9.

As depicted in FIG. 21 (UCSC linkage map of a region of human chromosome 22), the sequences of three known genes are located near the sequence of CNI-01059. The three genes are peanut (Drosophila)-like 1 (PNUTL1; aliases HCDCREL-1 and H5) located at positions 16642156 to 16650973; glycoprotein Ib (platelet), beta polypeptide (GP1BB) located at positions 16651197 to 16652425; and T-box 1 (TBX1), located at positions 16684357 to 16711247. The sequences of the three genes and CNI-01059 are in the same orientation, arrange in order, 5′ to 3′: PNUTL1, GP1BB, CNI-01059, TBX1. The 3′ ends of the PNUTL1 and GP 1 BB genes are respectively approximately 11734 and 10282 nucleotides from the 5′ end of the sequence of CNI-01059. The 3′ end of the sequence of CNI-01059 is approximately 17903 nucleotides from the 5′ end of the TBX1 gene. The CNI-01059 nucleotide sequence was analyzed for transcription factor recognition sites (FIG. 22).

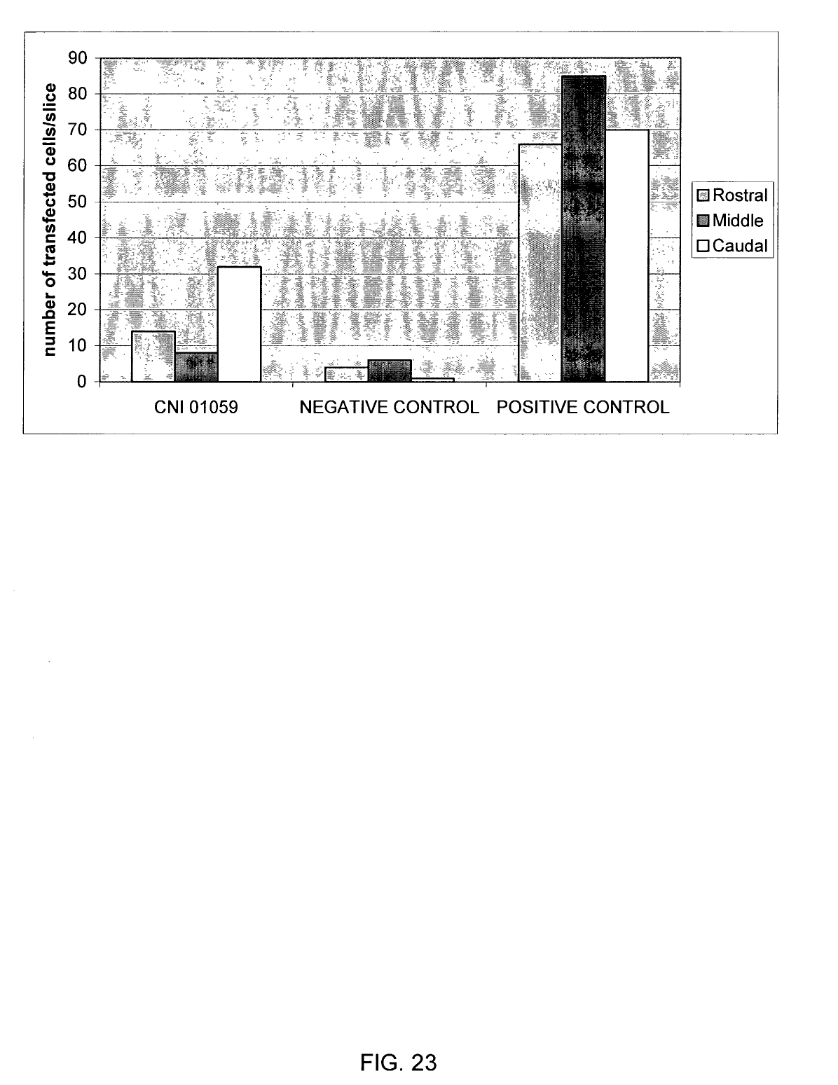

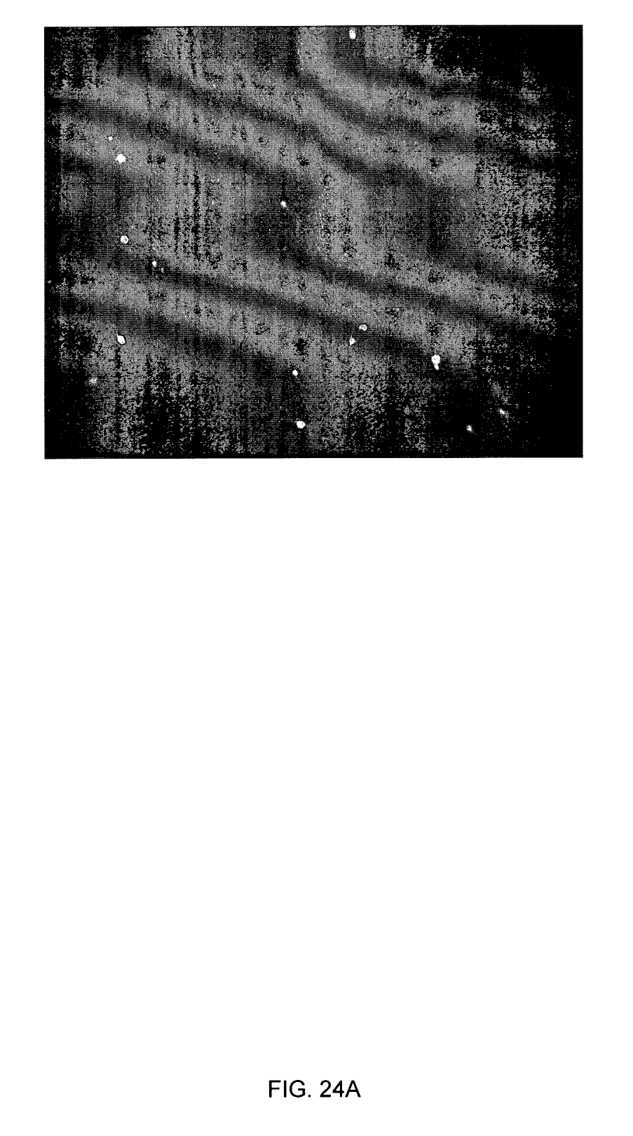

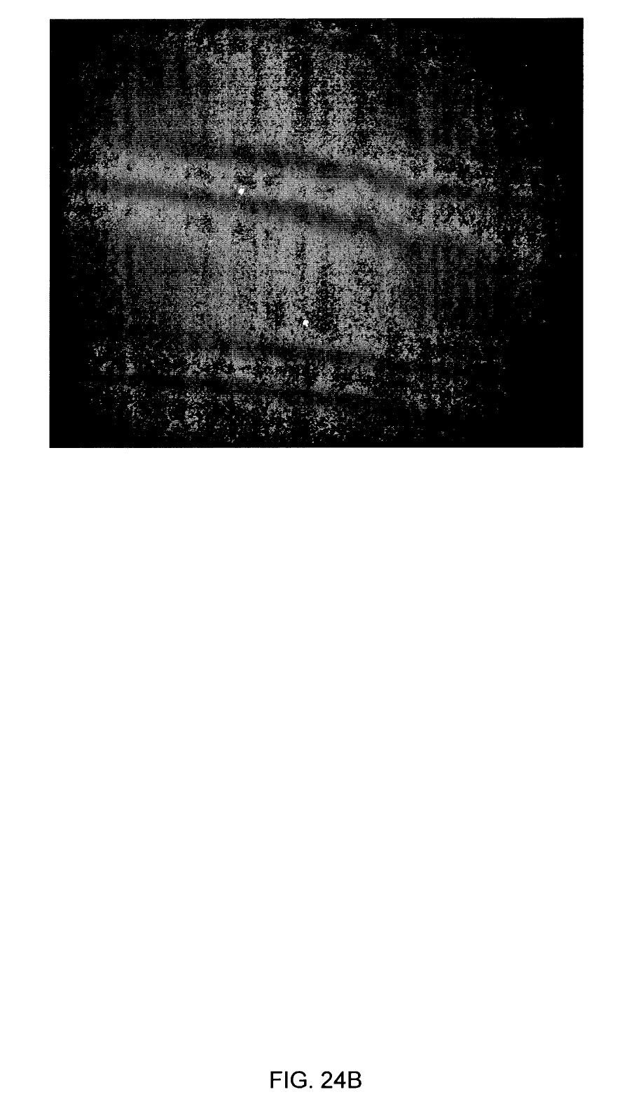

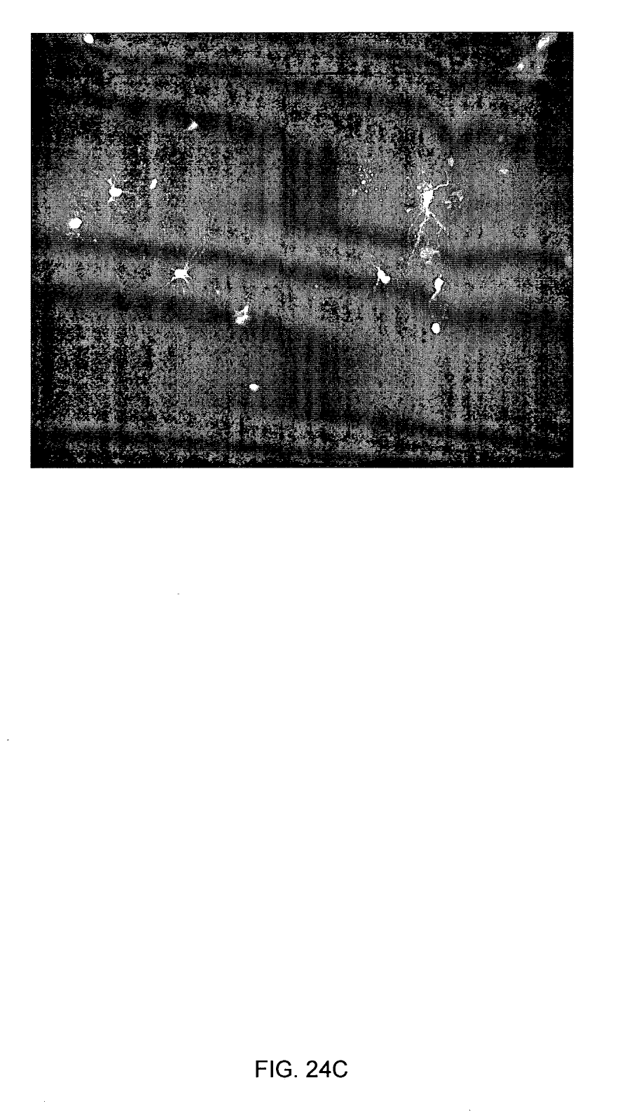

Genomic clone CNI-01059 caused expression of the reporter gene above the level for the negative control pCOGENT1 (FIG. 23.) The expression of the reporter gene is higher in the caudal region of the brain than in the rostral and middle regions. Nervous system cells transfected with CNI-01059 clearly show expression in brain slices (FIG. 24).

What is claimed is:

1. An isolated nucleic acid molecule comprising the nucleotide sequence of SEQ ID NO: 1, SEQ ID NO: 2, SEQ ID NO: 3, or SEQ ID NO: 4.

2. An isolated nucleic acid regulatory sequence molecule comprising a transcription activating nucleotide sequence of SEQ ID NO: 1, SEQ ID NO: 2, SEQ ID NO: 3, or SEQ ID NO: 4.

3. The isolated nucleic acid regulatory sequence molecule of claim 2, wherein the isolated nucleic acid regulatory sequence molecule is a restriction fragment of SEQ ID NO: 1, SEQ ID NO: 2, SEQ ID NO: 3, or SEQ ID NO: 4.

4. The isolated nucleic acid regulatory sequence molecule of claim 2, wherein the isolated nucleic acid regulatory sequence is created by nuclease digestion of a nucleic acid molecule comprising SEQ ID NO: 1, SEQ ID NO: 2, SEQ ID NO: 3, or SEQ ID NO: 4.

5. The isolated nucleic acid molecule of claim 1, wherein the isolated nucleic acid molecule is operably linked to a nucleic acid molecule comprising a coding sequence.

6. The isolated nucleic acid regulatory sequence molecule of any one of claims 2-4, wherein the isolated nucleic acid regulatory sequence molecule is operably linked to a nucleic acid molecule comprising a coding sequence.

7. An isolated nucleic acid molecule comprising the reverse complement of the nucleotide sequence of SEQ ID NO: 1, SEQ ID NO: 2, SEQ ID NO: 3, or SEQ ID NO: 4.

8. An isolated nucleic acid regulatory sequence molecule comprising the reverse complement of the nucleotide sequence of the nucleic acid regulatory sequence molecule of claim 2.

9. The isolated nucleic acid regulatory sequence molecule of claim 2, wherein the transcription activating nucleotide sequence comprises at least about 50 contiguous nucleotides of SEQ ID NO: 1, SEQ ID NO: 2, SEQ ID NO: 3, or SEQ ID NO: 4.

10. An isolated nucleic acid regulatory sequence molecule comprising a transcription activating nucleotide sequence that hybridizes over its entire length to the nucleotide sequence of SEQ ID NO: 1, SEQ ID NO: 2, SEQ ID NO: 3, or SEQ ID NO: 4 or the complement thereof.

11. A vector comprising the nucleotide sequence of claim 1 or claim 2.

12. The vector of claim 11 further comprising a coding sequence operably linked to the nucleotide sequence.

13. The vector of claim 12, wherein the coding sequence is heterologous to the nucleotide sequence.

14. The vector of claim 11 further comprising a multiple cloning site (MCS), wherein when a coding sequence is inserted into the MCS, the coding sequence is operably linked to the nucleotide sequence.

15. The vector of claim 11, further comprising an internal ribosomal entry site (IRES).

16. The vector of claim 12, wherein the coding sequence is heterologous to the nucleotide sequence.

17. The vector of any one of claims 12 or 14, wherein said coding sequence is a reporter gene sequence.

18. The vector of any one of claims 12 or 14, wherein said coding sequence is a neuroprotective sequence.

19. The vector of claim 17, wherein said reporter gene sequence encodes β-galactosidase, a fluorescent protein, chloramphenicol acetyltransferase, luciferase or an antigenic marker.

20. A vector comprising a promoter and an MCS operably linked in an upstream-to-downstream order, and the nucleotide sequence of claim 1 or claim 2 or a transcription activating nucleotide sequence thereof.

21. The vector of claim 20, further comprising an internal ribosomal entry site (IRES).

22. The vector of claim 20, wherein when a coding sequence is present within the MCS, the coding sequence is operably linked to said promoter sequence and to the nucleotide sequence or transcription activating nucleotide sequence thereof.

23. The vector of claim 20, wherein said promoter is heterologous to the coding sequence.

24 The vector of claim 20, wherein the vector is adapted for transfer to a eukaryotic host cell.

25. The vector of claim 24, wherein the eukaryotic host cell is a nervous system cell.

26. The vector of claim 25, wherein the nervous system cell is a nervous system cell line, glial cell, astrocyte, oligodendrocyte, mesencephalic neuron, hypothalamic neuron or cortical neuron.

27. The vector claim 20, wherein said vector is adapted for transfer to a prokaryotic host cell.

28. A host cell, or progeny thereof, comprising the vector of claim 11.

29. The host cell of claim 28, wherein said host cell is a eukaryotic cell.

30. The host cell of claim 29, wherein said host cell is a nervous system cell.

31. The host cell of claim 28, wherein said host cell is a prokaryotic cell.

32. A kit comprising the vector of claim 11, 25, or 28.

33. A kit comprising the host cell of claim 29.

34. A kit comprising the host cell of claim 31.

35. A transgenic non-human animal comprising the nucleotide sequence of claim 1 or claim 2, wherein the nucleotide sequence is heterologous to said nonhuman animal.

36. The transgenic animal of claim 35, wherein said nucleotide sequence is contained within an episome.

37. The transgenic animal of claim 35, wherein said nucleotide sequence is inserted into the genome of said animal by homologous recombination.

38. The transgenic animal of claim 35, wherein said nucleotide sequence is inserted into the genome of said animal by nonhomologous recombination.

39. The transgenic animal of claim 37 or 38 wherein said nucleotide sequence promotes or enhances expression of a coding sequence in the genome of said animal.

40. A method of expressing a coding sequence in a host cell in cell culture, comprising culturing a host cell of claim 28 under conditions effective to allow expression of the coding sequence by said host cell.

41. The method of claim 40, wherein said host cell is a nervous system cell.

42. The method of claim 40, wherein said vector exists within said host cell as an episome.

43. The method of claim 40, wherein said vector is present in the genome of said host cell.

44. The method of claim 43, wherein said vector is introduced into the genome of said host cell by homologous recombination.

45. The method of claim 43, wherein said vector is introduced into the genome of said host cell by nonhomologous recombination.

46. The method of claim 43, wherein said nucleic acid sequence controls expression of a coding sequence endogenously present in the genome of said host cell.

47. A method of producing a polypeptide comprising:
(a) introducing the vector of claim 11 into a host cell such that a nucleotide sequence contained within said vector promotes or enhances the expression of a coding sequence; and
(b) maintaining said host cell under conditions effective to allow expression of said coding sequence, and to allow translation of mRNA,
wherein said expression of said coding sequence produces a polypeptide.

48. The method of claim 47, wherein the vector is present in the genome of said host cell.

49. The method of claim 48, wherein said vector is introduced into the genome of said host cell by homologous recombination.

50. The method of claim 48, wherein said vector is introduced into the genome of said host cell by nonhomologous recombination.

51. A method of identifying a modulator of a regulatory sequence active in nervous system-derived host cells comprising:
(a) contacting the nervous system-derived host cell containing the vector of claim 9 with a test compound; and
(b) detecting a change of expression of the reporter gene, relative to its expression in the absence of the test compound, such that, if a change is detected, a modulator of the nucleic acid regulatory sequence is identified.

52. The method of claim 51, wherein said regulatory sequence active in nervous system-derived cells is SEQ ID NO: 1, SEQ ID NO: 2, SEQ ID NO: 3, or SEQ ID NO: 4 or a transcription activating sequence thereof.

53. The method of claim 51, wherein said reporter gene encodes β-galactosidase, a fluorescent protein, chloramphenicol acetyltransferase, luciferase or an antigenic marker.

54. A method of constructing a transgenic animal comprising introducing the nucleic acid molecule of claim 1 or claim 2 into an embryonic host cell.
